(19) United States
(12) Patent Application Publication
(10) Pub. No.: US 2011/0184530 A1
(43) Pub. Date: Jul. 28, 2011

(54) High performance reticulated elastomeric matrix preparation, properties, reinforcement, and use in surgical devices, tissue augmentation and/or tissue repair

(75) Inventors: Arindam Datta, Hillsborough, NJ (US); Lawrence P. Lavelle, JR., Rahway, NJ (US); Craig Friedman, Westport, CT (US); John D. MacGillivray, Greenwich, CT (US); Aisa Sendijarevic, Troy, MI (US)

(73) Assignee: Biomerix Corporation, New York, NY (US)

(21) Appl. No.: 12/868,521

(22) Filed: Aug. 25, 2010

Related U.S. Application Data
(63) Continuation of application No. 11/652,763, filed on Jan. 11, 2007, now abandoned.
Application No. 11/652,763 is a continuation-in-part of application No. 10/848,624, filed on May 17, 2004, now Pat. No. 7,803,395.
(60) Provisional application No. 60/816,120, filed on Jun. 22, 2006.
(60) Provisional application No. 60/849,328, filed on Oct. 3, 2006.

(51) Int. Cl. A61F 2/02

(52) U.S. Cl. 623/23.72

(57) ABSTRACT

This invention relates to reticulated elastomeric matrices, their manufacture, their post-processing, such as their reinforcement, compressive molding or annealing, and uses including uses for implantable devices into or for topical treatment of patients, such as humans and other animals, for surgical devices, tissue augmentation, tissue repair, therapeutic, nutritional, or other useful purposes.

This application is a continuation of U.S. application Ser. No. 11/652,763, filed Jan. 11, 2007, which is a continuation-in part of U.S. application Ser. No. 10/848,624, filed May 17, 2004, and claims the benefit of that application and of U.S. provisional application No. 60/816,120, filed Jun. 22, 2006, and U.S. provisional application No. 60/849,328, filed Oct. 3, 2006, the disclosure of each application being incorporated by reference herein in its entirety.

### FIELD OF THE INVENTION

This invention relates to reticulated elastomeric matrices, their manufacture, including by so-called “hand” techniques and “machine” methods, their post-processing, such as their reinforcement, compressive molding or annealing, and uses including uses for implantable devices into or for topical treatment of patients, such as humans and other animals, for surgical devices, tissue augmentation, tissue repair, therapeutic, nutritional, or other useful purposes. For these and other purposes the inventive products may be used alone or may be loaded with one or more deliverable substances.

### BACKGROUND OF THE INVENTION

The tissue engineering (“TE”) approach generally includes the delivery of a biocompatible tissue substrate that serves as a scaffold or support onto which cells may attach, grow and/or proliferate, thereby synthesizing new tissue by regeneration or new tissue growth to repair a wound or defect. Open cell biocompatible foams have been recognized to have significant potential for use in the repair and regeneration of tissue. However, because of their ability to break down and be absorbed by the body without causing any adverse tissue response during and after the body has synthesized new tissue to repair the wound, prior work in this area has focused on tissue engineering scaffolds made from synthetic bioabsorbable materials.

The major weaknesses of these approaches relating to bioabsorbable three-dimensional porous scaffolds used for tissue regeneration are undesirable tissue response during the product's life cycle as the polymers biodegrade and the inability to engineer the degradation characteristics of the TE scaffold in vivo, thus severely limiting their ability to serve as effective scaffolds. Also, there remains a need for an implant that withstands compression in a delivery-device during delivery to a biological site, e.g., by a catheter, endoscope, arthoscope or syringe, capable of expansion by resiliently recovering to occupy and remain in the biological site, and of a particular pore size such that the implant can become ingrown with tissue at that site to serve a useful therapeutic purpose. Furthermore, many materials produced from polyurethane foams formed by blowing during the polymerization process are unattractive from the point of view of biodurability because undesirable materials that can produce adverse biological reactions are generated during polymerization, for example, carcinogens, cytotoxins and the like. In contrast, the biodurable reticulated elastomeric matrix materials of the present invention are suitable for such applications as long-term TE implants, especially where dynamic loadings and/or extensions are experienced, such as in soft tissue related orthopedic applications.

Most current tissue scaffolds are made from biodegradable polymers such as homopolymers and copolymers of polyglycolic acid (“PGA”), polylactic acid (“PLA”), and the like or biopolymers such as collagen, elastin, animal tissue-based products, human tissue-based products and the like. These materials suffer from many disadvantages, for example, it is difficult to engineer their properties to approximate those of various targeted tissues. Additionally, their capacity to retain their performance in vivo is short lived, especially when it pertains to their elastomeric and resilient properties. For tissues that take several weeks or months to regenerate, remodel and/or heal, such as orthopedic soft tissues or vascular tissues, scaffolds made from biodegradable polymers and biopolymers cannot be used because they cannot maintain the underlying performance demanded of an effective scaffold and, particularly for biolpolymers, degrade in approximately 2 to 4 weeks. Some biodegradable polymers may survive up to one year or more in vivo but they are usually brittle, having a tensile elongation to break of less than about 5% under in vivo or in vitro environments. Most tissue engineering matrices of scaffolds made from biopolymers and in some cases for biodegradable polymers usually have a high probability of undesired tissue response and device rejection. The latter is especially true for animal or human tissue-based products. Undesirable tissue response is often observed for biodegradable polymeric implants when they break down and degrade during the long-term healing of chronic tissue defects.

Alternatively, lyophilization techniques and leachable porogens such as salt and sugar are currently used make porous scaffolds from biodegradable polymers; however, control over the properties, porosities and structure of the resulting scaffolds is poor.

The implantable devices of this invention comprising a reticulated elastomeric matrix overcome the above-described problems of bioabsorbable materials, biodegradable polymers and biopolymers. These reticulated elastomeric matrix materials can be engineered to substantially match the properties of the tissue that is being targeted for repair or to meet the particular requirements of a specific application that will lead to regeneration, remodeling or healing of tissues. Ways to successfully engineer their properties to approximate those of various targeted tissues or properties so that regeneration, remodeling and/or healing of tissues are promoted are disclosed herein.

Disclosed herein are methods to engineer the morphology and/or properties of the reticulated elastomeric matrices of the present invention by controlling their chemistry, processing and post-processing features, such as the amount of cross-linking, amount of crystallinity, chemical composition, curing conditions, degree of reticulation and/or post-reticulation processing, such as annealing, compressive molding and/or incorporating reinforcement. Unlike biodegradable polymers, a reticulated elastomeric matrix maintains its physical characteristics and performance in vivo over long periods of time. Thus, it does not initiate undesirable tissue response as is observed for biodegradable implants when they break down and degrade.

Unlike biodegradable polymers or biopolymers, an implantable device of this invention comprising reticulated elastomeric matrix can maintain its physical characteristics and performance in vivo over long periods of time. It does not initiate undesirable tissue response as is observed for biodegradable implants when they break down and degrade. The high void content and degree of reticulation of the reticulated elastomeric matrix of this invention allows tissue ingrowth and proliferation of cells within the matrix. Without being bound by any particular theory, it is believed that the high void content and degree of reticulation of the reticulated elastomeric matrix not only allows for tissue ingrowth and proliferation of cells within the matrix but also allows for orientation and remodeling of the healed tissue after the initial tissues have grown into the implantable device. The reticulated elastomeric matrix and/or the implantable device, over time, provides functionality, such as load bearing capability, of the original tissue that is being repaired or replaced. Without being bound by any particular theory, it is believed that owing to the high void content of the reticulated elastomeric matrix or implantable device comprising it, once the tissue is healed and bio-integration takes place, most of the regenerated or repaired site consists of new tissue and a small volume fraction of the reticulated elastomeric matrix, or the implantable device formed from it.

Also, the capacity for compression set, resilience and/or dynamic compression recovery of the implantable device is engineered to provide a high recovery force of the reticulated elastomeric matrix after repetitive cyclic loading. Such a feature is particularly advantageous in uses, e.g., in orthopedic uses, in which cyclic loading of the implantable device might otherwise permanently compress the reticulated elastomeric matrix, thereby preventing it from achieving the substantially continuous contact with the surrounding soft tissues necessary to promote optimal cellular infiltration and tissue ingrowth. In another non-limiting example, the density and pore size of an implantable device of the present invention is engineered to maximize permeability of the reticulated elastomeric matrix under compression. Such features are advantageous if high loads are placed on the implantable device. In yet another non-limiting example, the properties of the reticulated elastomeric matrix are engineered to maximize its “soft, conformal fit,” which is particularly advantageous in cosmetic surgical applications.

U.S. Pat. Nos. 5,891,558 to Bell et al., 6,306,424 to Vyakarnam et al., 6,638,312 to Plouhar et al., and 6,599,323 to Melican et al. and United States Patent Application Publication Nos. US 2002/0131989 to Brown et al., US 2003/0147935 and US 2004/0078077 each to Binette et al., and US 2004/0175408 to Chun et al. each describe a composite implant or scaffold.

The reference “Innovative Manufacture of Olefin Foams” by A. E. S. Clarke et al., Paper 17 in the proceedings of Blowing Agents and Foaming Processes 2006, May 16-17, 2006 (Munich, Germany) describes the preparation of olefin foams by conventional heating to expand the surface of the material and microwave heating to expand the interior.

The foregoing description of background art may include insights, discoveries, understandings or disclosures, or associations together of disclosures, that were not known to the relevant art prior to the present invention but which were provided by the invention. Some such contributions of the invention may have been specifically pointed out herein, whereas other such contributions of the invention will be apparent from their context. Merely because a document may have been cited here, no admission is made that the field of the document, which may be quite different from that of the invention, is analogous to the field or fields of the invention. The citation of any reference in the background section of this application is not an admission that the reference is prior art to the application.

### SUMMARY OF THE INVENTION

The implantable devices of the invention are useful for many applications as long-term TE implants, especially where dynamic loadings and/or extensions are experienced, such as in soft tissue related orthopedic applications for repair and regeneration.

The present invention is directed to an implantable device comprising a reticulated resiliently-compressible elastomeric matrix comprising a plurality of pores, where the implantable device further comprises a reinforcement in at least one dimension. The implantable device can be annealed before or after being reinforced. The implantable device can be compressive molded before or after being reinforced.

The present invention is also directed to an implantable device comprising a reticulated resiliently-compressible elastomeric matrix comprising a plurality of pores, where the implantable device is compressive molded after it is reticulated. The implantable device can be annealed before or after being compressive molded. The implantable device can be reinforced before or after being compressive molded.

The present invention is also directed to an implantable device comprising a reticulated resiliently-compressible elastomeric matrix comprising a plurality of pores, where the implantable device is annealed after it is reticulated. The implantable device can be reinforced before or after being annealed. The implantable device can be compressive molded before or after being annealed.

The present invention is also directed to a polymerization process for preparing an elastomeric matrix, the process having the steps of admixing:
a) 100 parts by weight of a polyol component,
b) from about 10 to about 90 parts by weight of an isocyanate component,
c) from about 0.5 to about 6.0 parts by weight of a blowing agent,
d) optionally, from about 0.05 to about 8.0 parts by weight of a cross-linking agent,
e) optionally, from about 0.05 to about 8.0 parts by weight of a chain extender,
f) optionally, from about 0.05 to about 3.0 parts by weight of at least one catalyst,
g) optionally, from about 0.1 to about 8.0 parts by weight of at least one cell opener,
h) from about 0.1 to about 8.0 parts by weight of a surfactant, and
i) optionally, up to about 15 parts by weight of a viscosity modifier;
   to provide the elastomeric matrix.

The present invention is also directed to a process for preparing an at least partially reticulated elastomeric matrix, the process having the steps of
1) admixing:
   a) 100 parts by weight of an elastomeric material,
   b) optionally, from about 2 to about 70 parts by weight of a more hydrophilic polymeric material,
   c) optionally, from about 0.1 to about 20 parts by weight of a cross-linking agent, and
   d) optionally, from about 1 to about 20 parts by weight of a blowing agent to form a mixture;
2) exposing the mixture to microwave irradiation at a frequency of from about 2.2 GHz to about 6.0 GHz, optionally while also heating the mixture to a temperature of from about 70° C. to about 225° C.;
   to provide the at least partially reticulated elastomeric matrix.

The present invention is also directed to an implantable device containing a reticulated elastomeric matrix, where the reticulated elastomeric matrix is configured to permit cellular ingrowth and proliferation into the annealed reticulated elastomeric matrix.

The present invention is also directed to a method of treating a tissue defect, the method having the steps of:
a) optionally compressing the implantable device of the invention from a relaxed configuration to a first, compact configuration;
b) delivering the compressed implantable device to the in vivo site of the defect via a delivery-device; and
c) optionally allowing the implantable device to expand to a second, working configuration at the in vivo site.

The present invention is also directed to a method of treating a tissue defect, the method having the step of inserting the implantable device of the invention by an open surgical procedure.

The tissue defect can relate to an orthopedic application, general surgical application, cosmetic surgical application, tissue engineering application, or any mixture thereof. The orthopedic application can relate to a repair, reconstruction, regeneration, augmentation, gap interposition, or any mixture thereof of a tendon, ligament, cartilige, meniscus, spinal disc, or any mixture thereof. The general surgical application can relate to an inguinal hernea, a ventral abdominal hernea, a femoral hernea, an umbilical hernea, or any mixture thereof.

The present invention is also directed to the at least partially reticulated elastomeric matrix product of any of the methods described herein for making it.

### BRIEF DESCRIPTION OF THE DRAWINGS

Some embodiments of the invention, and of making and using the invention, are described in detail below, which description is to be read with and in the light of the foregoing description, by way of example, with reference to the accompanying drawings, in which like reference characters designate the same or similar elements throughout the several views, and in which:

FIG. 1 is a schematic view showing one possible morphology for a portion of the microstructure of one embodiment of a porous biodurable elastomeric product according to the invention;

FIG. 2 is a schematic block flow diagram of a process for preparing a porous biodurable elastomeric implantable device according to the invention;

FIG. 3 illustrates an exemplary compressive molding process for a cylindrical preform;

FIG. 4 illustrates an exemplary compressive molding process for a cubical preform;

FIG. 5 illustrates several different exemplary reticulated elastomeric matrix reinforcement grids;

FIG. 6 illustrates several different exemplary reticulated elastomeric matrix reinforcement grids;

FIG. 7 illustrates the geometry of the suture pullout strength test;

FIG. 8 illustrates regions amenable to cosmetic facial surgery for minimally invasive and other reconstructive applications using the implantable device of the present invention;

FIG. 9 illustrates two methods for anchoring a reinforced implantable device to a tuberosity;

FIG. 10 is a scanning electron micrograph image of Reticulated Elastomeric Matrix 1 of Example 5;

FIG. 11 is a plot the Darcy permeability vs. available flow area for several reticulated elastomeric matrices;

FIG. 12 is a scanning electron micrograph image of Reticulated Elastomeric Matrix 3 of Example 7;

FIG. 13 shows the pattern of the rectangular implantable device of Example 14;

FIG. 14 shows the dimensions for features of the pattern of the rectangular implantable device of Example 14; and

FIG. 15 shows a histology analysis photograph of the device of Example 15.

### DETAILED DESCRIPTION OF THE INVENTION

Certain embodiments of the invention comprise reticulated biodurable elastomer products, which are also compressible and exhibit resilience in their recovery, that have a diversity of applications and can be employed, by way of example, in biological implantation, especially into humans, for long-term TE implants, especially where dynamic loadings and/or extensions are experienced, such as in soft tissue related orthopedic applications; for tissue augmentation, support and repair; for therapeutic purposes; for cosmetic, reconstructive, urologic or gastroesophageal purposes; or as substrates for pharmaceutically-active agent, e.g., drug, delivery. Other embodiments involve reticulated biodurable elastomer products for in vivo delivery via catheter, endoscope, arthoscope, laproscop, cystoscope, syringe or other suitable delivery-device and can be satisfactorily implanted or otherwise exposed to living tissue and fluids for extended periods of time, for example, at least 29 days.

There is a need in medicine, as recognized by the present invention, for innocuous implantable devices that can be delivered to an in vivo patient site, for example a site in a human patient, that can occupy that site for extended periods of time without being harmful to the host. In one embodiment, such implantable devices can also eventually become integrated, such as biointegrated, e.g., ingrown with tissue or bio-integrated. Various biodegradable or absorbable porous polymeric materials have been proposed for tissue augmentation and repair.

It would be desirable to form implantable devices suitable for use as tissue engineering scaffolds, or other comparable substrates, to support in vivo cell propagation applications, for example in a large number of orthopedic applications especially in soft tissue attachment, regeneration, augmentation, support and ingrowth of a prosthetic organ. Without being bound by any particular theory, having a high void content and a high degree of reticulation is thought to allow the implantable device to become at least partially ingrown and/or proliferated, in some cases substantially ingrown and proliferated, in some cases completely ingrown and proliferated, with cells including tissues such as fibroblasts, fibrous tissues, synovial cells, bone marrow stromal cells, stem cells and/or fibrocartilage cells. The ingrown and/or proliferated tissues thereby provide functionality, such as load bearing capability, for defect repair of the original tissue that is being repaired or replaced. However, prior to the advent of the present invention, materials and/or products meeting the requirements for such implantable devices have not been available.

Broadly stated, certain embodiments of the reticulated biodurable elastomeric products of the invention comprise, or are largely if not entirely, constituted by a highly permeable, reticulated matrix formed of a biodurable polymeric elastomer that is resiliently-compressible so as to regain its shape after delivery to a biological site. In one embodiment, the elastomeric matrix has good fatigue resistance associated with dynamic loading. In another embodiment, the elastomeric matrix is chemically well-characterized. In another embodiment, the elastomeric matrix is physically well-characterized. In another embodiment, the elastomeric matrix is chemically and physically well-characterized.

Certain embodiments of the invention can support cell growth and permit cellular ingrowth and proliferation in vivo and are useful as in vivo biological implantable devices, for example, for tissue engineering scaffolds that may be used in vitro or in vivo to provide a substrate for cellular propagation.

The implantable devices of the invention are useful for many applications as long-term tissue engineering implants, especially where dynamic loadings and/or extensions are experienced, such as in soft tissue related orthopedic applications for repair and regeneration. In some embodiments, the reticulated elastomeric matrices of the present invention are as described in U.S. patent application Ser. No. 10/848,624, filed May 17, 2004 (published as U.S. Patent Application Publication No. US 2005-0043816-A1 on Feb. 24, 2005), which is hereby incorporated by reference in its entirety for all purposes.

In one embodiment, the reticulated elastomeric matrix of the invention facilitates tissue ingrowth by providing a surface for cellular attachment, migration, proliferation and/or coating (e.g., collagen) deposition. In another embodiment, any type of tissue can grow into an implantable device comprising a reticulated elastomeric matrix of the invention, including, by way of example, epithelial tissue (which includes, e.g., squamous, cuboidal and columnar epithelial tissue), connective tissue (which includes, e.g., areolar tissue, dense regular and irregular tissue, reticular tissue, adipose tissue, cartilage and bone), and muscle tissue (which includes, e.g., skeletal, smooth and cardiac muscle), or any combination thereof, e.g., fibrovascular tissue. In another embodiment of the invention, an implantable device comprising a reticulated elastomeric matrix of the invention can have tissue ingrowth substantially throughout the volume of its interconnected pores.

In one embodiment, the invention comprises an implantable device having sufficient resilient compressibility to be delivered by a “delivery-device”, i.e., a device with a chamber for containing an elastomeric implantable device while it is delivered to the desired site then released at the site, e.g., using a catheter, endoscope, arthoscope, laproscope, cystoscope or syringe. In another embodiment, the thus-delivered elastomeric implantable device substantially regains its shape after delivery to a biological site and has adequate biodurability and biocompatibility characteristics to be suitable for long-term implantation. In another embodiment, the thus-delivered elastomeric implantable device can span defects and serve as to bridge a gap in the native tissue.

The structure, morphology and properties of the elastomeric matrices of this invention can be engineered or tailored over a wide range of performance by varying the starting materials and/or the processing conditions for different functional or therapeutic uses.

Without being bound by any particular theory, it is thought that an aim of the invention, to provide a light-weight, durable structure that can fill a biological volume or cavity and containing sufficient porosity distributed throughout the volume, can be fulfilled by permitting one or more of occlusion, embolization, cellular ingrowth, cellular proliferation, tissue regeneration, cellular attachment, drug delivery, enzymatic action by immobilized enzymes, and other useful processes as described herein including, in particular, the applications to which priority is claimed.

In one embodiment, elastomeric matrices of the invention have sufficient resilience to allow substantial recovery, e.g., to at least about 50% of the size of the relaxed configuration in at least one dimension, after being compressed for implantation in the human body, for example, a low compression set, e.g., at 25° C. or 37° C., and sufficient strength and flow-through for the matrix to be used for controlled release of pharmaceutically-active agents, such as a drug, and for other medical applications. In another embodiment, elastomeric matrices of the invention have sufficient resilience to allow recovery to at least about 60% of the size of the relaxed configuration in at least one dimension after being compressed for implantation in the human body. In another embodiment, elastomeric matrices of the invention have sufficient resilience to allow recovery to at least about 90% of the size of the relaxed configuration in at least one dimension after being compressed for implantation in the human body.

In the present application, the term “biodurable” describes elastomers and other products that are stable for extended periods of time in a biological environment. Such products should not exhibit significant symptoms of breakdown or degradation, erosion or significant deterioration of mechanical properties relevant to their employment when exposed to biological environments for periods of time commensurate with the use of the implantable device. The period of implantation may be weeks, months or years; the lifetime of a host product in which the elastomeric products of the invention are incorporated, such as a graft or prosthetic; or the lifetime of a patient host to the elastomeric product. In one embodiment, the desired period of exposure is to be understood to be at least about 29 days. In another embodiment, the desired period of exposure is to be understood to be at least 29 days. In one embodiment, the implantable device is biodurable for at least 2 months. In another embodiment, the implantable device is biodurable for at least 6 months. In another embodiment, the implantable device is biodurable for at least 12 months. In another embodiment, the implantable device is biodurable for longer than 12 months. In another embodiment, the implantable device is biodurable for at least 24 months. In another embodiment, the implantable device is biodurable for at least 5 years. In another embodiment, the implantable device is biodurable for longer than 5 years.

In one embodiment, biodurable products of the invention are also biocompatible. In the present application, the term “biocompatible” means that the product induces few, if any, adverse biological reactions when implanted in a host patient. Similar considerations applicable to “biodurable” also apply to the property of “biocompatibility”.

An intended biological environment can be understood to in vivo, e.g., that of a patient host into which the product is implanted or to which the product is topically applied, for example, a mammalian host such as a human being or other primate, a pet or sports animal, a livestock or food animal, or a laboratory animal. All such uses are contemplated as being within the scope of the invention. As used herein, a “patient” is an animal. In one embodiment, the animal is a bird, including but not limited to a chicken, turkey, duck, goose or quail, or a mammal. In another embodiment, the animal is a mammal, including but not limited to a cow, horse, sheep, goat, pig, cat, dog, mouse, rat, hamster, rabbit, guinea pig, monkey and a human. In another embodiment, the animal is a primate or a human. In another embodiment, the animal is a human.

In one embodiment, structural materials for the inventive porous elastomers are synthetic polymers, especially but not exclusively, elastomeric polymers that are resistant to biological degradation, for example, in one embodiment, polycarbonate polyurethanes, polycarbonate urea-urethanes, polyether polyurethanes, poly(carbonate-co-ether) urea-urethanes, polysiloxanes and the like, in another embodiment polycarbonate polyurethanes, polycarbonate urea-urethanes, poly(carbonate-co-ether) urea-urethanes and polysiloxanes, in another embodiment polycarbonate polyurethanes, polycarbonate urea-urethanes, and polysiloxanes. Such elastomers are generally hydrophobic but, pursuant to the invention, may be treated to have surfaces that are less hydrophobic or somewhat hydrophilic. In another embodiment, such elastomers may be produced with surfaces that are less hydrophobic or somewhat hydrophilic.

The reticulated biodurable elastomeric products of the invention can be described as having a “macrostructure” and a “microstructure”, which terms are used herein in the general senses described in the following paragraphs.

The “macrostructure” refers to the overall physical characteristics of an article or object formed of the biodurable elastomeric product of the invention, such as: the outer periphery as described by the geometric limits of the article or object, ignoring the pores or voids; the “macrostructural surface area” which references the outermost surface areas as though any pores thereon were filled, ignoring the surface areas within the pores; the “macrostructural volume” or simply the “volume” occupied by the article or object which is the volume bounded by the macrostructural, or simply “macro”, surface area; and the “bulk density” which is the weight per unit volume of the article or object itself as distinct from the density of the structural material.

The “microstructure” refers to the features of the interior structure of the biodurable elastomeric material from which the inventive products are constituted such as pore dimensions; pore surface area, being the total area of the material surfaces in the pores; and the configuration of the struts and intersections that constitute the solid structure of certain embodiments of the inventive elastomeric product.

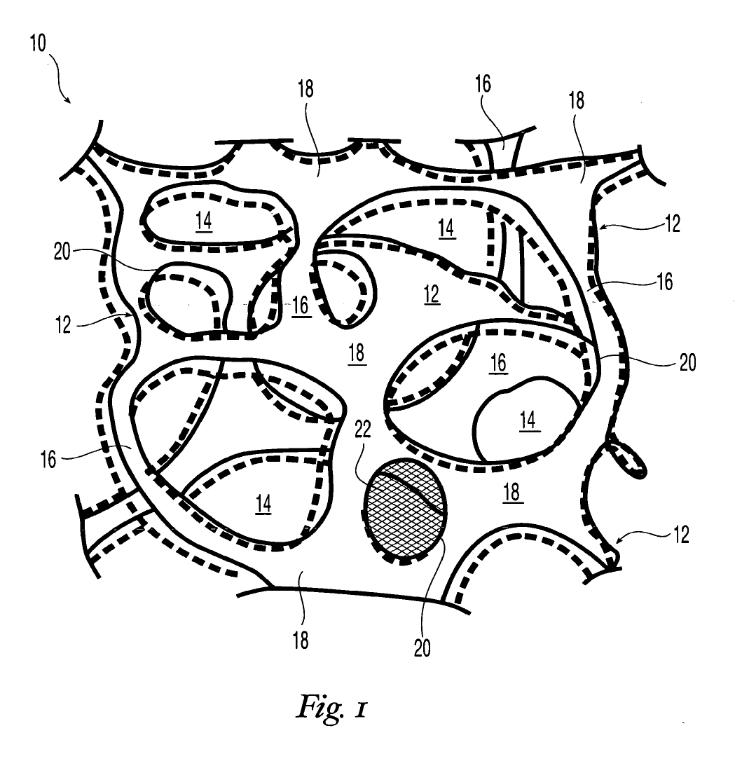

Referring to FIG. 1, what is shown for convenience is a schematic depiction of the particular morphology of a reticulated foam. FIG. 1 is a convenient way of illustrating some of the features and principles of the microstructure of some embodiments of the invention. This figure is not intended to be an idealized depiction of an embodiment of, nor is it a detailed rendering of a particular embodiment of the elastomeric products of the invention. Other features and principles of the microstructure will be apparent from the present specification, or will be apparent from one or more of the inventive processes for manufacturing porous elastomeric products that are described herein.

Morphology

Described generally, the microstructure of the illustrated porous biodurable elastomeric matrix 10, which may, inter alia, be an individual element having a distinct shape or an extended, continuous or amorphous entity, comprises a reticulated solid phase 12 formed of a suitable biodurable elastomeric material and interspersed therewithin, or defined thereby, a continuous interconnected void phase 14, the latter being a principle feature of a reticulated structure.

In one embodiment, the elastomeric material of which elastomeric matrix 10 is constituted may be a mixture or blend of multiple materials. In another embodiment, the elastomeric material is a single synthetic polymeric elastomer such as will be described in more detail below. In other embodiments, although elastomeric matrix 10 is subjected to post-reticulation processing, such as annealing, compressive molding and/or reinforcement, it is to be understood that the elastomeric matrix 10 retains its defining characteristics, that is, it remains biodurable, reticulated and elastomeric.

Void phase 14 will usually be air- or gas-filled prior to use. During use, void phase 14 will in many but not all cases become filled with liquid, for example, with biological fluids or body fluids.

Solid phase 12 of elastomeric matrix 10, as shown in FIG. 1, has an organic structure and comprises a multiplicity of relatively thin struts 16 that extend between and interconnect a number of intersections 18. The intersections 18 are substantial structural locations where three or more struts 16 meet one another. Four or five or more struts 16 may be seen to meet at an intersection 18 or at a location where two intersections 18 can be seen to merge into one another. In one embodiment, struts 16 extend in a three-dimensional manner between intersections 18 above and below the plane of the paper, favoring no particular plane. Thus, any given strut 16 may extend from an intersection 18 in any direction relative to other struts 16 that join at that intersection 18. Struts 16 and intersections 18 may have generally curved shapes and define between them a multitude of pores 20 or interstitial spaces in solid phase 12. Struts 16 and intersections 18 form an interconnected, continuous solid phase.

As illustrated in FIG. 1, the structural components of the solid phase 12 of elastomeric matrix 10, namely struts 16 and intersections 18, may appear to have a somewhat laminar configuration as though some were cut from a single sheet, it will be understood that this appearance may in part be attributed to the difficulties of representing complex three-dimensional structures in a two dimensional figure. Struts 16 and intersections 18 may have, and in many cases will have, non-laminar shapes including circular, elliptical and non-circular cross-sectional shapes and cross sections that may vary in area along the particular structure, for example, they may taper to smaller and/or larger cross sections while traversing along their longest dimension.

The cells of elastomeric matrix 10 are formed from clusters or groups of pores 20, which would form the walls of a cell except that the cell walls 22 of most of the pores 20 are absent or substantially absent owing to reticulation. In particular, a small number of pores 20 may have a cell wall of structural material also called a “window” or “window pane” such as cell wall 22. Such cell walls are undesirable to the extent that they obstruct the passage of fluid and/or propagation and proliferation of tissues through pores 20. Cell walls 22 may, in one embodiment, be removed in a suitable process step, such as reticulation as discussed below.

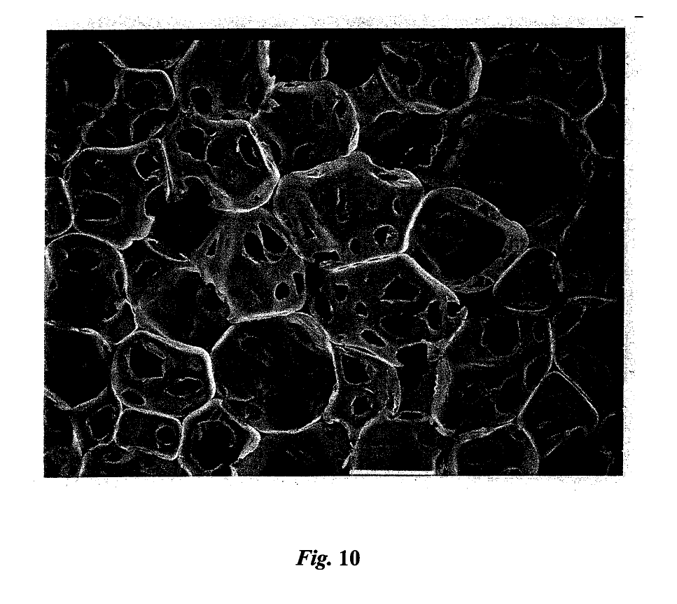

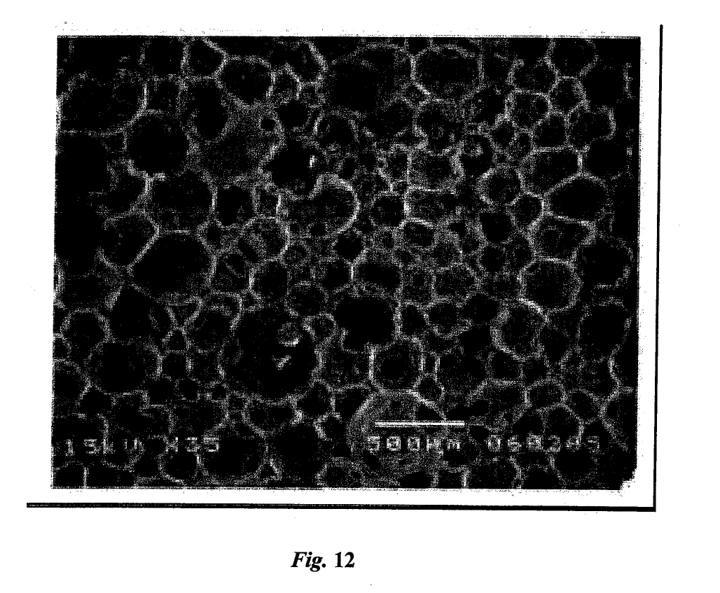

The individual cells forming the reticulated elastomeric matrix are characterized by their average cell diameter or, for nonspeherical cells, by their largest transverse dimension. The reticulated elastomeric matrix comprises a network of cells that form a three-dimensional spatial structure or void phase 14 which is interconnected via the open pores 20 therein. In one embodiment, the cells form a 3-dimensional superstructure. In FIGS. 10 and 12, the boundaries of individual cells can be visualized from the white-appearing sectioned struts 16 and/or intersections 18. The pores 20 are generally two- or three-dimensional structures. The pores provide connectivity between the individual cells, or between clusters or groups of pores which form a cell.

Except for boundary terminations at the macrostructural surface, in the embodiment shown in FIG. 1 solid phase 12 of elastomeric matrix 10 comprises few, if any, free-ended, dead-ended or projecting “strut-like” structures extending from struts 16 or intersections 18 but not connected to another strut or intersection.

However, in an alternative embodiment, solid phase 12 can be provided with a plurality of such fibrils (not shown), e.g., from about 1 to about 5 fibrils per strut 16 or intersection 18. In some applications, such fibrils may be useful, for example, for the additional surface area they provide.

Struts 16 and intersections 18 can be considered to define the shape and configuration of the pores 20 that make up void phase 14 (or vice versa). Many of pores 20, in so far as they may be discretely identified, open into and communicate, by the at least partial absence of cell walls 22, with at least two other pores 20. At intersections 18, three or more pores 20 may be considered to meet and intercommunicate. In certain embodiments, void phase 14 is continuous or substantially continuous throughout elastomeric matrix 10, meaning that there are few if any closed cell pores. Such closed cell pores, the interior volume of each of which has no communication with any other cell, e.g., is isolated from an adjacent cells by cell walls 22, represent loss of useful volume and may obstruct access of useful fluids to interior strut and intersection structures 16 and 18 of elastomeric matrix 10.

In one embodiment, closed cell pores, if present, comprise less than about 90% of the volume of elastomeric matrix 10. In another embodiment, closed cell pores, if present, comprise less than about 80% of the volume of elastomeric matrix 10. In another embodiment, closed cell pores, if present, comprise less than about 70% of the volume of elastomeric matrix 10. In another embodiment, closed cell pores, if present, comprise less than about 50% of the volume of elastomeric matrix 10. In another embodiment, closed cell pores, if present, comprise less than about 30% of the volume of elastomeric matrix 10. In another embodiment, closed cell pores, if present, comprise less than about 25% of the volume of elastomeric matrix 10. In another embodiment, closed cell pores, if present, comprise less than about 20% of the volume of elastomeric matrix 10. In another embodiment, closed cell pores, if present, comprise less than about 15% of the volume of elastomeric matrix 10. In another embodiment, closed cell pores, if present, comprise less than about 10% of the volume of elastomeric matrix 10. In another embodiment, closed cell pores, if present, comprise less than about 5% of the volume of elastomeric matrix 10. In another embodiment, closed cell pores, if present, comprise less than about 2% of the volume of elastomeric matrix 10. The presence of closed cell pores can be noted by their influence in reducing the volumetric flow rate of a fluid through elastomeric matrix 10 and/or as a reduction in cellular ingrowth and proliferation into elastomeric matrix 10.

In another embodiment, elastomeric matrix 10 is reticulated. In another embodiment, elastomeric matrix 10 is substantially reticulated. In another embodiment, elastomeric matrix 10 is fully reticulated. In another embodiment, elastomeric matrix 10 has many cell walls 22 removed. In another embodiment, elastomeric matrix 10 has most cell walls 22 removed. In another embodiment, elastomeric matrix 10 has substantially all cell walls 22 removed.

In another embodiment, solid phase 12, which may be described as reticulated, comprises a continuous network of solid structures, such as struts 16 and intersections 18, without any significant terminations, isolated zones or discontinuities, other than at the boundaries of the elastomeric matrix, in which network a hypothetical line may be traced entirely through the material of solid phase 12 from one point in the network to any other point in the network.

In another embodiment, void phase 14 is also a continuous network of interstitial spaces, or intercommunicating fluid passageways for gases or liquids, which fluid passageways extend throughout and are defined by (or define) the structure of solid phase 12 of elastomeric matrix 10 and open into all its exterior surfaces. In other embodiments, as described above, there are only a few, substantially no, or no occlusions or closed cell pores that do not communicate with at least one other pore 20 in the void network. Also in this void phase network, a hypothetical line may be traced entirely through void phase 14 from one point in the network to any other point in the network.

In concert with the objectives of the invention, in one embodiment the microstructure of elastomeric matrix 10 is constructed to permit or encourage cellular adhesion to the surfaces of solid phase 12, neointima formation thereon and cellular and tissue ingrowth and proliferation into pores 20 of void phase 14, when elastomeric matrix 10 resides in suitable in vivo locations for a period of time.

In another embodiment, such cellular or tissue ingrowth and proliferation, which may for some purposes include fibrosis, can occur or be encouraged not just into exterior layers of pores 20, but into the deepest interior of and throughout elastomeric matrix 10. Thus, in this embodiment, the space occupied by elastomeric matrix 10 becomes entirely filled by the cellular and tissue ingrowth and proliferation in the form of fibrotic, scar or other tissue except for the space occupied by the elastomeric solid phase 12. In another embodiment, the inventive implantable device functions so that ingrown tissue is kept vital, for example, by the prolonged presence of a supportive microvasculature.

To this end, particularly with regard to the morphology of void phase 14, in one embodiment elastomeric matrix 10 is reticulated with open interconnected pores. Without being bound by any particular theory, this is thought to permit natural irrigation of the interior of elastomeric matrix 10 with bodily fluids, e.g., blood, even after a cellular population has become resident in the interior of elastomeric matrix 10 so as to sustain that population by supplying nutrients thereto and removing waste products therefrom. In another embodiment, elastomeric matrix 10 is reticulated with open interconnected pores of a particular size range. In another embodiment, elastomeric matrix 10 is reticulated with open interconnected pores with a distribution of size ranges.

It is intended that the various physical and chemical parameters of elastomeric matrix 10 including in particular the parameters to be described below, be selected to encourage cellular ingrowth and proliferation according to the particular application for which an elastomeric matrix 10 is intended.

It will be understood that such constructions of elastomeric matrix 10 that provide interior cellular irrigation will be fluid permeable and may also provide fluid access through and to the interior of the matrix for purposes other than cellular irrigation, for example, for elution of pharmaceutically-active agents, e.g., a drug, or other biologically useful materials. Such materials may optionally be secured to the interior surfaces of elastomeric matrix 10.

In another embodiment of the invention, gaseous phase 12 can be filled or contacted with a deliverable treatment gas, for example, a sterilant such as ozone or a wound healant such as nitric oxide, provided that the macrostructural surfaces are sealed, for example by a bioabsorbable membrane to contain the gas within the implanted product until the membrane erodes releasing the gas to provide a local therapeutic or other effect.

Useful embodiments of the invention include structures that are somewhat randomized, as shown in FIG. 1 where the shapes and sizes of struts 16, intersections 18 and pores 20 vary substantially, and more ordered structures which also exhibit the described features of three-dimensional interpenetration of solid and void phases, structural complexity and high fluid permeability. Such more ordered structures can be produced by the processes of the invention as will be further described below.

Porosity

Post-reticulation, void phase 14 may comprise as little as 10% by volume of elastomeric matrix 10, referring to the volume provided by the interstitial spaces of elastomeric matrix 10 before any optional interior pore surface coating or layering is applied, such as for a reticulated elastomeric matrix that, after reticulation, has been compressively molded and/or reinforced as described in detail herein. In another embodiment, void phase 14 may comprise as little as 20% by volume of elastomeric matrix 10. In another embodiment, void phase 14 may comprise as little as 35% by volume of elastomeric matrix 10. In another embodiment, void phase 14 may comprise as little as 50% by volume of elastomeric matrix 10. In one embodiment, the volume of void phase 14, as just defined, is from about 10% to about 99% of the volume of elastomeric matrix 10. In another embodiment, the volume of void phase 14, as just defined, is from about 20% to about 99% of the volume of elastomeric matrix 10. In another embodiment, the volume of void phase 14, as just defined, is from about 30% to about 97% of the volume of elastomeric matrix 10. In another embodiment, the volume of void phase 14, as just defined, is from about 50% to about 99% of the volume of elastomeric matrix 10. In another embodiment, the volume of void phase 14, as just defined, is from about 70% to about 99% of the volume of elastomeric matrix 10. In another embodiment, the volume of void phase 14 is from about 80% to about 98% of the volume of elastomeric matrix 10. In another embodiment, the volume of void phase 14 is from about 90% to about 98% of the volume of elastomeric matrix 10.

As used herein, when a pore is spherical or substantially spherical, its largest transverse dimension is equivalent to the diameter of the pore. When a pore is non-spherical, for example, ellipsoidal or tetrahedral, its largest transverse dimension is equivalent to the greatest distance within the pore from one pore surface to another, e.g., the major axis length for an ellipsoidal pore or the length of the longest side for a tetrahedral pore. As used herein, the “average diameter or other largest transverse dimension” refers to the number average diameter, for spherical or substantially spherical pores, or to the number average largest transverse dimension, for non-spherical pores.

In one embodiment relating to orthopedic applications and the like, to encourage cellular ingrowth and proliferation and to provide adequate fluid permeability, the average diameter or other largest transverse dimension of pores 20 is at least about 10 μm. In another embodiment, the average diameter or other largest transverse dimension of pores 20 is at least about 20 μm. In another embodiment, the average diameter or other largest transverse dimension of pores 20 is at least about 50 μm. In another embodiment, the average diameter or other largest transverse dimension of pores 20 is at least about 100 μm. In another embodiment, the average diameter or other largest transverse dimension of pores 20 is at least about 150 μm. In another embodiment, the average diameter or other largest transverse dimension of pores 20 is at least about 250 μm. In another embodiment, the average diameter or other largest transverse dimension of pores 20 is greater than about 250 μm. In another embodiment, the average diameter or other largest transverse dimension of pores 20 is greater than 250 μm. In another embodiment, the average diameter or other largest transverse dimension of pores 20 is at least about 450 μm. In another embodiment, the average diameter or other largest transverse dimension of pores 20 is greater than about 450 μm. In another embodiment, the average diameter or other largest transverse dimension of pores 20 is greater than 450 μm. In another embodiment, the average diameter or other largest transverse dimension of pores 20 is at least about 500 μm.

In another embodiment relating to orthopedic applications and the like, the average diameter or other largest transverse dimension of pores 20 is not greater than about 600 μm. In another embodiment, the average diameter or other largest transverse dimension of pores 20 is not greater than about 500 μm. In another embodiment, the average diameter or other largest transverse dimension of pores 20 is not greater than about 450 μm. In another embodiment, the average diameter or other largest transverse dimension of pores 20 is not greater than about 350 μm. In another embodiment, the average diameter or other largest transverse dimension of pores 20 is not greater than about 250 μm. In another embodiment, the average diameter or other largest transverse dimension of pores 20 is not greater than about 150 μm. In another embodiment, the average diameter or other largest transverse dimension of pores 20 is not greater than about 20 μm.

In another embodiment relating to orthopedic applications and the like, the average diameter or other largest transverse dimension of pores 20 is from about 10 μm to about 50 μm. In another embodiment, the average diameter or other largest transverse dimension of pores 20 is from about 20 μm to about 150 μm. In another embodiment, the average diameter or other largest transverse dimension of pores 20 is from about 150 μm to about 250 μm. In another embodiment, the average diameter or other largest transverse dimension of pores 20 is from about 250 μm to about 500 μm. In another embodiment, the average diameter or other largest transverse dimension of pores 20 is from about 450 μm to about 600 μm. In another embodiment, the average diameter or other largest transverse dimension of pores 20 is from about 10 μm to about 500 μm. In another embodiment, the average diameter or other largest transverse dimension of pores 20 is from about 20 μm to about 600 μm. In another embodiment, the average diameter or other largest transverse dimension of pores 20 is from about 50 μm to about 600 μm. In another embodiment, the average diameter or other largest transverse dimension of pores 20 is from about 100 μm to about 500 μm. In another embodiment, the average diameter or other largest transverse dimension of pores 20 is from about 150 μm to about 350 μm.

In one embodiment relating to orthopedic applications and the like, to encourage cellular ingrowth and proliferation and to provide adequate fluid permeability, the average diameter or other largest transverse dimension of the cells of elastomeric matrix 10 is at least about 100 μm. In another embodiment, the average diameter or other largest transverse dimension of it cells is at least about 150 μm. In another embodiment, the average diameter or other largest transverse dimension of it cells is at least about 200 μm. In another embodiment, the average diameter or other largest transverse dimension of it cells is at least about 250 μm.

In another embodiment relating to orthopedic applications and the like, the average diameter or other largest transverse dimension of the cells of elastomeric matrix 10 is not greater than about 1000 μm. In another embodiment, the average diameter or other largest transverse dimension of its cells is not greater than about 850 μm. In another embodiment, the average diameter or other largest transverse dimension of its cells is not greater than about 450 μm. In another embodiment, the average diameter or other largest transverse dimension of its cells is not greater than about 700 μm. In another embodiment, the average diameter or other largest transverse dimension of its cells is not greater than about 650 μm.

In another embodiment relating to orthopedic applications and the like, the average diameter or other largest transverse dimension of the cells of elastomeric matrix 10 is from about 100 μm to about 1000 μm. In another embodiment, the average diameter or other largest transverse dimension of its cells is from about 150 μm to about 850 μm. In another embodiment, the average diameter or other largest transverse dimension of its cells is from about 200 μm to about 700 μm. In another embodiment, the average diameter or other largest transverse dimension of its cells is from about 250 μm to about 650 μm.

In another embodiment, an implantable device made from elastomeric matrix 10 may comprise pore sizes that vary from small, e.g., 20 μm, to large, e.g., 500 μm, in a single device. In another embodiment, an implantable device made from elastomeric matrix 10 may comprise cell sizes that vary from small, e.g., 100 μm, to large, e.g., 1000 μm, in a single device. In another embodiment, such a variation may occur across the cross-section of the entire material or across any sub-section of a cross-section. In another embodiment, such a variation occurs in a systematic gradual transition. In another embodiment, such a variation occurs in a stepwise manner. For example, the pore size distribution can be from about 20 μm to about 70 μm on one end of an implantable device and be from about 300 μm to about 500 μm on another end of the device. This change in pore size distribution can take place in one or more continuous transitions or in one or more discrete steps. Such variations in pore size distribution result in continuous transition zones or in discrete steps, i.e., the transition from one pore size distribution to another may be more gradual in the case of a continuous transition or transitions but more distinct in the case of a discrete step or steps. With regard to pore orientation, similar transitions may occur in the orientation of the pores, with more oriented pores transitioning into less oriented pores or even into pores substantially devoid of orientation across the cross-section or across a sub-section of the cross-section. The difference in the pore size distribution and/or orientation of the pores across a cross-section of implantable devices made from elastomeric matrix 10 may allow the device to be engineered for preferential behavior in terms of cell type, cell attachment, cell ingrowth and/or cell proliferation. Alternatively, different pore size distribution and/or orientation of the pores across the cross-section of implantable devices made from elastomeric matrix 10 may allow the device to be engineered for preferential behavior in terms of tissue type, tissue attachment, tissue ingrowth and/or tissue proliferation.

It is well known that cells will adhere, proliferate and differentiate along and through the contours of the structure formed by the pore size distribution. The cell orientation and cell morphology will result in engineered or newly-formed tissue that may substantially replicate or mimic the anatomical features of real tissues, e.g., of the tissues being replaced. This preferential cell morphology and orientation ascribed to the continuous or step-wise pore size distribution variations, with or without pore orientation, can occur when the implantable device is placed, without prior cell seeding, into the tissue repair and regeneration site. This preferential cell morphology and orientation ascribed to the continuous or step-wise pore size distribution can also occur when the implantable device is placed into a patient, e.g., human or animal, tissue repair and regeneration site after being subjected to in vitro cell culturing. These continuous or step-wise pore size distribution variations, with or without pore orientation, can be important characteristics for TE scaffolds in a number of orthopedic applications, especially in soft tissue attachment, repair, regeneration, augmentation and/or support encompassing the spine, shoulder, knee, hand or joints, and in the growth of a prosthetic organ.

Size and Shape

Elastomeric matrix 10 can be readily fabricated in any desired size and shape. It is a benefit of the invention that elastomeric matrix 10 is suitable for mass production from bulk stock by subdividing such bulk stock, e.g., by cutting, die punching, laser slicing, or compression molding. In one embodiment, subdividing the bulk stock can be done using a heated surface. It is a further benefit of the invention that the shape and configuration of elastomeric matrix 10 may vary widely and can readily be adapted to desired anatomical morphologies.

The size, shape, configuration and other related details of elastomeric matrix 10 can be either customized to a particular application or patient or standardized for mass production. However, economic considerations favor standardization. To this end, elastomeric matrix 10 can be embodied in a kit comprising elastomeric implantable device pieces of different sizes and shapes. Also, as discussed elsewhere in the present specification and as is disclosed in the applications to which priority is claimed, multiple, e.g. two, three or four, individual elastomeric matrices 10 can be used as an implantable device system for a single target biological site, being sized or shaped or both sized and shaped to function cooperatively for treatment of an individual target site.

The practitioner performing the procedure, who may be a surgeon or other medical or veterinary practitioner, researcher or the like, may then choose one or more implantable devices from the available range to use for a specific treatment, for example, as is described in the applications to which priority is claimed.

By way of example, the minimum dimension of elastomeric matrix 10 may be as little as 0.5 mm and the maximum dimension as much as 100 mm or even greater. However, in one embodiment it is contemplated that an elastomeric matrix 10 of such dimension intended for implantation would have an elongated shape, such as the shapes of cylinders, rods, tubes or elongated prismatic forms, or a folded, coiled, helical or other more compact configuration. Comparably, a dimension as small as 0.5 mm can be a transverse dimension of an elongated shape or of a ribbon or sheet-like implantable device.

In an alternative embodiment, an elastomeric matrix 10 having a spherical, cubical, tetrahedral, toroidal or other form having no dimension substantially elongated when compared to any other dimension and with a diameter or other maximum dimension of from about 0.5 mm to about 500 mm may have utility, for example, for an orthopedic application site. In another embodiment, the elastomeric matrix 10 having such a form has a diameter or other maximum dimension from about 3 mm to about 20 mm.

For most implantable device applications, macrostructural sizes of elastomeric matrix 10 include the following embodiments: compact shapes such as spheres, cubes, pyramids, tetrahedrons, cones, cylinders, trapezoids, parallelepipeds, ellipsoids, fusiforms, tubes or sleeves, and many less regular shapes having transverse dimensions of from about 1 mm to about 200 mm (In another embodiment, these transverse dimensions are from about 5 mm to about 100 mm.); and sheet- or strip-like shapes having a thickness of from about 0.5 to about 20 mm (In another embodiment, these thickness are from about 1 to about 5 mm.) and lateral dimensions of from about 5 to about 200 mm (In another embodiment, these, lateral dimensions are from about 10 to about 100 mm.).

For treatment of orthopedic applications, it is an advantage of the invention that the implantable elastomeric matrix elements can be effectively employed without any need to closely conform to the configuration of the orthopedic application site, which may often be complex and difficult to model. Thus, in one embodiment, the implantable elastomeric matrix elements of the invention have significantly different and simpler configurations, for example, as described in the applications to which priority is claimed.

Furthermore, in one embodiment, the implantable device of the present invention, or implantable devices if more than one is used, should not completely fill the orthopedic application site even when fully expanded in situ. In one embodiment, the fully expanded implantable device(s) of the present invention are smaller in a dimension than the orthopedic application site and provide sufficient space within the orthopedic application site to ensure vascularization, cellular ingrowth and proliferation, and for possible passage of blood to the implantable device. In another embodiment, the fully expanded implantable device(s) of the present invention are substantially the same in a dimension as the orthopedic application site. In another embodiment, the fully expanded implantable device(s) of the present invention are larger in a dimension than the orthopedic application site. In another embodiment, the fully expanded implantable device(s) of the present invention are smaller in volume than the orthopedic application site. In another embodiment, the fully expanded implantable device(s) of the present invention are substantially the same volume as orthopedic application site. In another embodiment, the fully expanded implantable device(s) of the present invention are larger in volume than the orthopedic application site. In another embodiment, after being placed in the orthopedic application site the expanded implantable device(s) of the present invention may swell, e.g., by up to 1-20% in one dimension in one embodiment, by up to 1-30% in one dimension in another embodiment, or by up to 1-40% in one dimension in another embodiment, by absorption and/or adsorption of water or other body fluids.

Some useful implantable device shapes may approximate the contour of a portion of the target orthopedic application site. In one embodiment, the implantable device is shaped as relatively simple convex, dish-like or hemispherical or hemi-ellipsoidal shape and size that is appropriate for treating multiple different sites in different patients.

It is contemplated, in another embodiment, that upon implantation, before their pores become filled with biological fluids, bodily fluids and/or tissue, such implantable devices for orthopedic applications and the like do not entirely fill, cover or span the biological site in which they reside and that an individual implanted elastomeric matrix 10 will, in many cases although not necessarily, have at least one dimension of no more than 50% of the biological site within the entrance thereto or over 50% of the damaged tissue that is being repaired or replaced. In another embodiment, an individual implanted elastomeric matrix 10 as described above will have at least one dimension of no more than 75% of the biological site within the entrance thereto or over 75% of the damaged tissue that is being repaired or replaced. In another embodiment, an individual implanted elastomeric matrix 10 as described above will have at least one dimension of no more than 95% of the biological site within the entrance thereto or over 95% of the damaged tissue that is being repaired or replaced.

In another embodiment, that upon implantation, before their pores become filled with biological fluids, bodily fluids and/or tissue, such implantable devices for orthopedic applications and the like substantially fill, cover or span the biological site in which they reside and an individual implanted elastomeric matrix 10 will, in many cases, although not necessarily, have at least one dimension of no more than about 100% of the biological site within the entrance thereto or cover 100% of the damaged tissue that is being repaired or replaced. In another embodiment, an individual implanted elastomeric matrix 10 as described above will have at least one dimension of no more than about 98% of the biological site within the entrance thereto or cover 98% of the damaged tissue that is being repaired or replaced. In another embodiment, an individual implanted elastomeric matrix 10 as described above will have at least one dimension of no more than about 102% of the biological site within the entrance thereto or cover 102% of the damaged tissue that is being repaired or replaced.

In another embodiment, that upon implantation, before their pores become filled with biological fluids, bodily fluids and/or tissue, such implantable devices for orthopedic applications and the like over fill, cover or span the biological site in which they reside and an individual implanted elastomeric matrix 10 will, in many cases, although not necessarily, have at least one dimension of more than about 105% of the biological site within the entrance thereto or cover 105% of the damaged tissue that is being repaired or replaced. In another embodiment, an individual implanted elastomeric matrix 10 as described above will have at least one dimension of more than about 125% of the biological site within the entrance thereto or cover 125% of the damaged tissue that is being repaired or replaced. In another embodiment, an individual implanted elastomeric matrix 10 as described above will have at least one dimension of more than about 150% of the biological site within the entrance thereto or cover 150% of the damaged tissue that is being repaired or replaced. In another embodiment, an individual implanted elastomeric matrix 10 as described above will have at least one dimension of more than about 200% of the biological site within the entrance thereto or cover 200% of the damaged tissue that is being repaired or replaced. In another embodiment, an individual implanted elastomeric matrix 10 as described above will have at least one dimension of more than about 300% of the biological site within the entrance thereto or cover 300% of the damaged tissue that is being repaired or replaced.

One embodiment for use in the practice of the invention is a reticulated elastomeric matrix 10 which is sufficiently flexible and resilient, i.e., resiliently-compressible, to enable it to be initially compressed under ambient conditions, e.g., at 25° C., from a relaxed configuration to a first, compact configuration for delivery via a delivery-device, e.g., catheter, endoscope, syringe, cystoscope, trocar or other suitable introducer instrument, for delivery in vitro and, thereafter, to expand to a second, working configuration in situ. Furthermore, in another embodiment, an elastomeric matrix has the herein described resilient-compressibility after being compressed about 5-95% of an original dimension (e.g., compressed about 19/20th- 1/20th of an original dimension). In another embodiment, an elastomeric matrix has the herein described resilient-compressibility after being compressed about 10-90% of an original dimension (e.g., compressed about 9/10th- 1/10th of an original dimension). As used herein, elastomeric matrix 10 has “resilient-compressibility”, i.e., is “resiliently-compressible”, when the second, working configuration, in vitro, is at least about 50% of the size of the relaxed configuration in at least one dimension. In another embodiment, the resilient-compressibility of elastomeric matrix 10 is such that the second, working configuration, in vitro, is at least about 80% of the size of the relaxed configuration in at least one dimension. In another embodiment, the resilient-compressibility of elastomeric matrix 10 is such that the second, working configuration, in vitro, is at least about 90% of the size of the relaxed configuration in at least one dimension. In another embodiment, the resilient-compressibility of elastomeric matrix 10 is such that the second, working configuration, in vitro, is at least about 97% of the size of the relaxed configuration in at least one dimension.

In another embodiment, an elastomeric matrix has the herein described resilient-compressibility after being compressed about 5-95% of its original volume (e.g., compressed about 19/20th- 1/20th of its original volume). In another embodiment, an elastomeric matrix has the herein described resilient-compressibility after being compressed about 10-90% of its original volume (e.g., compressed about 9/10th- 1/10th of its original volume). As used herein, “volume” is the volume swept-out by the outermost 3-dimensional contour of the elastomeric matrix. In another embodiment, the resilient-compressibility of elastomeric matrix 10 is such that the second, working configuration, in vivo, is at least about 50% of the volume occupied by the relaxed configuration. In another embodiment, the resilient-compressibility of elastomeric matrix 10 is such that the second, working configuration, in vivo, is at least about 80% of the volume occupied by the relaxed configuration. In another embodiment, the resilient-compressibility of elastomeric matrix 10 is such that the second, working configuration, in vivo, is at least about 90% of the volume occupied by the relaxed configuration. In another embodiment, the resilient-compressibility of elastomeric matrix 10 is such that the second, working configuration, in vivo, occupies at least about 97% of the volume occupied by the elastomeric matrix in its relaxed configuration.

Well-Characterized Elastomers and Elastomeric Implantable Devices

Elastomers for use as the structural material of elastomeric matrix 10 alone or in combination in blends or solutions are, in one embodiment, well-characterized synthetic elastomeric polymers having suitable mechanical properties which have been sufficiently characterized with regard to chemical, physical or biological properties as to be considered biodurable and suitable for use as in vivo implantable devices in patients, particularly in mammals and especially in humans. In another embodiment, elastomers for use as the structural material of elastomeric matrix 10 are sufficiently characterized with regard to chemical, physical and biological properties as to be considered biodurable and suitable for use as in vivo implantable devices in patients, particularly in mammals and especially in humans.

Elastomeric Matrix Physical Properties

Elastomeric matrix 10, a reticulated elastomeric matrix, an implantable device comprising a reticulated elastomeric matrix, and/or an implantable device comprising a compressive molded reticulated elastomeric matrix can have any suitable bulk density, also known as specific gravity, consistent with its other properties. For example, in one embodiment, the bulk density, as measured pursuant to the test method described in ASTM Standard D3574, may be from about 0.005 g/cc to about 0.96 g/cc (from about 0.31 lb/ft³ to about 60 lb/ft³). In another embodiment, the bulk density may be from about 0.048 g/cc to about 0.56 g/cc (from about 3.0 lb/ft³ to about 35 lb/ft³). In another embodiment, the bulk density may be from about 0.005 g/cc to about 0.15 g/cc (from about 0.31 lb/ft³ to about 9.4 lb/ft³). In another embodiment, the bulk density may be from about 0.008 g/cc to about 0.127 g/cc (from about 0.5 lb/ft³ to about 8 lb/ft³). In another embodiment, the bulk density may be from about 0.015 g/cc to about 0.115 g/cc (from about 0.93 lb/ft³ to about 7.2 lb/ft³). In another embodiment, the bulk density may be from about 0.024 g/cc to about 0.104 g/cc (from about 1.5 lb/ft³ to about 6.5 lb/ft³).

Elastomeric matrix 10 can have any suitable microscopic surface area consistent with its other properties. Those skilled in the art, e.g., from an exposed plane of the porous material, can routinely estimate the microscopic surface area from the pore frequency, e.g., the number of pores per linear millimeter, and can routinely estimate the pore frequency from the average cell side diameter in μm.

Other suitable physical properties will be apparent to, or will become apparent to, those skilled in the art.

Elastomeric Matrix Mechanical Properties

In one embodiment, reticulated elastomeric matrix 10 has sufficient structural integrity to be self-supporting and free-standing in vitro. However, in another embodiment, elastomeric matrix 10 can be furnished with structural supports such as ribs or struts.

The reticulated elastomeric matrix 10 has sufficient tensile strength such that it can withstand normal manual or mechanical handling during its intended application and during post-processing steps that may be required or desired without tearing, breaking, crumbling, fragmenting or otherwise disintegrating, shedding pieces or particles, or otherwise losing its structural integrity. The tensile strength of the starting material(s) should not be so high as to interfere with the fabrication or other processing of elastomeric matrix 10.

Thus, for example, in one embodiment reticulated elastomeric matrix 10 may have a tensile strength of from about 700 kg/m² to about 350,000 kg/m² (from about 1 psi to about 500 psi). In another embodiment, elastomeric matrix 10 may have a tensile strength of from about 700 kg/m² to about 70,000 kg/m² (from about 1 psi to about 100 psi). In another embodiment, reticulated elastomeric matrix 10 may have a tensile modulus of from about 7,000 kg/m² to about 140,000 kg/m² (from about 10 psi to about 200 psi). In another embodiment, elastomeric matrix 10 may have a tensile modulus of from about 17,500 kg/m² to about 70,000 kg/m² (from about 25 psi to about 100 psi).

Sufficient ultimate tensile elongation is also desirable. For example, in another embodiment, reticulated elastomeric matrix 10 has an ultimate tensile elongation of at least about 25%. In another embodiment, elastomeric matrix 10 has an ultimate tensile elongation of at least about 200%.

In one embodiment, the elastomeric matrix 10 expands from the first, compact configuration to the second, working configuration over a short time, e.g., about 95% recovery in 90 seconds or less in one embodiment, or in 40 seconds or less in another embodiment, each from 75% compression strain held for up to 10 minutes. In another embodiment, the expansion from the first, compact configuration to the second, working configuration occurs over a short time, e.g., about 95% recovery in 180 seconds or less in one embodiment, in 90 seconds or less in another embodiment, in 60 seconds or less in another embodiment, each from 75% compression strain held for up to 30 minutes. In another embodiment, elastomeric matrix 10 recovers in about 10 minutes to occupy at least about 97% of the volume occupied by its relaxed configuration, following 75% compression strain held for up to 30 minutes.

In one embodiment, reticulated elastomeric matrix 10 may have a compressive modulus of from about 7,000 kg/m² to about 140,000 kg/m² (from about 10 psi to about 200 psi). In another embodiment, elastomeric matrix 10 may have a compressive modulus of from about 17,500 kg/m² to about 70,000 kg/m² (from about 25 psi to about 100 psi). In another embodiment, reticulated elastomeric matrix 10 has a compressive strength of from about 700 kg/m² to about 350,000 kg/m² (from about 1 psi to about 500 psi) at 50% compression strain. In another embodiment, reticulated elastomeric matrix 10 has a compressive strength of from about 700 kg/m² to about 70,000 kg/m² (from about 1 psi to about 100 psi) at 50% compression strain. In another embodiment, reticulated elastomeric matrix 10 has a compressive strength of from about 7,000 kg/m² to about 420,000 kg/m² (from about 10 psi to about 600 psi) at 75% compression strain. In another embodiment, reticulated elastomeric matrix 10 has a compressive strength of from about 7,000 kg/m² to about 140,000 kg/m² (from about 10 psi to about 200 psi) at 75% compression strain.

In another embodiment, reticulated elastomeric matrix 10 has a compression set, when compressed to 50% of its thickness at about 25° C., i.e., pursuant to ASTM D3574, of not more than about 30%. In another embodiment, elastomeric matrix 10 has a compression set of not more than about 20%. In another embodiment, elastomeric matrix 10 has a compression set of not more than about 10%. In another embodiment, elastomeric matrix 10 has a compression set of not more than about 5%.

In another embodiment, reticulated elastomeric matrix 10 has a tear strength, as measured pursuant to the test method described in ASTM Standard D3574, of from about 0.18 kg/linear cm to about 8.90 kg/linear cm (from about 1 lbs/linear inch to about 50 lbs/linear inch). In another embodiment, reticulated elastomeric matrix 10 has a tear strength, as measured pursuant to the test method described in ASTM Standard D3574, of from about 0.18 kg/linear cm to about 1.78 kg/linear cm (from about 1 lbs/linear inch to about 10 lbs/linear inch).

In another embodiment, reticulated elastomeric matrix 10 has a static recovery time, t-90%, as measured pursuant to the test method described in Example 5, of from about 50 sec. to about 2,500 sec. In another embodiment, reticulated elastomeric matrix 10 has a static recovery time, t-90%, of from about 100 sec. to about 2,000 sec. In another embodiment, reticulated elastomeric matrix 10 has a static recovery time, t-90%, of from about 125 sec. to about 1,500 sec.

In another embodiment, reticulated elastomeric matrix 10 has a dynamic recovery time, t-90%, as measured after 5,000 cycles at a frequency of 1 Hz in air pursuant to the test method described in Example 5, of from about 5 sec. to about 200 sec. In another embodiment, reticulated elastomeric matrix 10 has a dynamic recovery time, t-90%, as measured after 100,000 cycles at a frequency of 1 Hz in air, of less than about 4,000 sec. in one embodiment, less than about 1,750 sec. in another embodiment, less than about 200 sec. in another embodiment, or from about 50 sec. to about 4,000 sec. in another embodiment. In another embodiment, reticulated elastomeric matrix 10 has a dynamic recovery time, t-90%, as measured after 100,000 cycles at a frequency of 1 Hz in water, of less than about 3,000 sec. in one embodiment, less than about 1,500 sec. in another embodiment, less than about 100 sec. in another embodiment, or from about 50 sec. to about 3,000 sec. in another embodiment.

Table 1 summarizes mechanical property and other properties applicable to embodiments of reticulated elastomeric matrix 10 including those reticulated elastomeric matrices that have been annealed after reticulation. Additional suitable mechanical properties will be apparent to, or will become apparent to, those skilled in the art.

TABLE 1
Properties of Reticulated Elastomeric Matrix 10

| Property | Typical Values |
|---|---|
| Specific Gravity/Bulk Density | 0.31-9.4 lb/ft³ (0.005-0.15 g/cc) |
| Tensile Modulus | 10-200 psi (7,000-140,000 kg/m²) |
| Tensile Strength | 1-500 psi (700-350,000 kg/m²) |
| Ultimate Tensile Elongation | ≧25% |
| Compressive Modulus | 10-200 psi (7,000-140,000 kg/m²) |
| Compressive Strength at 50% | 1-500 psi (700-350,000 kg/m²) |
| Compression | |
| Compressive Strength at 75% | 10-600 psi (7,000-420,000 kg/m²) |
| Compression | |
| 50% Compression Set, 22 hours | ≦30% |
| at 25° C. | |
| Tear Strength | 1-50 lbs/linear inch |
| | (0.18-8.90 kg/linear cm) |
| Static Recovery Time [t-90% | 50-2,500 |
| (sec) after 50% Uniaxial | |
| Compression for 120 minutes] | |
| Dynamic Recovery Time [t-90% | |
| (sec) after no. of Cycles | |
| at 50% Compression ± 5% Strain | |
| at 1 Hz:] | |
| 5,000 cycles (in air) | 5-200 |
| 100,000 cycles (in air) | 50-4,000 |
| 100,000 cycles (in water) | 50-3,000 |

The mechanical properties of the porous materials described herein, if not indicated otherwise, may be determined according to ASTM D3574-01 entitled “Standard Test Methods for Flexible Cellular Materials—Slab, Bonded and Molded Urethane Foams”, or other such method as is known to be appropriate by those skilled in the art.

Furthermore, if porosity is to be imparted to the elastomer employed for elastomeric matrix 10 after rather than during the polymerization reaction, good processability is also desirable for post-polymerization shaping and fabrication. For example, in one embodiment, elastomeric matrix 10 has low tackiness.

Biodurability and Biocompatibility

In one embodiment, elastomers are sufficiently biodurable so as to be suitable for long-term implantation in patients, e.g., animals or humans. Biodurable elastomers and elastomeric matrices have chemical, physical and/or biological properties so as to provide a reasonable expectation of biodurability, meaning that the elastomers will continue to exhibit stability when implanted in an animal, e.g., a mammal, for a period of at least 29 days. The intended period of long-term implantation may vary according to the particular application. For many applications, substantially longer periods of implantation may be required and for such applications biodurability for periods of at least 6, 12 or 24 months or 5 years, or longer, may be desirable. Of especial benefit are elastomers that may be considered biodurable for the life of a patient. In the case of the possible use of an embodiment of elastomeric matrix 10 to treat, e.g., a spinal column deficiency, because such conditions may present themselves in rather young human patients, perhaps in their thirties, biodurability in excess of 50 years may be advantageous.

In another embodiment, the period of implantation will be at least sufficient for cellular ingrowth and proliferation to commence, for example, in at least about 4-8 weeks. In another embodiment, elastomers are sufficiently well characterized to be suitable for long-term implantation by having been shown to have such chemical, physical and/or biological properties as to provide a reasonable expectation of biodurability, meaning that the elastomers will continue to exhibit biodurability when implanted for extended periods of time.

Without being bound by any particular theory, biodurability of the elastomeric matrix formed by a process comprising polymerization, cross-linking, foaming and reticulation include the selection of starting components that are biodurable and the stoichiometric ratios of those components, such that the elastomeric matrix retains the biodurability of its components. For example, elastomeric matrix biodurability can be promoted by minimizing the presence and formation of chemical bonds and groups, such as ester groups, that are susceptible to hydrolysis, e.g., at the patient's body fluid temperature and pH. As a further example, a curing step in excess of about 2 hours can be performed after cross-linking and foaming to minimize the presence of free amine groups in the elastomeric matrix. Moreover, it is important to minimize degradation that can occur during the elastomeric matrix preparation process, e.g., because of exposure to shearing or thermal energy such as may occur during admixing, dissolution, cross-linking and/or foaming, by ways known to those in the art.

As previously discussed, biodurable elastomers and elastomeric matrices are stable for extended periods of time in a biological environment. Such products do not exhibit significant symptoms of breakdown, degradation, erosion or significant deterioration of mechanical properties relevant to their use when exposed to biological environments and/or bodily stresses for periods of time commensurate with that use. However, some amount of cracking, fissuring or a loss in toughness and stiffening—at times referred to as ESC or environmental stress cracking—may not be relevant to many orthopedic and other uses as described herein. Many in vivo applications, e.g., when elastomeric matrix 10 is used for treatment at an orthopedic application site, expose it to little, if any, mechanical stress and, thus, are unlikely to result in mechanical failure leading to serious patient consequences. Accordingly, the absence of ESC may not be a prerequisite for biodurability of suitable elastomers in such applications for which the present invention is intended because elastomeric properties become less important as endothielozation, encapsulation and cellular ingrowth and proliferation advance.

Furthermore, in certain implantation applications, it is anticipated that elastomeric matrix 10 will become in the course of time, for example, in 2 weeks to 1 year, walled-off or encapsulated by tissue, scar tissue or the like, or incorporated and totally integrated or bio-integrated into, e.g., the tissue being repaired or the lumen being treated. In this condition, elastomeric matrix 10 has reduced exposure to mobile or circulating biological fluids. Accordingly, the probabilities of biochemical degradation or release of undesired, possibly nocuous, products into the host organism may be attenuated if not eliminated.

In one embodiment, the elastomeric matrix has good biodurability accompanied by good biocompatibility such that the elastomer induces few, if any, adverse reactions in vivo. To that end, in another embodiment for use in the invention are elastomers or other materials that are free of biologically undesirable or hazardous substances or structures that can induce such adverse reactions or effects in vivo when lodged in an intended site of implantation for the intended period of implantation. Such elastomers accordingly should either entirely lack or should contain only very low, biologically tolerable quantities of cytotoxins, mutagens, carcinogens and/or teratogens. In another embodiment, biological characteristics for biodurability of elastomers to be used for fabrication of elastomeric matrix 10 include at least one of resistance to biological degradation, and absence of or extremely low: cytotoxicity, hemotoxicity, carcinogenicity, mutagenicity, or teratogenicity.

Elastomeric Matrices from Elastomer Polymerization, Cross-Linking and Foaming

In further embodiments, the invention provides a porous biodurable elastomer and a process for polymerizing, cross-linking and foaming the same which can be used to produce a biodurable reticulated elastomeric matrix 10 as described herein. In another embodiment, reticulation follows.

More particularly, in another embodiment, the invention provides a process for preparing a biodurable elastomeric polyurethane matrix which comprises synthesizing the matrix from a polycarbonate polyol component and an isocyanate component by polymerization, cross-linking and foaming, thereby forming pores, followed by reticulation of the foam to provide a reticulated product. The product is designated as a polycarbonate polyurethane, being a polymer comprising urethane groups formed from, e.g., the hydroxyl groups of the polycarbonate polyol component and the isocyanate groups of the isocyanate component. In this embodiment, the process employs controlled chemistry to provide a reticulated elastomer product with good biodurability characteristics. Pursuant to the invention, the polymerization is conducted to provide a foam product employing chemistry that avoids biologically undesirable or nocuous constituents therein.

In one embodiment, as one starting material, the process employs at least one polyol component. For the purposes of this application, the term “polyol component” includes molecules comprising, on the average, about 2 hydroxyl groups per molecule, i.e., a difunctional polyol or a diol, as well as those molecules comprising, on the average, greater than about 2 hydroxyl groups per molecule, i.e., a polyol or a multi-functional polyol. Exemplary polyols can comprise, on the average, from about 2 to about 5 hydroxyl groups per molecule. In one embodiment, as one starting material, the process employs a difunctional polyol component. In this embodiment, because the hydroxyl group functionality of the diol is about 2, it does not provide the so-called “soft segment” with soft segment cross-linking. In another embodiment, as one starting material of the polyol component, the process employs a multi-functional polyol component in sufficient quantity to provide a controlled degree of soft segment cross-linking. In another embodiment, the process provides sufficient soft segment cross-linking to yield a stable foam. In another embodiment, the soft segment is composed of a polyol component that is generally of a relatively low molecular weight, in one embodiment from about 350 to about 6,000 Daltons, and from about 450 to about 4,000 Daltons in another embodiment. Thus, these polyols are generally liquids or low-melting-point solids. This soft segment polyol is terminated with hydroxyl groups, either primary or secondary. In another embodiment, a soft segment polyol component has about 2 hydroxyl groups per molecule. In another embodiment, a soft segment polyol component has greater than about 2 hydroxyl groups per molecule; more than 2 hydroxyl groups per polyol molecule are required of some polyol molecules to impart soft-segment cross-linking.

In one embodiment, the average number of hydroxyl groups per molecule in the polyol component is about 2. In another embodiment, the average number of hydroxyl groups per molecule in the polyol component is greater than about 2. In another embodiment, the average number of hydroxyl groups per molecule in the polyol component is greater than 2. In one embodiment, the polyol component comprises a tertiary carbon linkage. In one embodiment, the polyol component comprises a plurality of tertiary carbon linkages.

In one embodiment, the polyol component is a polyether polyol, polyester polyol, polycarbonate polyol, hydrocarbon polyol, polysiloxane polyol, poly(ether-co-ester) polyol, poly(ether-co-carbonate) polyol, poly(ether-co-hydrocarbon) polyol, poly(ether-co-siloxane) polyol, poly(ester-co-carbonate) polyol, poly(ester-co-hydrocarbon) polyol, poly(ester-co-siloxane) polyol, poly(carbonate-co-hydrocarbon) polyol, poly(carbonate-co-siloxane) polyol, poly(hydrocarbon-co-siloxane) polyol, or a mixture thereof.

Polyether-type polyols are oligomers of, e.g., alkylene oxides such as ethylene oxide or propylene oxide, polymerized with glycols or polyhydric alcohols, the latter to result in hydroxyl functionalities greater than 2 to allow for soft segment cross-linking. Polyester-type polyols are oligomers of, e.g., the reaction product of a carboxylic acid with a glycol or triol, such as ethylene glycol adipate, propylene glycol adipate, butylene glycol adipate, diethylene glycol adipate, phthalates, polycaprolactone and castor oil. When the reactants include those with hydroxyl functionalities greater than 2, e.g., polyhydric alcohols, soft segment cross-linking is possible.

Polycarbonate-type polyols typically result from the reaction, with a carbonate monomer, of one type of hydrocarbon diol or, for a plurality of diols, hydrocarbon diols each with a different hydrocarbon chain length between the hydroxyl groups. The length of the hydrocarbon chain between adjacent carbonates is the same as the hydrocarbon chain length of the original diol(s). For example, a difunctional polycarbonate polyol can be made by reacting 1,6-hexanediol with a carbonate, such as sodium hydrogen carbonate, to provide the polycarbonate-type polyol 1,6-hexanediol carbonate. The molecular weight for the commercial-available products of this reaction varies from about 500 to about 5,000 Daltons. If the polycarbonate polyol is a solid at 25° C., it is typically melted prior to further processing. Alternatively, in one embodiment, a liquid polycarbonate polyol component can prepared from a mixture of hydrocarbon diols, e.g., all three or any binary combination of 1,6-hexanediol, cyclohexyl dimethanol and 1,4-butanediol. Without being bound by any particular theory, such a mixture of hydrocarbon diols is thought to break-up the crystallinity of the product polycarbonate polyol component, rendering it a liquid at 25° C., and thereby, in foams comprising it, yield a relatively softer foam.

When the reactants used to produce the polycarbonate polyol include those with hydroxyl functionalities greater than 2, e.g., polyhydric alcohols, soft segment cross-linking is possible. Polycarbonate polyols with an average number of hydroxyl groups per molecule greater than 2, e.g., a polycarbonate triol, can be made by using, for example, hexane triol, in the preparation of the polycarbonate polyol component. To make a liquid polycarbonate triol component, mixtures with other hydroxyl-comprising materials, for example, cyclohexyl trimethanol and/or butanetriol, can be reacted with the carbonate along with the hexane triol.

Commercial hydrocarbon-type polyols typically result from the free-radical polymerization of dienes with vinyl monomers, therefore, they are typically difunctional hydroxyl-terminated materials.

Polysiloxane polyols are oligomers of, e.g., alkyl and/or aryl substituted siloxanes such as dimethyl siloxane, diphenyl siloxane or methyl phenyl siloxane, comprising hydroxyl end-groups. Polysiloxane polyols with an average number of hydroxyl groups per molecule greater than 2, e.g., a polysiloxane triol, can be made by using, for example, methyl hydroxymethyl siloxane, in the preparation of the polysiloxane polyol component.

A particular type of polyol need not be limited to those formed from a single monomeric unit. For example, a polyether-type polyol can be formed from a mixture of ethylene oxide and propylene oxide.

Additionally, in another embodiment, copolymers or copolyols can be formed from any of the above polyols by methods known to those in the art. Thus, the following binary component polyol copolymers can be used: poly(ether-co-ester) polyol, poly(ether-co-carbonate) polyol, poly(ether-co-hydrocarbon) polyol, poly(ether-co-siloxane) polyol, poly(ester-co-carbonate) polyol, poly(ester-co-hydrocarbon) polyol, poly(ester-co-siloxane) polyol, poly(carbonate-co-hydrocarbon) polyol, poly(carbonate-co-siloxane) polyol and poly(hydrocarbon-co-siloxane) polyol. For example, a poly(ether-co-ester) polyol can be formed from units of polyethers formed from ethylene oxide copolymerized with units of polyester comprising ethylene glycol adipate. In another embodiment, the copolymer is a poly(ether-co-carbonate) polyol, poly(ether-co-hydrocarbon) polyol, poly(ether-co-siloxane) polyol, poly(carbonate-co-hydrocarbon) polyol, poly(carbonate-co-siloxane) polyol, poly(hydrocarbon-co-siloxane) polyol or a mixture thereof. In another embodiment, the copolymer is a poly(carbonate-co-hydrocarbon) polyol, poly(carbonate-co-siloxane) polyol, poly(hydrocarbon-co-siloxane) polyol or a mixture thereof. In another embodiment, the copolymer is a poly(carbonate-co-hydrocarbon) polyol. For example, a poly(carbonate-co-hydrocarbon) polyol can be formed by polymerizing 1,6-hexanediol, 1,4-butanediol and a hydrocarbon-type polyol with carbonate.

In another embodiment, the polyol component is a polyether polyol, polycarbonate polyol, hydrocarbon polyol, polysiloxane polyol, poly(ether-co-carbonate) polyol, poly(ether-co-hydrocarbon) polyol, poly(ether-co-siloxane) polyol, poly(carbonate-co-hydrocarbon) polyol, poly(carbonate-co-siloxane) polyol, poly(hydrocarbon-co-siloxane) polyol or a mixture thereof. In another embodiment, the polyol component is a polycarbonate polyol, hydrocarbon polyol, polysiloxane polyol, poly(carbonate-co-hydrocarbon) polyol, poly(carbonate-co-siloxane) polyol, poly(hydrocarbon-co-siloxane) polyol or a mixture thereof. In another embodiment, the polyol component is a polycarbonate polyol, poly(carbonate-co-hydrocarbon) polyol, poly(carbonate-co-siloxane) polyol, poly(hydrocarbon-co-siloxane) polyol or a mixture thereof. In another embodiment, the polyol component is a polycarbonate polyol, poly(carbonate-co-hydrocarbon) polyol, poly(carbonate-co-siloxane) polyol or a mixture thereof. In another embodiment, the polyol component is a polycarbonate polyol.

Furthermore, in another embodiment, mixtures, admixtures and/or blends of polyols and copolyols can be used in the elastomeric matrix of the present invention. In another embodiment, the molecular weight of the polyol is varied. In another embodiment, the functionality of the polyol is varied.

In another embodiment, as either difunctional polycarbonate polyols or difunctional hydrocarbon polyols cannot, on their own, induce soft segment cross-linking, higher functionality is introduced into the formulation through the use of a chain extender component with a hydroxyl group functionality greater than about 2. In another embodiment, higher functionality is introduced through the use of an isocyanate component with an isocyanate group functionality greater than about 2.

Commercial polycarbonate diols with molecular weights of from about 500 to about 5,000 Daltons, such as POLY-CD CD220 from Arch Chemicals, Inc. (Norwalk, Conn.) and PC-1733 from Stahl USA, Inc. (Peabody, Mass.), are readily available. Commercial hydrocarbon polyols are available from Sartomer (Exton, Pa.). Commercial polyether polyols are readily available, such as the PLURACOL, e.g., PLURACOL GP430 with functionality of 3 and LUPRANOL lines from BASF Corp. (Wyandotte, Mich.), VORANOL from Dow Chemical Corp. (Midland, Mich.), BAYCOLL B, DESMOPHEN and MULTRANOL from Bayer Corp. (Leverkusen, Germany), and from Huntsman Corp. (Madison Heights, Mich.). Commercial polyester polyols are readily available, such as LUPRAPHEN from BASF, TONE polycaprolactone and VORANOL from Dow, BAYCOLL A and the DESMOPHEN U series from Bayer, and from Huntsman. Commercial polysiloxane polyols are readily available, such as from Dow.

The process also employs at least one isocyanate component and, optionally, at least one chain extender component to provide the so-called “hard segment”. For the purposes of this application, the term “isocyanate component” includes molecules comprising, on the average, about 2 isocyanate groups per molecule as well as those molecules comprising, on the average, greater than about 2 isocyanate groups per molecule. The isocyanate groups of the isocyanate component are reactive with reactive hydrogen groups of the other ingredients, e.g., with hydrogen bonded to oxygen in hydroxyl groups and with hydrogen bonded to nitrogen in amine groups of the polyol component, chain extender, cross-linker and/or water.

In one embodiment, the average number of isocyanate groups per molecule in the isocyanate component is about 2. In another embodiment, the average number of isocyanate groups per molecule in the isocyanate component is greater than about 2. In another embodiment, the average number of isocyanate groups per molecule in the isocyanate component is greater than 2.

The isocyanate index, a quantity well known to those in the art, is the mole ratio of the number of isocyanate groups in a formulation available for reaction to the number of groups in the formulation that are able to react with those isocyanate groups, e.g., the reactive groups of diol(s), polyol component(s), chain extender(s) and water, when present. In one embodiment, the isocyanate index is from about 0.9 to about 1.1. In another embodiment, the isocyanate index is from about 0.9 to about 1.02. In another embodiment, the isocyanate index is from about 0.98 to about 1.02. In another embodiment, the isocyanate index is from about 0.9 to about 1.0. In another embodiment, the isocyanate index is from about 0.9 to about 0.98.

Exemplary diisocyanates include aliphatic diisocyanates, isocyanates comprising aromatic groups, the so-called “aromatic diisocyanates”, or a mixture thereof. Aliphatic diisocyanates include tetramethylene diisocyanate, cyclohexane-1,2-diisocyanate, cyclohexane-1,4-diisocyanate, hexamethylene diisocyanate, isophorone diisocyanate, methylene-bis-(p-cyclohexyl isocyanate) (“H₁₂ MDI”), or a mixture thereof. Aromatic diisocyanates include p-phenylene diisocyanate, 4,4′-diphenylmethane diisocyanate (“4,4′-MDI”), 2,4′-diphenylmethane diisocyanate (“2,4′-MDI”), 2,4-toluene diisocyanate (“2,4-TDI”), 2,6-toluene diisocyanate (“2,6-TDI”), m-tetramethylxylene diisocyanate, or a mixture thereof.

Exemplary isocyanate components comprising, on the average, greater than about 2 isocyanate groups per molecule, include an adduct of hexamethylene diisocyanate and water comprising about 3 isocyanate groups, available commercially as DESMODUR N100 from Bayer, and a trimer of hexamethylene diisocyanate comprising about 3 isocyanate groups, available commercially as MONDUR N3390 from Bayer.

In one embodiment, the isocyanate component contains a mixture of at least about 5% by weight of 2,4′-MDI with the balance 4,4′-MDI. In another embodiment, the isocyanate component contains a mixture of at least 5% by weight of 2,4′-MDI with the balance 4,4′-MDI. In another embodiment, the isocyanate component contains a mixture of from about 5% to about 50% by weight of 2,4′-MDI with the balance 4,4′-MDI. In another embodiment, the isocyanate component contains a mixture of from 5% to about 50% by weight of 2,4′-MDI with the balance 4,4′-MDI. In another embodiment, the isocyanate component contains a mixture of from about 5% to about 40% by weight of 2,4′-MDI with the balance 4,4′-MDI. In another embodiment, the isocyanate component contains a mixture of from 5% to about 40% by weight of 2,4′-MDI with the balance 4,4′-MDI. In another embodiment, the isocyanate component contains a mixture of from 5% to about 35% by weight of 2,4′-MDI with the balance 4,4′-MDI. Without being bound by any particular theory, it is thought that the use of higher amounts of 2,4′-MDI in a blend with 4,4′-MDI results in a softer elastomeric matrix because of the disruption of the crystallinity of the hard segment arising out of the asymmetric 2,4′-MDI structure.

Suitable diisocyanates include MDI, such as ISONATE 125M, certain members of the PAPI series from Dow and ISONATE 50 OP from Dow; isocyanates containing a mixture of 4,4′-MDI and 2,4′-MDI, such as RUBINATE 9433 and RUBINATE 9258, each from Huntsman, and MONDUR MRS 2 and MRS 20 from Bayer; TDI, e.g., from Lyondell Corp. (Houston, Tex.); isophorone diisocyanate, such as VESTAMAT from Degussa (Germany); H₁₂ MDI, such as DESMODUR W from Bayer; and various diisocyanates from BASF.

Suitable isocyanate components comprising, on the average, greater than about 2 isocyanate groups per molecule, include the following modified diphenylmethane-diisocyanate type, each available from Dow: ISOBIND 1088, with an isocyanate group functionality of about 3; ISONATE 143L, with an isocyanate group functionality of about 2.1; PAPI 27, with an isocyanate group functionality of about 2.7; PAPI 94, with an isocyanate group functionality of about 2.3; PAPI 580N, with an isocyanate group functionality of about 3; and PAPI 20, with an isocyanate group functionality of about 3.2.

Exemplary chain extenders include diols, diamines, alkanol amines or a mixture thereof. In one embodiment, the chain extender is an aliphatic diol having from 2 to 10 carbon atoms. In another embodiment, the diol chain extender is selected from ethylene glycol, 1,2-propane diol, 1,3-propane diol, 1,4-butane diol, 1,5-pentane diol, diethylene glycol, triethylene glycol or a mixture thereof. In another embodiment, the chain extender is a diamine having from 2 to 10 carbon atoms. In another embodiment, the diamine chain extender is selected from ethylene diamine, 1,3-diaminobutane, 1,4-diaminobutane, 1,5 diaminopentane, 1,6-diaminohexane, 1,7-diaminoheptane, 1,8-diaminooctane, isophorone diamine or a mixture thereof. In another embodiment, the chain extender is an alkanol amine having from 2 to 10 carbon atoms. In another embodiment, the alkanol amine chain extender is selected from diethanolamine, triethanolamine, isopropanolamine, dimethylethanolamine, methyldiethanolamine, diethylethanolamine or a mixture thereof.

Commercially available chain extenders include the JEFFAMINE series of diamines, triamines and polyetheramines available from Huntsman, VERSAMIN isophorone diamine from Creanova, the VERSALINK series of diamines available from Air Products Corp. (Allentown, Pa.), ethanolamine, diethylethanolamine and isopropanolamine available from Dow, and various chain extenders from Bayer, BASF and UOP Corp. (Des Plaines, Ill.).

In one embodiment, a small quantity of an optional ingredient, such as a multi-functional hydroxyl compound or other cross-linker having a functionality greater than 2, e.g., glycerol, is present to allow cross-linking. In another embodiment, the optional multi-functional cross-linker is present in an amount just sufficient to achieve a stable foam, i.e., a foam that does not collapse to become non-foamlike. Alternatively, or in addition, polyfunctional adducts of aliphatic and cycloaliphatic isocyanates can be used to impart cross-linking in combination with aromatic diisocyanates. Alternatively, or in addition, polyfunctional adducts of aliphatic and cycloaliphatic isocyanates can be used to impart cross-linking in combination with aliphatic diisocyanates.

Optionally, the process employs at least one catalyst in certain embodiments selected from a blowing catalyst, e.g., a tertiary amine, a gelling catalyst, e.g., dibutyltin dilaurate, or a mixture thereof. Moreover, it is known in the art that tertiary amine catalysts can also have gelling effects, that is, they can act as a blowing and gelling catalyst. Exemplary tertiary amine catalysts include the TOTYCAT line from Toyo Soda Co. (Japan), the TEXACAT line from Texaco Chemical Co. (Austin, Tex.), the KOSMOS and TEGO lines from Th. Goldschmidt Co. (Germany), the DMP line from Rohm and Haas (Philadelphia, Pa.), the KAO LIZER line from Kao Corp. (Japan), and the QUINCAT line from Enterprise Chemical Co. (Altamonte Springs, Fla.). Exemplary organotin catalysts include the FOMREZ and FOMREZ UL lines from Witco Corporation (Middlebury, Conn.), the COCURE and COSCAT lines from Cosan Chemical Co. (Carlstadt, N.J.), and the DABCO and POLYCAT lines from Air Products.

In certain embodiments, the process employs at least one surfactant. Exemplary surfactants include TEGOSTAB BF 2370, B-8300, B-8305 and B-5055, all from Goldschmidt, D.C. 5241 from Dow Corning (Midland, Mich.), and other non-ionic organosilicones, such as the polydimethylsiloxane types available from Dow Corning, Air Products and General Electric (Waterford, N.Y.).

In certain embodiments, the process employs at least one cell-opener. Exemplary cell-openers include ORTEGOL 501 from Goldschmidt.)

Cross-linked polyurethanes may be prepared by approaches which include the prepolymer process and the one-shot process. An embodiment involving a prepolymer is as follows. First, the prepolymer is prepared by a conventional method from at least one isocyanate component (e.g., MDI) and at least one multi-functional soft segment material with a functionality greater than 2 (e.g., a polyether-based soft segment with a functionality of 3). Then, the prepolymer, optionally at least one catalyst (e.g., dibutyltin dilaurate) and at least one difunctional chain extender (e.g., 1,4-butanediol) are admixed in a mixing vessel to cure or cross-link the mixture. In another embodiment, cross-linking takes place in a mold. In another embodiment, cross-linking and foaming, i.e., pore formation, take place together. In another embodiment, cross-linking and foaming take place together in a mold.

Alternatively, the so-called “one-shot” approach may be used. A one-shot embodiment requires no separate prepolymer-making step. In one embodiment, the starting materials, such as those described in the previous paragraph, are admixed in a mixing vessel and then foamed and cross-linked. In another embodiment, the ingredients are heated before they are admixed. In another embodiment, the ingredients are heated as they are admixed. In another embodiment, cross-linking takes place in a mold. In another embodiment, foaming and cross-linking take place together. In another embodiment, cross-linking and foaming take place together in a mold. In another embodiment, all of the ingredients except for the isocyanate component are admixed in a mixing vessel. The isocyanate component is then added, e.g., with high-speed stirring, and cross-linking and foaming ensue. In another embodiment, this foaming mix is poured into a mold and allowed to rise.

In another embodiment, the polyol component is admixed with the isocyanate component and other optional additives, such as a viscosity modifier, surfactant and/or cell opener, to form a first liquid. In another embodiment, the polyol component is a liquid at the mixing temperature. In another embodiment, the polyol component is a solid, therefore, the mixing temperature is raised such that the polyol component is liquefied prior to mixing, e.g., by heating. Next, a second liquid is formed by admixing a blowing agent and optional additives, such as gelling catalyst and/or blowing catalyst. Then, the first liquid and the second liquid are admixed in a mixing vessel and then foamed and cross-linked.

In another embodiment, any or all of the processing approaches of the invention may be used to make foam with a density greater than 3.4 lbs/ft³ (0.054 g/cc). In this embodiment, cross-linker(s), such as glycerol, are used; the functionality of the isocyanate component is from 2.0 to 2.4; the isocyanate component consists essentially of MDI; and the amount of 4,4′-MDI is greater than about 50% by weight of the isocyanate component. The molecular weight of the polyol component is from about 1,000 to about 2,000 Daltons. The amount of blowing agent, e.g., water, is adjusted to obtain non-reticulated foam densities greater than 3.4 lbs/ft³ (0.054 g/cc). A reduced amount of blowing agent may reduce the number of urea linkages in the material. Any reduction in stiffness and/or tensile strength and/or compressive strength caused by fewer urea linkages can be compensated for by using di-functional chain extenders, such as butanediol, and/or increasing the density of the foam, and/or by increasing the amount of cross-linking agent used. In one embodiment, reducing the degree of cross-linking and, consequently, increasing the foam's toughness and/or elongation to break should allow for more efficient reticulation. In another embodiment, the higher density foam material which results can better withstand the sudden impact of one or a plurality of reticulation steps, e.g., two reticulation steps, and can provide for minimal, if any, damage to struts 16.

In one embodiment, the invention provides a process for preparing a flexible polyurethane biodurable matrix capable of being reticulated based on polycarbonate polyol component and isocyanate component starting materials. In another embodiment, a porous biodurable elastomer polymerization process for making a resilient polyurethane matrix is provided which process comprises admixing a polycarbonate polyol component and an aliphatic isocyanate component, for example H₁₂ MDI.

In another embodiment, the foam is substantially free of isocyanurate linkages. In another embodiment, the foam has no isocyanurate linkages. In another embodiment, the foam is substantially free of biuret linkages. In another embodiment, the foam has no biuret linkages. In another embodiment, the foam is substantially free of allophanate linkages. In another embodiment, the foam has no allophanate linkages. In another embodiment, the foam is substantially free of isocyanurate and biuret linkages. In another embodiment, the foam has no isocyanurate and biuret linkages. In another embodiment, the foam is substantially free of isocyanurate and allophanate linkages. In another embodiment, the foam has no isocyanurate and allophanate linkages. In another embodiment, the foam is substantially free of allophanate and biuret linkages. In another embodiment, the foam has no allophanate and biuret linkages. In another embodiment, the foam is substantially free of allophanate, biuret and isocyanurate linkages. In another embodiment, the foam has no allophanate, biuret and isocyanurate linkages. Without being bound by any particular theory, it is thought that the absence of allophanate, biuret and/or isocyanurate linkages provides an enhanced degree of flexibility to the elastomeric matrix because of lower cross-linking of the hard segments.

In certain embodiments, additives helpful in achieving a stable foam, for example, surfactants and catalysts, can be included. By limiting the quantities of such additives to the minimum desirable while maintaining the functionality of each additive, the impact on the toxicity of the product can be controlled.

In one embodiment, elastomeric matrices of various densities, e.g., from about 0.005 to about 0.15 g/cc (from about 0.31 to about 9.4 lb/ft³) are produced. The density is controlled by, e.g., the amount of blowing or foaming agent, the isocyanate index, the isocyanate component content in the formulation, the reaction exotherm, and/or the pressure of the foaming environment.

Exemplary blowing agents include water and the physical blowing agents, e.g., volatile organic chemicals such as hydrocarbons, ethanol and acetone, and various fluorocarbons and their more environmentally friendly replacements, such as hydrofluorocarbons, chlorofluorocarbons and hydrochlorofluorocarbons. The reaction of water with an isocyanate group yields carbon dioxide, which serves as a blowing agent. Moreover, combinations of blowing agents, such as water with a fluorocarbon, can be used in certain embodiments. In another embodiment, water is used as the blowing agent. Commercial fluorocarbon blowing agents are available from Huntsman, E.I. duPont de Nemours and Co. (Wilmington, Del.), Allied Chemical (Minneapolis, Minn.) and Honeywell (Morristown, N.J.).

For the purpose of this invention, for every 100 parts by weight (or 100 grams) of polyol component (e.g., polycarbonate polyol, polysiloxane polyol) used to make an elastomeric matrix through foaming and cross-linking, the amounts of the other components present, by weight, in a formulation are as follows: from about 10 to about 90 parts (or grams) isocyanate component (e.g., MDIs, their mixtures, H₁₂MDI) with an isocyanate index of from about 0.85 to about 1.10, from about 0.5 to about 6.0 parts (or grams) blowing agent (e.g., water), from about 0.1 to about 2.0 parts (or grams) blowing catalyst (e.g., tertiary amine), from about 0.1 to about 8.0 parts (or grams) surfactant, and from about 0.1 to about 8.0 parts (or grams) cell opener. Of course, the actual amount of isocyanate component used is related to and depends upon the magnitude of the isocyanate index for a particular formulation. Additionally, for every 100 parts by weight (or 100 grams) of polyol component used to make an elastomeric matrix through foaming and cross-linking, the amounts of the following optional components, when present in a formulation, are as follows by weight: up to about 20 parts (or grams) chain extender, up to about 20 parts (or grams) cross-linker, up to about 0.5 parts (or grams) gelling catalyst (e.g., a compound comprising tin), up to about 10.0 parts (or grams) physical blowing agent (e.g., hydrocarbons, ethanol, acetone, fluorocarbons), and up to about 15 parts (or grams) viscosity modifier.

In other embodiments, for every 100 parts by weight (or 100 grams) of polyol component (e.g., polycarbonate polyol, polysiloxane polyol) used to make an elastomeric matrix through foaming and cross-linking, the amounts of the other components present, by weight, in a formulation are as follows: from about 10 to about 90 parts (or grams) isocyanate component (e.g., MDIs, their mixtures, H₁₂MDI) with an isocyanate index of from about 0.85 to about 1.2 in one embodiment, from about 0.85 to about 1.019 in another embodiment, from about 0.5 to about 6.0 parts (or grams) blowing agent (e.g., water), optionally, from about 0.05 to about 3.0 parts (or grams) catalyst (e.g., tertiary amine), such as a blowing catalyst and/or gelling catalyst, from about 0.1 to about 8.0 parts (or grams) surfactant, optionally, from about 0.1 to about 8.0 parts (or grams) cell opener, optionally, from about 0.05 to about 8.0 parts (or grams) cross-linking agent, e.g., glycerine, and optionally, from about 0.05 to about 8.0 parts (or grams) chain extender, e.g., 1,4-butanediol.

Matrices with appropriate properties for the purposes of the invention, as determined by testing, for example, acceptable compression set at human body temperature, airflow, tensile strength and compressive properties, can then be reticulated.

In another embodiment, the gelling catalyst, e.g., the tin catalyst, is omitted and optionally substituted with another catalyst, e.g., a tertiary amine. In one embodiment, the tertiary amine catalyst comprises one or more non-aromatic amines. In another embodiment, the reaction is conducted so that the tertiary amine catalyst, if employed, is wholly reacted into the polymer, and residues of same are avoided. In another embodiment, the gelling catalyst is omitted and, instead, higher foaming temperatures are used.

In another embodiment, to enhance biodurability and biocompatibility, ingredients for the polymerization process are selected so as to avoid or minimize the presence in the end product elastomeric matrix of biologically adverse substances or substances susceptible to biological attack.

An alternative preparation embodiment pursuant to the invention involves partial or total replacement of water as a blowing agent with water-soluble spheres, fillers or particles which are removed, e.g., by washing, extraction or melting, after full cross-linking of the matrix.

Further Process Aspects of the Invention

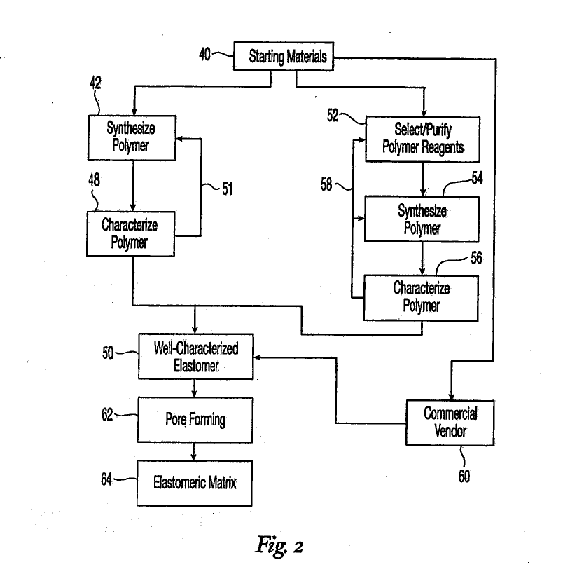

Referring now to FIG. 2, the schematic block flow diagram shown gives a broad overview of alternative embodiments of processes according to the invention whereby an implantable device comprising a biodurable, porous, reticulated, elastomeric matrix 10 can be prepared from raw elastomer or elastomer reagents by one or another of several different process routes.

In a first route, elastomers prepared by a process according to the invention, as described herein, are rendered to comprise a plurality of cells by using, e.g., a blowing agent or agents, employed during their preparation. In particular, starting materials 40, which may comprise, for example, a polyol component, an isocyanate, optionally a cross-linker, and any desired additives such as surfactants and the like, are employed to synthesize the desired elastomeric polymer, in synthesis step 42, either with or without significant foaming or other pore-generating activity. The starting materials are selected to provide desirable mechanical properties and to enhance biocompatibility and biodurability. The elastomeric polymer product of step 42 is then characterized, in step 48, as to chemical nature and purity, physical and mechanical properties and, optionally, also as to biological characteristics, all as described above, yielding well-characterized elastomer 50. Optionally, the characterization data can be employed to control or modify step 42 to enhance the process or the product, as indicated by pathway 51.

Alternately, well-characterized elastomer 50 is generated from starting materials 40 and supplied to the process facility by a commercial vendor 60. Such elastomers are synthesized pursuant to known methods and subsequently rendered porous. Exemplary elastomers of this type are BIONATE 80A aromatic polycarbonate-urethane elastomer (from Polymer Technology Group Inc., Berkeley, Calif.), CARBOTHANE PC 3575A aliphatic polyurethane elastomer (Noveon Inc., Cleveland, Ohio), CARBOSIL silicone polycarbonate urethane (from Polymer Technology Group), BIOSPAN segmented polyurethane (from Polymer Technology Group), and CHRONOFLEX AL and CHRONOFLEX C (from CardioTech International Inc., Wilmington, Mass.). The elastomer 50 can be rendered porous, e.g., by a blowing agent employed in a polymerization reaction or in a post-polymerization step. In the post-polymerization step (e.g., starting with a commercially available exemplary elastomer or elastomers) a blowing agents or agents can enter the starting material(s), e.g., by absorbtion therein and/or adsorption thereon, optionally under the influence of elevated temperature and/or pressure, before the blowing gas is released from the blowing agent(s) to form an elastomeric matrix comprising pores. In one embodiment, the pores are interconnected. The amount of interconnectivity can depend on, e.g., the temperature applied to the polymer, the pressure applied to the polymer, the gas concentration in the polymer, the gas concentration on the polymer surface, the rate of gas release, and/or the mode of gas release.

If desired, the elastomeric polymer reagents employed in starting material 40 may be selected to avoid adverse by-products or residuals and purified, if necessary, in step 52. Polymer synthesis, step 54, is then conducted on the selected and purified starting materials and is conducted to avoid generation of adverse by-products or residuals. The elastomeric polymer produced in step 54 is then characterized, in step 56, as described previously for step 48, to facilitate production of a high quality, well-defined product, well-characterized elastomer 50. In another embodiment, the characterization results are fed back for process control as indicated by pathway 58 to facilitate production of a high quality, well-defined product, well-characterized elastomer 50.

The invention provides, in one embodiment, a reticulated biodurable elastomeric matrix comprising polymeric elements which are specifically designed for the purpose of biomedical implantation. The elastomeric matrix comprises biodurable polymeric materials and is prepared by a process or processes which avoid chemically changing the polymer, the formation of undesirable by-products, and residuals comprising undesirable unreacted starting materials. In some cases, foams comprising polyurethanes and created by known techniques may not be appropriate for long-term endovascular, orthopedic and related applications because of, e.g., the presence of undesirable unreacted starting materials or undesirable by-products. In one embodiment, the elastomeric matrix is formed from commercially available biodurable polymeric elastomeric material(s) and chemical change to the starting elastomeric material(s) is avoided in the process or processes by which the porous and reticulated elastomeric matrix is formed.

In another embodiment, chemical characteristics for biodurability of elastomers to be used for fabrication of elastomeric matrix 10 include one or more of: good oxidative stability; a chemistry that is free or substantially free of linkages that are prone to biological degradation, for example, certain polyether linkages or hydrolyzable ester linkages that may be introduced by incorporating a polyether or polyester polyol component into the polyurethane; a chemically well-defined product which is relatively refined or purified and free or substantially free of adverse impurities, reactants, by-products; oligomers and the like; a well-defined molecular weight, unless the elastomer is cross-linked; and solubility in a biocompatible solvent unless, of course, the elastomer is cross-linked.

In another embodiment, process-related characteristics, referring to a process used for the preparation of the elastomer of the solid phase 12, for biodurability of elastomers to be used for fabrication of elastomeric matrix 10 include one or more of: process reproducibility; process control for product consistency; and avoidance or substantial removal of adverse impurities, reactants, by-products, oligomers and the like.

The pore-making, reticulation and other post-polymerization processes of the invention discussed below are, in certain embodiments, carefully designed and controlled. To this end, in certain embodiments, processes of the invention avoid introducing undesirable residuals or otherwise adversely affecting the desirable biodurability properties of the starting material(s). In another embodiment, the starting material(s) may be further processed and/or characterized to enhance, provide or document a property relevant to biodurability. In another embodiment, the requisite properties of elastomers can be characterized as appropriate and the process features can be adapted or controlled to enhance biodurability, pursuant to the teachings of the present specification.

Formation of at Least Partially Reticulated Elastomeric Matrices by Microwave Irradiation

Another way to form an at least partially reticulated elastomeric matrix of the invention is through the use of microwave irradiation technology. In this process, 100 parts by weight of an elastomeric material, such as a polycarbonate urethane or a polycarbonate urethane urea, is used as the starting material, preferably provided in form of pellets or flakes. The elastomeric material is optionally admixed, e.g., blended, with from about 2 to about 70 parts by weight in one embodiment, from about 10 to about 35 parts by weight in another embodiment, of a more hydrophilic polymeric material such as poly(vinyl acetate) (PVA), poly(ethylene-co-vinyl acetate) (EVA), poly(vinyl alcohol) or any mixture thereof, using an appropriate melt blender or mixer, such as an extruder, twin-screw extruder or Brabender PLASTOGRAPH, to form a mixture. The blender or mixer can have a screw(s), paddle(s) or magnetic stirrer(s). In one embodiment, from about 0.1 to about 20 parts by weight, in another embodiment, from about 0.25 to about 5 parts by weight, of cross-linking agent is also added during admixing. In another embodiment, from about 1 to about 20 parts by weight, in another embodiment, from about 5 to about 15 parts by weight, of a blowing agent or agents is also added during admixing. In another embodiment, both a cross-linking agent and a blowing agent or agents are also added during admixing.

The resulting mixture can be heated in a sealed chamber using microwave irradiation generated at a frequency of from about 2.2 to about 6.0 Giga Hertz (GHz) in one embodiment, at about 2.45 GHz in another embodiment, or at about 5.8 GHz in another embodiment, to form a foamed at least partially reticulated elastomeric matrix structure with inter-connected and inter-communicating pores. Optionally, the mixture is also heated in the same sealed chamber in which it is microwave irradiated, e.g., by heating or convection heating, to a temperature of from about 70° C. to about 225° C. in one embodiment or from about 100° C. to about 180° C. in another embodiment to aid in the formation of a foamed at least partially reticulated elastomeric matrix structure with inter-connected and inter-communicating pores. Thus, if it is present, it is beneficial that the more hydrophilic polymeric material(s) be one(s) amenable to heating during microwave irradiation, thereby promoting the heating and foaming of the mixture comprising it. In one embodiment, the more hydrophilic polymeric material(s) is selected such that its dielectric loss and/or dielectric loss tangent is sufficiently great so that the more hydrophilic polymeric material is amenable to heating at the microwave irradiation frequency used.

This process can be either a batch process or a continuous process. Optionally, the elastomeric matrix formed can be further reticulated, as discussed below, to achieve the desired permeability.

According to other embodiments of the invention, the biodurable elastomeric material is selected from polycarbonate polyurethane urea, polycarbonate polyurea urethane, polycarbonate polyurethane, polycarbonate polysiloxane polyurethane, polycarbonatepolysiloxane polyurethane urea, polysiloxane polyurethane, polysiloxane polyurethane urea, polycarbonate hydrocarbon polyurethane, polycarbonate hydrocarbon polyurethane urea, or any mixture thereof. Of particular interest are thermoplastic elastomers such as polyurethanes whose chemistry is associated with good biodurability properties, for example. In one embodiment, such thermoplastic polyurethane elastomers include polycarbonate polyurethanes, polyester polyurethanes, polyether polyurethanes, polysiloxane polyurethanes, hydrocarbon polyurethanes (i.e., those thermoplastic elastomer polyurethanes formed from at least one isocyanate component comprising, on the average, about 2 isocyanate groups per molecule and at least one hydroxy-terminated hydrocarbon oligomer and/or hydrocarbon polymer), polyurethanes with so-called “mixed” soft segments, and mixtures thereof. Mixed soft segment polyurethanes are known to those skilled in the art and include, e.g., polycarbonate-polyester polyurethanes, polycarbonate-polyether polyurethanes, polycarbonate-polysiloxane polyurethanes, polycarbonate-hydrocarbon polyurethanes, polycarbonate-polysiloxane-hydrocarbon polyurethanes, polyester-polyether polyurethanes, polyester-polysiloxane polyurethanes, polyester-hydrocarbon polyurethanes, polyether-polysiloxane polyurethanes, polyether-hydrocarbon polyurethanes, polyether-polysiloxane-hydrocarbon polyurethanes and polysiloxane-hydrocarbon polyurethanes. In another embodiment, the thermoplastic polyurethane elastomer includes polycarbonate polyurethanes, polyether polyurethanes, polysiloxane polyurethanes, hydrocarbon polyurethanes, polyurethanes with these mixed soft segments, or mixtures thereof. In another embodiment, the thermoplastic polyurethane elastomer includes polycarbonate polyurethanes, polysiloxane polyurethanes, hydrocarbon polyurethanes, polyurethanes with these mixed soft segments, or mixtures thereof. In another embodiment, the thermoplastic polyurethane elastomer is a polycarbonate polyurethane, or mixtures thereof. In another embodiment, the thermoplastic polyurethane elastomer is a polysiloxane polyurethane, or mixtures thereof. In another embodiment, the thermoplastic polyurethane elastomer is a polysiloxane polyurethane, or mixtures thereof. In another embodiment, the thermoplastic polyurethane elastomer comprises at least one diisocyanate in the isocyanate component, at least one chain extender and at least one diol, and may be formed from any combination of the diisocyanates, difunctional chain extenders and diols described in detail above.

In one embodiment, the weight average molecular weight of the thermoplastic elastomer is from about 30,000 to about 500,000 Daltons. In another embodiment, the weight average molecular weight of the thermoplastic elastomer is from about 50,000 to about 250,000 Daltons.

Some suitable thermoplastics for practicing the invention, in one embodiment suitably characterized as described herein, can include: polyolefinic polymers with alternating secondary and quaternary carbons as described by Pinchuk et al. in U.S. Pat. No. 5,741,331 (and its divisional U.S. Pat. Nos. 6,102,939 and 6,197,240); block copolymers having an elastomeric block, e.g., a polyolefin, and a thermoplastic block, e.g., a styrene, as described by Pinchuk et al. in U.S. Patent Application Publication No. 2002/0107330 A1; thermoplastic segmented polyetherester, thermoplastic polydimethylsiloxane, di-block polystyrene polybutadiene, tri-block polystyrene polybutadiene, poly(acrylene ether sulfone)-poly(acryl carbonate) block copolymers, di-block copolymers of polybutadiene and polyisoprene, copolymers of ethylene vinyl acetate (EVA), segmented block co-polystyrene polyethylene oxide, di-block co-polystyrene polyethylene oxide, and tri-block co-polystyrene polyethylene oxide, e.g., as described by Penhasi in U.S. Patent Application Publication No. 2003/0208259 A1 (particularly, see paragraph [0035] therein); and polyurethanes with mixed soft segments comprising polysiloxane together with a polyether and/or a polycarbonate component, as described by Meijs et al. in U.S. Pat. No. 6,313,254; and those polyurethanes described by DiDomenico et al. in U.S. Pat. Nos. 6,149,678, 6,111,052 and 5,986,034. Also suitable for use in practicing the present invention are novel or known elastomers synthesized by a process according to the invention, as described herein. In another embodiment, an optional therapeutic agent may be loaded into the appropriate block of other elastomers used in the practice of the invention.

Some commercially-available thermoplastic elastomers suitable for use in practicing the present invention include the line of polycarbonate polyurethanes supplied under the trademark BIONATE by the Polymer Technology Group Inc. For example, the very well-characterized grades of polycarbonate polyurethane polymer BIONATE 80A, 55 and 90 are processable, reportedly have good mechanical properties, lack cytotoxicity, lack mutagenicity, lack carcinogenicity and are non-hemolytic. Another commercially-available elastomer suitable for use in practicing the present invention is the CHRONOFLEX C line of biodurable medical grade polycarbonate aromatic polyurethane thermoplastic elastomers available from CardioTech International, Inc. Yet another commercially-available elastomer suitable for use in practicing the present invention is the PELLETHANE line of thermoplastic polyurethane elastomers, in particular the 2363 series products and more particularly those products designated 81A and 85A, supplied by the Dow Chemical Company (Midland, Mich.). These commercial polyurethane polymers are linear, not cross-linked, polymers, therefore, they are readily analyzable and readily characterizable.

Reticulation of Elastomeric Matrices

Elastomeric matrix 10 can be subjected to any of a variety of post-processing treatments to enhance its utility, some of which are described herein and others of which will be apparent to those skilled in the art. In one embodiment, reticulation of an elastomeric matrix 10 of the invention, if not already a part of the described production process, may be used to remove at least a portion of any existing interior “windows”, i.e., the residual cell walls 22 illustrated in FIG. 1. Reticulation tends to increase porosity and fluid permeability.

Porous or foam materials with some ruptured cell walls are generally known as “open-cell” materials or foams. In contrast, porous materials known as “reticulated” or “at least partially reticulated” have many, i.e., at least about 40%, of the cell walls that would be present in an identical porous material except composed exclusively of cells that are closed, at least partially removed. Where the cell walls are least partially removed by reticulation, adjacent reticulated cells open into, interconnect with, and communicate with each other. Porous materials from which more, i.e., at least about 65%, of the cell walls have been removed are known as “further reticulated”. If most, i.e., at least about 80%, or substantially all, i.e., at least about 90%, of the cell walls have been removed then the porous material that remains is known as “substantially reticulated” or “fully reticulated”, respectfully. It will be understood that, pursuant to this art usage, a reticulated material or foam comprises a network of at least partially open interconnected cells.

“Reticulation” generally refers to a process for at least partially removing cell walls, not merely rupturing or tearing them by a crushing process. Moreover, crushing undesirable creates debris that must be removed by further processing. In another embodiment, the reticulation process substantially fully removes at least a portion of the cell walls. Reticulation may be effected, for example, by at least partially dissolving away cell walls, known variously as “solvent reticulation” or “chemical reticulation”; or by at least partially melting, burning and/or exploding out cell walls, known variously as “combustion reticulation”, “thermal reticulation” or “percussive reticulation”. Melted material arising from melted cell walls can be deposited on the struts. In one embodiment, such a procedure may be employed in the processes of the invention to reticulate elastomeric matrix 10. In another embodiment, all entrapped air in the pores of elastomeric matrix 10 is evacuated by application of vacuum prior to reticulation. In another embodiment, reticulation is accomplished through a plurality of reticulation steps. In another embodiment, two reticulation steps are used. In another embodiment, a first combustion reticulation is followed by a second combustion reticulation. In another embodiment, combustion reticulation is followed by chemical reticulation. In another embodiment, chemical reticulation is followed by combustion reticulation. In another embodiment, a first chemical reticulation is followed by a second chemical reticulation.

In one embodiment relating to orthopedic applications and the like, the elastomeric matrix 10 can be reticulated to provide an interconnected pore structure, the pores having an average diameter or other largest transverse dimension of at least about 10 μm. In another embodiment, the elastomeric matrix can be reticulated to provide pores with an average diameter or other largest transverse dimension of at least about 20 μm. In another embodiment, the elastomeric matrix can be reticulated to provide pores with an average diameter or other largest transverse dimension of at least about 50 μm. In another embodiment, the elastomeric matrix can be reticulated to provide pores with an average diameter or other largest transverse dimension of at least about 150 μm. In another embodiment, the elastomeric matrix can be reticulated to provide pores with an average diameter or other largest transverse dimension of at least about 250 μm. In another embodiment, the elastomeric matrix can be reticulated to provide pores with an average diameter or other largest transverse dimension of greater than about 250 μm. In another embodiment, the elastomeric matrix can be reticulated to provide pores with an average diameter or other largest transverse dimension of greater than 250 μm. In another embodiment, the elastomeric matrix can be reticulated to provide pores with an average diameter or other largest transverse dimension of at least about 450 μm. In another embodiment, the elastomeric matrix can be reticulated to provide pores with an average diameter or other largest transverse dimension of greater than about 450 μm. In another embodiment, the elastomeric matrix can be reticulated to provide pores with an average diameter or other largest transverse dimension of greater than 450 μm. In another embodiment, the elastomeric matrix can be reticulated to provide pores with an average diameter or other largest transverse dimension of at least about 500 μm.

In another embodiment relating to orthopedic applications and the like, the elastomeric matrix can be reticulated to provide pores with an average diameter or other largest transverse dimension of not greater than about 600 μm. In another embodiment, the elastomeric matrix can be reticulated to provide pores with an average diameter or other largest transverse dimension of not greater than about 450 μm. In another embodiment, the elastomeric matrix can be reticulated to provide pores with an average diameter or other largest transverse dimension of not greater than about 250 μm. In another embodiment, the elastomeric matrix can be reticulated to provide pores with an average diameter or other largest transverse dimension of not greater than about 150 μm. In another embodiment, the elastomeric matrix can be reticulated to provide pores with an average diameter or other largest transverse dimension of not greater than about 20 μm.

In another embodiment relating to orthopedic applications and the like, the elastomeric matrix can be reticulated to provide pores with an average diameter or other largest transverse dimension of from about 10 μm to about 50 μm. In another embodiment, the elastomeric matrix can be reticulated to provide pores with an average diameter or other largest transverse dimension of from about 20 μm to about 150 μm. In another embodiment, the elastomeric matrix can be reticulated to provide pores with an average diameter or other largest transverse dimension of from about 150 μm to about 250 μm. In another embodiment, the elastomeric matrix can be reticulated to provide pores with an average diameter or other largest transverse dimension of from about 250 μm to about 500 μm. In another embodiment, the elastomeric matrix can be reticulated to provide pores with an average diameter or other largest transverse dimension of from about 450 μm to about 600 μm. In another embodiment, the elastomeric matrix can be reticulated to provide pores with an average diameter or other largest transverse dimension of from about 10 μm to about 500 μm. In another embodiment, the elastomeric matrix can be reticulated to provide pores with an average diameter or other largest transverse dimension of from about 10 μm to about 600 μm.

Optionally, the reticulated elastomeric matrix may be purified, for example, by solvent extraction, either before or after reticulation. Any such solvent extraction, such as with isopropyl alcohol, or other purification process is, in one embodiment, a relatively mild process which is conducted so as to avoid or minimize possible adverse impact on the mechanical or physical properties of the elastomeric matrix that may be necessary to fulfill the objectives of this invention.

One embodiment employs chemical reticulation, where the elastomeric matrix is reticulated in an acid bath comprising an inorganic acid. Another embodiment employs chemical reticulation, where the elastomeric matrix is reticulated in a caustic bath comprising an inorganic base. Another embodiment employs solvent reticulation, where a volatile solvent that leaves no residue is used in the process. Another embodiment employs solvent reticulation at a temperature elevated above 25° C. In another embodiment, an elastomeric matrix comprising polycarbonate polyurethane is solvent reticulated with a solvent selected from tetrahydrofuran (“THF”), dimethyl acetamide (“DMAC”), dimethyl sulfoxide (“DMSO”), dimethylformamide (“DMF”), N-methyl-2-pyrrolidone, also known as m-pyrol, or a mixture thereof. In another embodiment, an elastomeric matrix comprising polycarbonate polyurethane is solvent reticulated with THF. In another embodiment, an elastomeric matrix comprising polycarbonate polyurethane is solvent reticulated with N-methyl-2-pyrrolidone. In another embodiment, an elastomeric matrix comprising polycarbonate polyurethane is chemically reticulated with a strong base. In another embodiment, the pH of the strong base is at least about 9.

In any of these chemical or solvent reticulation embodiments, the reticulated foam can optionally be washed. In any of these chemical or solvent reticulation embodiments, the reticulated foam can optionally be dried.

In one embodiment, combustion reticulation may be employed in which a combustible atmosphere, e.g., a mixture of hydrogen and oxygen or methane and oxygen, is ignited, e.g., by a spark. In another embodiment, combustion reticulation is conducted in a pressure chamber. In another embodiment, the pressure in the pressure chamber is substantially reduced, e.g., to below about 50-150 millitorr by evacuation for at least about 2 minutes, before, e.g., hydrogen, oxygen or a mixture thereof, is introduced. In another embodiment, the pressure in the pressure chamber is substantially reduced in more than one cycle, e.g., the pressure is substantially reduced, an unreactive gas such as argon or nitrogen is introduced then the pressure is again substantially reduced, before hydrogen, oxygen or a mixture thereof is introduced. The temperature at which reticulation occurs can be influenced by, e.g., the temperature at which the chamber is maintained and/or by the hydrogen/oxygen ratio in the chamber. In another embodiment, combustion reticulation is followed by an annealing period. In any of these combustion reticulation embodiments, the reticulated foam can optionally be washed. In any of these combustion reticulation embodiments, the reticulated foam can optionally be dried.

In one embodiment, the reticulated elastomeric matrix's permeability to a fluid, e.g., a liquid, is greater than the permeability to the fluid of an unreticulated matrix from which the reticulated elastomeric matrix was made. In another embodiment, the reticulation process is conducted to provide an elastomeric matrix configuration favoring cellular ingrowth and proliferation into the interior of the matrix. In another embodiment, the reticulation process is conducted to provide an elastomeric matrix configuration which favors cellular ingrowth and proliferation throughout the elastomeric matrix configured for implantation, as described herein.

The term “configure” and the like is used to denote the arranging, shaping and dimensioning of the respective structure to which the term is applied. Thus, reference to a structure as being “configured” for a purpose is intended to reference the whole spatial geometry of the relevant structure or part of a structure as being selected or designed to serve the stated purpose.

Imparting Endopore Features

Within pores 20, elastomeric matrix 10 may, optionally, have features in addition to the void or gas-filled volume described above. In one embodiment, elastomeric matrix 10 may have what are referred to herein as “endopore” features as part of its microstructure, i.e., features of elastomeric matrix 10 that are located “within the pores”. In one embodiment, the internal surfaces of pores 20 may be “endoporously coated”, i.e., coated or treated to impart to those surfaces a degree of a desired characteristic, e.g., hydrophilicity. The coating or treating medium can have additional capacity to transport or bond to active ingredients that can then be preferentially delivered to pores 20. In one embodiment, this coating medium or treatment can be used facilitate covalent bonding of materials to the interior pore surfaces, for example, as are described in the applications to which priority is claimed. In another embodiment, the coating comprises a biodegradable or absorbable polymer and an inorganic component, such as hydroxyapatite. Hydrophilic treatments may be effected by chemical or radiation treatments on the fabricated reticulated elastomeric matrix 10, by exposing the elastomer to a hydrophilic, e.g., aqueous, environment during elastomer setting, or by other means known to those skilled in the art.

Furthermore, one or more coatings may be applied endoporously by contacting with a film-forming biocompatible polymer either in a liquid coating solution or in a melt state under conditions suitable to allow the formation of a biocompatible polymer film. In one embodiment, the polymers used for such coatings are film-forming biocompatible polymers with sufficiently high molecular weight so as not to be waxy or tacky. The polymers should also adhere to the solid phase 12. In another embodiment, the bonding strength is such that the polymer film does not crack or dislodge during handling or deployment of reticulated elastomeric matrix 10.

Suitable biocompatible polymers include polyamides, polyolefins (e.g., polypropylene, polyethylene), nonabsorbable polyesters (e.g., polyethylene terephthalate), and bioabsorbable aliphatic polyesters (e.g., homopolymers and copolymers of lactic acid, glycolic acid, lactide, glycolide, para-dioxanone, trimethylene carbonate, ∈-caprolactone or a mixture thereof). Further, biocompatible polymers include film-forming bioabsorbable polymers; these include aliphatic polyesters, poly(amino acids), copoly(ether-esters), polyalkylenes oxalates, polyamides, poly(iminocarbonates), polyorthoesters, polyoxaesters including polyoxaesters containing amido groups, polyamidoesters, polyanhydrides, polyphosphazenes, biomolecules or a mixture thereof. For the purpose of this invention aliphatic polyesters include polymers and copolymers of lactide (which includes lactic acid d-, l- and meso lactide), ∈-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, 6,6-dimethyl-1,4-dioxan-2-one or a mixture thereof. In one embodiment, the reinforcement can be made from biopolymer, such as collagen, elastin, and the like. The biopolymer can be biodegradable or bioabsorbable.

Biocompatible polymers further include film-forming biodurable polymers with relatively low chronic tissue response, such as polyurethanes, silicones, poly(meth)acrylates, polyesters, polyalkyl oxides (e.g., polyethylene oxide), polyvinyl alcohols, polyethylene glycols and polyvinyl pyrrolidone, as well as hydrogels, such as those formed from cross-linked polyvinyl pyrrolidinone and polyesters. Other polymers can also be used as the biocompatible polymer provided that they can be dissolved, cured or polymerized. Such polymers and copolymers include polyolefins, polyisobutylene and ethylene-α-olefin copolymers; acrylic polymers (including methacrylates) and copolymers; vinyl halide polymers and copolymers, such as polyvinyl chloride; polyvinyl ethers, such as polyvinyl methyl ether; polyvinylidene halides such as polyvinylidene fluoride and polyvinylidene chloride; polyacrylonitrile; polyvinyl ketones; polyvinyl aromatics such as polystyrene; polyvinyl esters such as polyvinyl acetate; copolymers of vinyl monomers with each other and with α-olefins, such as etheylene-methyl methacrylate copolymers and ethylene-vinyl acetate copolymers; acrylonitrile-styrene copolymers; ABS resins; polyamides, such as nylon 66 and polycaprolactam; alkyd resins; polycarbonates; polyoxymethylenes; polyimides; polyethers; epoxy resins; polyurethanes; rayon; rayon-triacetate; cellophane; cellulose and its derivatives such as cellulose acetate, cellulose acetate butyrate, cellulose nitrate, cellulose propionate and cellulose ethers (e.g., carboxymethyl cellulose and hydroxyalkyl celluloses); or a mixture thereof. For the purpose of this invention, polyamides include polyamides of the general forms:

—N(H)—(CH₂)ₙ—C(O)— and —N(H)—(CH₂)ₙ—N(H)—C(O)—(CH₂)_{y}—C(O)—,

where n is an integer from about 4 to about 13; x is an integer from about 4 to about 12; and y is an integer from about 4 to about 16. It is to be understood that the listings of materials above are illustrative but not limiting.

A device made from reticulated elastomeric matrix 10 generally is coated by simple dip or spray coating with a polymer, optionally comprising a pharmaceutically-active agent, such as a therapeutic agent or drug. In one embodiment, the coating is a solution and the polymer content in the coating solution is from about 1% to about 40% by weight. In another embodiment, the polymer content in the coating solution is from about 1% to about 20% by weight. In another embodiment, the polymer content in the coating solution is from about 1% to about 10% by weight.

The solvent or solvent blend for the coating solution is chosen with consideration given to, inter alia, the proper balancing of viscosity, deposition level of the polymer, wetting rate and evaporation rate of the solvent to properly coat solid phase 12, as known to those in the art. In one embodiment, the solvent is chosen such the polymer is soluble in the solvent. In another embodiment, the solvent is substantially completely removed from the coating. In another embodiment, the solvent is non-toxic, non-carcinogenic and environmentally benign. Mixed solvent systems can be advantageous for controlling the viscosity and evaporation rates. In all cases, the solvent should not react with the coating polymer. Solvents include by are not limited to: acetone, N-methylpyrrolidone (“NMP”), DMSO, toluene, methylene chloride, chloroform, 1,1,2-trichloroethane (“TCE”), various freons, dioxane, ethyl acetate, THF, DMF and DMAC.

In another embodiment, the film-forming coating polymer is a thermoplastic polymer that is melted, enters the pores 20 of the elastomeric matrix 10 and, upon cooling or solidifying, forms a coating on at least a portion of the solid material 12 of the elastomeric matrix 10. In another embodiment, the processing temperature of the thermoplastic coating polymer in its melted form is above about 60° C. In another embodiment, the processing temperature of the thermoplastic coating polymer in its melted form is above about 90° C. In another embodiment, the processing temperature of the thermoplastic coating polymer in its melted form is above about 120° C.

In a further embodiment of the invention, described in more detail below, some or all of the pores 20 of elastomeric matrix 10 are coated or filled with a cellular ingrowth promoter. In another embodiment, the promoter can be foamed. In another embodiment, the promoter can be present as a film. The promoter can be a biodegradable or absorbable material to promote cellular invasion of elastomeric matrix 10 in vivo. Promoters include naturally occurring materials that can be enzymatically degraded in the human body or are hydrolytically unstable in the human body, such as fibrin, fibrinogen, collagen, elastin, hyaluronic acid and absorbable biocompatible polysaccharides, such as chitosan, starch, fatty acids (and esters thereof), glucoso-glycans and hyaluronic acid. In some embodiments, the pore surface of elastomeric matrix 10 is coated or impregnated, as described in the previous section but substituting the promoter for the biocompatible polymer or adding the promoter to the biocompatible polymer, to encourage cellular ingrowth and proliferation.

In one embodiment, the coating or impregnating process is conducted so as to ensure that the product “composite elastomeric implantable device”, i.e., a reticulated elastomeric matrix and a coating, as used herein, retains sufficient resiliency after compression such that it can be delivery-device delivered, e.g., catheter, syringe or endoscope delivered. Some embodiments of such a composite elastomeric implantable device will now be described with reference to collagen, by way of non-limiting example, with the understanding that other materials may be employed in place of collagen, as described above.

One embodiment of the invention is a process for preparing a composite elastomeric implantable device comprising:

a) infiltrating an aqueous collagen slurry into the pores of a reticulated, porous elastomer, such as elastomeric matrix 10, which is optionally a biodurable elastomer product; and

b) removing the water, optionally by lyophilizing, to provide a collagen coating, where the collagen coating optionally comprises an interconnected network of pores, on at least a portion of a pore surface of the reticulated, porous elastomer.

Collagen may be infiltrated by forcing, e.g., with pressure, an aqueous collagen slurry, suspension or solution into the pores of an elastomeric matrix. The collagen may be Type I, II or III or a mixture thereof. In one embodiment, the collagen type comprises at least 90% collagen I. The concentration of collagen is from about 0.3% to about 2.0% by weight and the pH of the slurry, suspension or solution is adjusted to be from about 2.6 to about 5.0 at the time of lyophilization. Alternatively, collagen may be infiltrated by dipping an elastomeric matrix into a collagen slurry.

As compared with the uncoated reticulated elastomer, the composite elastomeric implantable device can have a void phase 14 that is slightly reduced in volume. In one embodiment, the composite elastomeric implantable device retains good fluid permeability and sufficient porosity for ingrowth and proliferation of fibroblasts or other cells.

Optionally, the lyophilized collagen can be cross-linked to control the rate of in vivo enzymatic degradation of the collagen coating and/or to control the ability of the collagen coating to bond to elastomeric matrix 10. The collagen can be cross-linked by methods known to those in the art, e.g., by heating in an evacuated chamber, by heating in a substantially moisture-free inert gas atmosphere, by bring the collagen into contact with formaldehyde vapor, or by the use of glutaraldehyde. Without being bound by any particular theory, it is thought that when the composite elastomeric implantable device is implanted, tissue-forming agents that have a high affinity to collagen, such as fibroblasts, will more readily invade the collagen-impregnated elastomeric matrix 10 than the uncoated matrix. It is further thought, again without being bound by any particular theory, that as the collagen enzymatically degrades, new tissue invades and fills voids left by the degrading collagen while also infiltrating and filling other available spaces in the elastomeric matrix 10. Such a collagen coated or impregnated elastomeric matrix 10 is thought, without being bound by any particular theory, to be additionally advantageous for the structural integrity provided by the reinforcing effect of the collagen within the pores 20 of the elastomeric matrix 10, which can impart greater rigidity and structural stability to various configurations of elastomeric matrix 10.

Processes of preparing a collagen-coated composite elastomeric implantable device is exemplified in Examples 3 and 12. Other processes will be apparent to those skilled in the art.

Coated Implantable Devices

In some applications, a device made from elastomeric matrix 10 can have at least a portion of the outermost or macro surface coated or fused in order to present a smaller macro surface area, because the internal surface area of pores below the surface is no longer accessible. Without being bound by any particular theory, it is thought that this decreased surface area provides more predictable and easier delivery and transport through long tortuous channels inside delivery-devices. Surface coating or fusion alters the “porosity of the surface”, i.e., at least partially reduces the percentage of pores open to the surface, or, in the limit, completely closes-off the pores of a coated or fused surface, i.e., that surface is nonporous because it has substantially no pores remaining on the coated or fused surface. However, surface coating or fusion still allows the internal interconnected porous structure of elastomeric matrix 10 to remain open internally and on other non-coated or non-fused surfaces; e.g., the portion of a coated or fused pore not at the surface remains interconnected to other pores, and those remaining open surfaces can foster cellular ingrowth and proliferation. In one embodiment, a coated and uncoated surface are orthogonal to each other. In another embodiment, a coated and uncoated surface are at an oblique angle to each other. In another embodiment, a coated and uncoated surface are adjacent. In another embodiment, a coated and uncoated surface are nonadjacent. In another embodiment, a coated and uncoated surface are in contact with each other. In another embodiment, a coated and uncoated surface are not in contact with each other.

In other applications, one or more planes of the macro surface of an implantable device made from reticulated elastomeric matrix 10 may be coated, fused or melted to improve its attachment efficiency to attaching means, e.g., anchors or sutures, so that the attaching means does not tear-through or pull-out from the implantable device. Without being bound by any particular theory, creation of additional contact anchoring macro surface(s) on the implantable device, as described above, is thought to inhibit tear-through or pull-out by providing fewer voids and greater resistance.

The fusion and/or selective melting of the macro surface layer of elastomeric matrix 10 can be brought about in several different ways. In one embodiment, a knife or a blade used to cut a block of elastomeric matrix 10 into sizes and shapes for making final implantable devices can be heated to an elevated temperature, for example, as exemplified in Example 9. In another embodiment, a device of desired shape and size is cut from a larger block of elastomeric matrix 10 by using a laser cutting device and, in the process, the surfaces that come into contact with the laser beam are fused. In another embodiment, a cold laser cutting device is used to cut a device of desired shape and size. In yet another embodiment, a heated mold can be used to impart the desired size and shape to the device by the process of heat compression. A slightly oversized elastomeric matrix 10, cut from a larger block, can be placed into a heated mold. The mold is closed over the cut piece to reduce its overall dimensions to the desired size and shape and fuse those surfaces in contact with the heated mold, for example, as exemplified in Example 10. In each of the aforementioned embodiments, the processing temperature for shaping and sizing is greater than about 15° C. in one embodiment. In another embodiment, the processing temperature for shaping and sizing is in excess of about 100° C. In another embodiment, the processing temperature for shaping and sizing is in excess of about 130° C. In another embodiment, the layer(s) and/or portions of the macro surface not being fused are protected from exposure by covering them during the fusing of the macro surface.

The coating on the macro surface can be made from a biocompatible polymer, which can include be both biodegradable or absorbable and non-biodegradable or non-absorbable polymers. Suitable absorbable polymers include those biocompatible polymers disclosed in the previous section. It is to be understood that that listing of materials is illustrative but not limiting. In one embodiment, surface pores are closed by applying an absorbable polymer melt coating onto a shaped elastomeric matrix. Together, the elastomeric matrix and the coating form the device. In another embodiment, surface pores are closed by applying an absorbable polymer solution coating onto a shaped elastomeric matrix to form a device. In another embodiment, the coating and the elastomeric matrix, taken together, occupy a larger volume than the uncoated elastomeric matrix alone.

The coating on elastomeric matrix 10 can be applied by, e.g., dipping or spraying a coating solution comprising a polymer or a polymer that is admixed with a pharmaceutically-active agent. In one embodiment, the polymer content in the coating solution is from about 1% to about 40% by weight. In another embodiment, the polymer content in the coating solution is from about 1% to about 20% by weight. In another embodiment, the polymer content in the coating solution is from about 1% to about 10% by weight. In another embodiment, the layer(s) and/or portions of the macro surface not being solution-coated are protected from exposure by covering them during the solution-coating of the macro surface. The solvent or solvent blend for the coating solution is chosen, e.g., based on the considerations discussed in the previous section (i.e., in the “Imparting Endopore Features” section).

In one embodiment, the coating on elastomeric matrix 10 may be applied by melting a film-forming coating polymer and applying the melted polymer onto the elastomeric matrix 10 by dip coating, for example, as exemplified in Example 11. In another embodiment, the coating on elastomeric matrix 10 may be applied by melting the film-forming coating polymer and applying the melted polymer through a die, in a process such as extrusion or coextrusion, as a thin layer of melted polymer onto a mandrel formed by elastomeric matrix 10. In either of these embodiments, the melted polymer coats the macro surface and bridges or plugs pores of that surface but does not penetrate into the interior to any significant depth. Without being bound by any particular theory, this is thought to be due to the high viscosity of the melted polymer. Thus, the reticulated nature of portions of the elastomeric matrix removed from the macro surface, and portions of the elastomeric matrix's macro surface not in contact with the melted polymer, is maintained. Upon cooling and solidifying, the melted polymer forms a layer of solid coating on the elastomeric matrix 10. In one embodiment, the processing temperature of the melted thermoplastic coating polymer is at least about 60° C. In another embodiment, the processing temperature of the melted thermoplastic coating polymer is at least above about 90° C. In another embodiment, the processing temperature of the melted thermoplastic coating polymer is at least above about 120° C. In another embodiment, the layer(s) and/or portions of the macro surface not being melt-coated are protected from exposure by covering them during the melt-coating of the macro surface.

Another embodiment of the invention employs a collagen-coated composite elastomeric implantable device, as described above, configured as a sleeve extending around the implantable device. The collagen matrix sleeve can be implanted at a tissue repair and regeneration site, either adjacent to and in contact with that site. So located, the collagen matrix sleeve can be useful to help retain the elastomeric matrix 10, facilitate the formation of a tissue seal and help prevent leakage. The presence of the collagen in elastomeric matrix 10 can enhance cellular ingrowth and proliferation and improve mechanical stability, in one embodiment, by enhancing the attachment of fibroblasts to the collagen. The presence of collagen can stimulate earlier and/or more complete infiltration of the interconnected pores of elastomeric matrix 10.

Tissue Culture

The biodurable reticulated elastomeric matrix of this invention can support cell types including cells secreting structural proteins and cells that produce proteins characterizing organ function. The ability of the elastomeric matrix to facilitate the co-existence of multiple cell types together and its ability to support protein secreting cells demonstrates the applicability of the elastomeric matrix in organ growth in vitro or in vivo and in organ reconstruction. In addition, the biodurable reticulated elastomeric matrix may also be used in the scale up of human cell lines for implantation to the body for many applications including implantation of fibroblasts, chondrocytes, osteoblasts, osteoclasts, osteocytes, synovial cells, bone marrow stromal cells, stem cells, fibrocartilage cells, endothelial cells, smooth muscle cells, adipocytes, cardiomyocytes, myocytes, keratinocytes, hepatocytes, leukocytes, macrophages, endocrine cells, genitourinary cells, lymphatic vessel cells, pancreatic islet cells, muscle cells, intestinal cells, kidney cells, blood vessel cells, thyroid cells, parathyroid cells, cells of the adrenal-hypothalamic pituitary axis, bile duct cells, ovarian or testicular cells, salivary secretory cells, renal cells, epithelial cells, nerve cells, stem cells, progenitor cells, myoblasts and intestinal cells.

The approach to engineer new tissue can be obtained through implantation of cells seeded in elastomeric matrices (either prior to or concurrent to or subsequent to implantation). In this case, the elastomeric matrices may be configured either in a closed manner to protect the implanted cells from the body's immune system, or in an open manner so that the new cells can be incorporated into the body. Thus in another embodiment, the cells may be incorporated, i.e. cultured and proliferated, onto the elastomeric matrix prior, concurrent or subsequent to implantation of the elastomeric matrix in the patient.

In one embodiment, the implantable device made from biodurable reticulated elastomeric matrix can be seeded with a type of cell and cultured before being inserted into the patient, optionally using a delivery-device, for the explicit purpose of tissue repair or tissue regeneration. It is necessary to perform the tissue or cell culture in a suitable culture medium with or without stimulus such as stress or orientation. The cells include fibroblasts, chondrocytes, osteoblasts, osteoclasts, osteocytes, synovial cells, bone marrow stromal cells, stem cells, fibrocartilage cells, endothelial cells and smooth muscle cells.

Surfaces on the biodurable reticulated elastomeric matrix possessing different pore morphology, size, shape and orientation may be cultured with different type of cells to develop cellular tissue engineering implantable devices that are specifically targeted towards orthopedic applications, especially in soft tissue attachment, repair, regeneration, augmentation and/or support encompassing the spine, shoulder, knee, hand or joints, and in the growth of a prosthetic organ. In another embodiment, all the surfaces on the biodurable reticulated elastomeric matrix possessing similar pore morphology, size, shape and orientation may be so cultured.

In other embodiments, the biodurable reticulated elastomeric matrix of this invention may have applications in the areas of mammary prostheses, pacemaker housings, LVAD bladders or as a tissue bridging matrix.

Pharmaceutically-Active Agent Delivery

In another embodiment, the film-forming polymer used to coat reticulated elastomeric matrix 10 can provide a vehicle for the delivery of and/or the controlled release of a pharmaceutically-active agent, for example, a drug, such as is described in the applications to which priority is claimed. In another embodiment, the pharmaceutically-active agent is admixed with, covalently bonded to, adsorbed onto and/or absorbed into the coating of elastomeric matrix 10 to provide a pharmaceutical composition. In another embodiment, the components, polymers and/or blends used to form the foam comprise a pharmaceutically-active agent. To form these foams, the previously described components, polymers and/or blends are admixed with the pharmaceutically-active agent prior to forming the foam or the pharmaceutically-active agent is loaded into the foam after it is formed.

In one embodiment, the coating polymer and pharmaceutically-active agent have a common solvent. This can provide a coating that is a solution. In another embodiment, the pharmaceutically-active agent can be present as a solid dispersion in a solution of the coating polymer in a solvent.

A reticulated elastomeric matrix 10 comprising a pharmaceutically-active agent may be formulated by mixing one or more pharmaceutically-active agent with the polymer used to make the foam, with the solvent or with the polymer-solvent mixture and foamed. Alternatively, a pharmaceutically-active agent can be coated onto the foam, in one embodiment, using a pharmaceutically-acceptable carrier. If melt-coating is employed, then, in another embodiment, the pharmaceutically-active agent withstands melt processing temperatures without substantial diminution of its efficacy.

Formulations comprising a pharmaceutically-active agent can be prepared from one or more pharmaceutically-active agents by admixing, covalently bonding, adsorbing onto and/or absorbing into the same with the coating of the reticulated elastomeric matrix 10 or by incorporating the pharmaceutically-active agent into additional hydrophobic or hydrophilic coatings. The pharmaceutically-active agent may be present as a liquid, a finely divided solid or another appropriate physical form. Typically, but optionally, the matrix can include one or more conventional additives, such as diluents, carriers, excipients, stabilizers and the like.

In another embodiment, a top coating can be applied to delay release of the pharmaceutically-active agent. In another embodiment, a top coating can be used as the matrix for the delivery of a second pharmaceutically-active agent. A layered coating, comprising respective layers of fast- and slow-hydrolyzing polymer, can be used to stage release of the pharmaceutically-active agent or to control release of different pharmaceutically-active agents placed in the different layers. Polymer blends may also be used to control the release rate of different pharmaceutically-active agents or to provide a desirable balance of coating characteristics (e.g., elasticity, toughness) and drug delivery characteristics (e.g., release profile). Polymers with differing solvent solubilities can be used to build-up different polymer layers that may be used to deliver different pharmaceutically-active agents or to control the release profile of a pharmaceutically-active agents.

The amount of pharmaceutically-active agent present depends upon the particular pharmaceutically-active agent employed and medical condition being treated. In one embodiment, the pharmaceutically-active agent is present in an effective amount. In another embodiment, the amount of pharmaceutically-active agent represents from about 0.01% to about 60% of the coating by weight. In another embodiment, the amount of pharmaceutically-active agent represents from about 0.01% to about 40% of the coating by weight. In another embodiment, the amount of pharmaceutically-active agent represents from about 0.1% to about 20% of the coating by weight.

Many different pharmaceutically-active agents can be used in conjunction with the reticulated elastomeric matrix. In general, pharmaceutically-active agents that may be administered via pharmaceutical compositions of this invention include, without limitation, any therapeutic or pharmaceutically-active agent (including but not limited to nucleic acids, proteins, lipids, and carbohydrates) that possesses desirable physiologic characteristics for application to the implant site or administration via a pharmaceutical compositions of the invention. Therapeutics include, without limitation, antiinfectives such as antibiotics and antiviral agents; chemotherapeutic agents (e.g., anticancer agents); anti-rejection agents; analgesics and analgesic combinations; anti-inflammatory agents; hormones such as steroids; growth factors (including but not limited to cytokines, chemokines, and interleukins) and other naturally derived or genetically engineered proteins, polysaccharides, glycoproteins and lipoproteins. These growth factors are described in The Cellular and Molecular Basis of Bone Formation and Repair by Vicki Rosen and R. Scott Thies, published by R. G. Landes Company, hereby incorporated herein by reference. Additional therapeutics include thrombin inhibitors, antithrombogenic agents, thrombolytic agents, fibrinolytic agents, vasospasm inhibitors, calcium channel blockers, vasodilators, antihypertensive agents, antimicrobial agents, antibiotics, inhibitors of surface glycoprotein receptors, antiplatelet agents, antimitotics, microtubule inhibitors, anti secretory agents, actin inhibitors, remodeling inhibitors, antisense nucleotides, anti metabolites, antiproliferatives, anticancer chemotherapeutic agents, anti-inflammatory steroids, non-steroidal anti-inflammatory agents, immunosuppressive agents, growth hormone antagonists, growth factors, dopamine agonists, radiotherapeutic agents, peptides, proteins, enzymes, extracellular matrix components, angiotensin-converting enzyme (ACE) inhibitors, free radical scavengers, chelators, antioxidants, anti polymerases, antiviral agents, photodynamic therapy agents and gene therapy agents.

Additionally, various proteins (including short chain peptides), growth agents, chemotatic agents, growth factor receptors or ceramic particles can be added to the foams during processing, adsorbed onto the surface or back-filled into the foams after the foams are made. For example, in one embodiment, the pores of the foam may be partially or completely filled with biocompatible resorbable synthetic polymers or biopolymers (such as collagen or elastin), biocompatible ceramic materials (such as hydroxyapatite), and combinations thereof, and may optionally contain materials that promote tissue growth through the device. Such tissue-growth materials include but are not limited to autograft, allograft or xenograft bone, bone marrow and morphogenic proteins. Biopolymers can also be used as conductive or chemotactic materials, or as delivery vehicles for growth factors. Examples include recombinant collagen, animal-derived collagen, elastin and hyaluronic acid. Pharmaceutically-active coatings or surface treatments could also be present on the surface of the materials. For example, bioactive peptide sequences (RGD's) could be attached to the surface to facilitate protein adsorption and subsequent cell tissue attachment.

Bioactive molecules include, without limitation, proteins, collagens (including types IV and XVIII), fibrillar collagens (including types I, II, III, V, XI), FACIT collagens (types IX, XII, XIV), other collagens (types VI, VII, XIII), short chain collagens (types VIII, X), elastin, entactin-1, fibrillin, fibronectin, fibrin, fibrinogen, fibroglycan, fibromodulin, fibulin, glypican, vitronectin, laminin, nidogen, matrilin, perlecan, heparin, heparan sulfate proteoglycans, decorin, filaggrin, keratin, syndecan, agrin, integrins, aggrecan, biglycan, bone sialoprotein, cartilage matrix protein, Cat-301 proteoglycan, CD44, cholinesterase, HB-GAM, hyaluronan, hyaluronan binding proteins, mucins, osteopontin, plasminogen, plasminogen activator inhibitors, restrictin, serglycin, tenascin, thrombospondin, tissue-type plasminogen activator, urokinase type plasminogen activator, versican, von Willebrand factor, dextran, arabinogalactan, chitosan, polyactide-glycolide, alginates, pullulan, gelatin and albumin.

Additional bioactive molecules include, without limitation, cell adhesion molecules and matricellular proteins, including those of the immunoglobulin (Ig; including monoclonal and polyclonal antibodies), cadherin, integrin, selectin, and H-CAM superfamilies. Examples include, without limitation, AMOG, CD2, CD4, CD8, C-CAM (CELL-CAM 105), cell surface galactosyltransferase, connexins, desmocollins, desmoglein, fasciclins, F11, GP Ib-IX complex, intercellular adhesion molecules, leukocyte common antigen protein tyrosine phosphate (LCA, CD45), LFA-1, LFA-3, mannose binding proteins (MBP), MTJC18, myelin associated glycoprotein (MAG), neural cell adhesion molecule (NCAM), neurofascin, neruoglian, neurotactin, netrin, PECAM-1, PH-20, semaphorin, TAG-1, VCAM-1, SPARC/osteonectin, CCN1 (CYR61), CCN2 (CTGF; Connective Tissue Growth Factor), CCN3 (NOV), CCN4 (WISP-1), CCN5 (WISP-2), CCN6 (WISP-3), occludin and claudin. Growth factors include, without limitation, BMP's (1-7), BMP-like Proteins (GFD-5, -7, -8), epidermal growth factor (EGF), erythropoietin (EPO), fibroblast growth factor (FGF), growth hormone (GH), growth hormone releasing factor (GHRF), granulocyte colony-stimulating factor (G-CSF), granulocyte-macrophage colony-stimulating factor (GM-CSF), insulin, insulin-like growth factors (IGF-I, IGF-II), insulin-like growth factor binding proteins (IGFBP), macrophage colony-stimulating factor (M-CSF), Multi-CSF (II-3), platelet-derived growth factor (PDGF), tumor growth factors (TGF-alpha, TGF-beta), tumor necrosis factor (TNF-alpha), vascular endothelial growth factors (VEGF's), angiopoietins, placenta growth factor (PIGF), interleukins, and receptor proteins or other molecules that are known to bind with the aforementioned factors. Short-chain peptides include, without limitation (designated by single letter amino acid code), RGD, EILDV, RGDS, RGES, RFDS, GRDGS, GRGS, GRGDTP and QPPRARI.

Compressive Molding

In addition to varying elastomeric matrix 10's chemistry and/or processing in order to obtain a range of desirable or targeted implantable device performance, post-reticulation steps, such as imparting endpore features (already discussed above) can also be used to obtain a range of desirable or targeted implantable device performance. In another post-reticulation embodiment, the reticulated elastomeric matrix is compressed in at least one dimension, e.g., 1-dimensional compression, 2-dimensional compression, or 3-dimensional compression, in a compressive molding process and, if reinforced with a reinforcement as discussed in detail below, remains compressed during the inclusion of the reinforcement.

In one embodiment, the implantable device is made from a reticulated elastomeric matrix such that the device's density is from about 2.0 lbs/ft³ to about 4.0 lbs/ft³ (from about 0.032 g/cc to about 0.064 g/cc). In another embodiment, the implantable device is made such that the device's density is from about 4.0 lbs/ft³ to about 8.0 lbs/ft³ (from about 0.064 g/cc to about 0.128 g/cc). In another embodiment, the implantable device is made such that the device's density is from about 2.5 lbs/ft³ to about 26 lbs/ft³ (from about 0.040 g/cc to about 0.417 g/cc).

In one embodiment, the implantable device is made from a matrix that is oriented in one dimension. In another embodiment, the implantable device is made from a matrix that is oriented in two dimensions. In another embodiment, the implantable device is made from a matrix that is oriented in three dimensions. In another embodiment, there is substantially no preferred orientation in the matrix. In another embodiment, the matrix orientation occurs during initial foam formation. In another embodiment, the matrix orientation occurs during reticulation. In another embodiment, the matrix orientation occurs during any secondary processing, such as by compressive molding, that may occur subsequent to reticulation. The results of orientation are manifested by enhanced properties and/or enhanced performance in the direction of orientation. For example, tensile properties, such as tensile strength, can be enhanced in the foam rise direction while only a slight change or no significant change in tensile strength occurs in the directions orthogonal to the foam rise direction.

In one secondary processing method, referred to herein as compressive molding, desirable enhanced performance is obtained by densification and/or orientation in one dimension, two dimensions or three dimensions using different temperatures. In one embodiment, the densification and/or orientation can be effected without the use of a mold. In another embodiment, the densification and/or orientation is facilitated by using a mold. As discussed below, the densification and/or orientation is usually carried out at a temperature above 25° C., e.g., from about 105° C. to about 180° C., over a period of time where the length of time depends on the temperature(s) used. In another embodiment, the compressive molding process is conducted in a batch process. In another embodiment, the compressive molding process is conducted in a continuous process.

A “preform” is a shaped uncompressed reticulated elastomeric matrix that has been cut or machined from a block of reticulated elastomeric matrix for use in secondary processing, such as compressive molding. The preform can have a predetermined size and shape. In one embodiment, the size and shape of the preform is determined by the final or desired compression ratio that will be imparted during compressive molding.

When a mold is used, the mold cavity can have fixed shape, such as a cylinder, cube, sphere or ellipsoid, or it can have an irregular shape. The reticulated cross-linked biodurable elastomeric polycarbonate urea-urethane matrix, upon being compressive molded, conforms to a great degree to the geometry of the mold at the end of the densification and/or orientation step.

Compressive molding can also be carried out in a molds who's contours can change during the compressive molding process, e.g., from an initial shape and/or size to a final shape and/or size. The change in the dimension of this mold can be initiated or activated by application of heat or application of load. In one such example, a cylindrically-shaped preform of reticulated elastomeric matrix having diameter d3 was placed inside a thin-walled PTFE (poly(tetrafluoroethylene)) shrink-wrap tube having initial diameter, d1, greater than d3. Upon application of external heat and/or load, the PTFE shrink-wrap tube shrunk from its initial diameter d1 to a smaller final diameter of d2. The cylindrical preform with diameter d3 was compressed to a final diameter substantially equal to or equal to d2. The compressed reticulated elastomeric matrix conformed to a great degree to the geometry of the mold which, in this embodiment, was the heat-shrunk PTFE tubing.

In one embodiment, the densification and/or orientation believed to be imparted to the reticulated elastomeric matrix by compressive molding results in property enhancement and/or performance enhancement for the compressed reticulated elastomeric matrix, such as in its mechanical properties, e.g., tensile strength, tensile modulus, compressive strength, compressive, modulus and/or tear strength. In another embodiment, the densification and/or orientation believed to be imparted to the reticulated elastomeric matrix by compressive molding results in performance enhancement related to delivery, conformability, handling and/or filling at the tissue healing site.

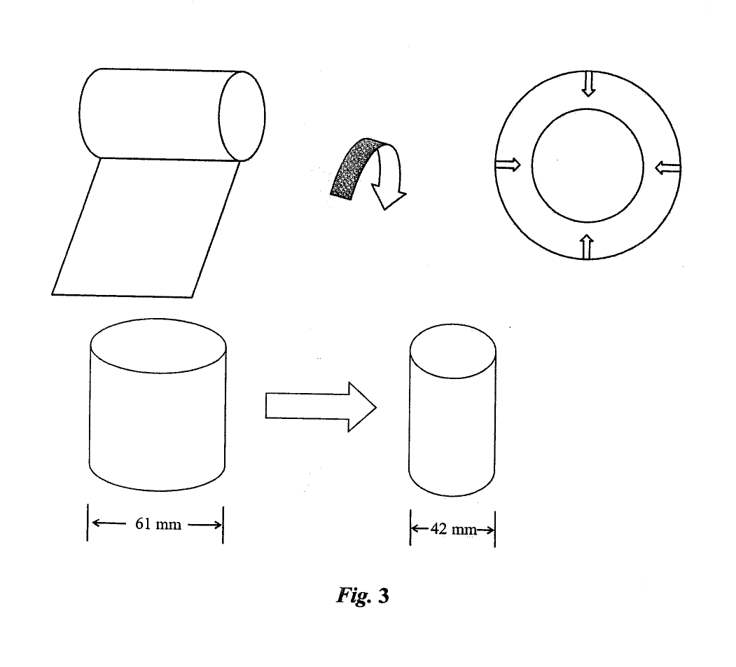

During compressive molding, in one embodiment at least one dimension of the preform, e.g., the length and/or diameter of a cylindrical preform, is reduced in size. A non-limiting compressive molding process for reducing the diameter of a cylindrical preform with substantially no change in its length through the use of a mold is illustrated in FIG. 3. An exemplary cylindrical preform, 61 mm in diameter in FIG. 3, can be placed inside a mold formed from a cylindrically-shaped flexible sheet, e.g., a thin aluminum, steel or plastic sheet. One edge of the sheet is secured in any appropriate way while the other end, the tail, protrudes. Then, force can be applied to pull the tail away from the cylindrical portion of the sheet thereby reducing the inside diameter of the sheet and, concurrently, reducing the diameter of the preform held within the sheet, as illustrated in FIG. 3. The exemplary 61 mm diameter cylindrical preform of FIG. 3 can be reduced to, e.g., 42 mm, as illustrated therein. During this compressive molding process, the inner mold surface is believed to move or be displaced relative to the outside surface of the preform in contact with the inner mold surface before the tail is pulled; therefore, this process of compressive molding can also be described as a “moving mold wall” compressive molding process.

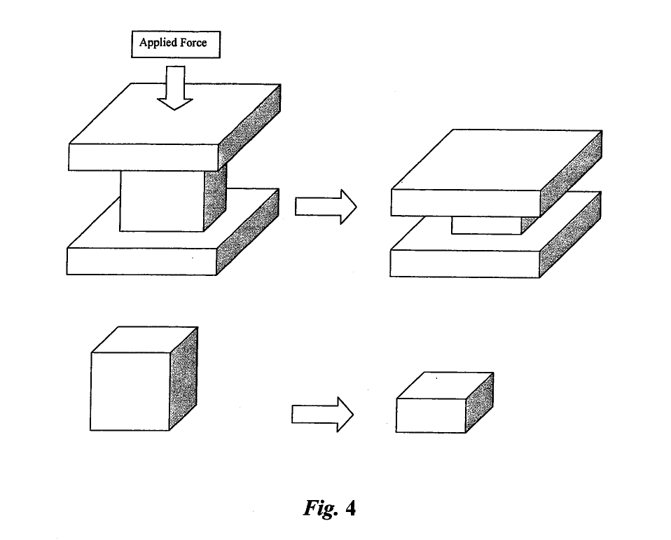

In another embodiment, during compressive molding one dimension of a preform, such as the thickness dimension of a cube, is reduced while its other two dimensions remain substantially unchanged. This is illustrated in FIG. 4. An exemplary cubical preform can be placed inside a mold formed from two opposed relatively rigid mold faces of, e.g., thick aluminum, steel or plastic. Then, force can be applied to push the faces closer together, thereby reducing the thickness dimension of the cube held between the faces, as illustrated in FIG. 4. During this compressive molding process, each face is believed to be approximately motionless or fixed relative to the outside surface of the preform in contact with a face as they are pushed closer together; therefore, this process of compressive molding can also be described as a “fixed mold wall” compressive molding process.

In another embodiment, substantially all of the changes in preform volume occurring upon compressive molding can be accounted for by the dimensional change occurring only in one dimension. In another embodiment, all of the changes in preform volume occurring upon compressive molding can be accounted for by the dimensional change occurring only in one dimension. In another embodiment, substantially all of the changes in preform volume occurring upon compressive molding can be accounted for by the dimensional change occurring only in the thickness dimension. In another embodiment, all of the changes in preform volume occurring upon compressive molding can be accounted for by the dimensional change occurring only in the thickness dimension. In another embodiment, substantially all of the changes in preform volume occurring upon compressive molding can be accounted for by the dimensional change occurring only in the length or height dimension. In another embodiment, all of the changes in preform volume occurring upon compressive molding can be accounted for by the dimensional change occurring only in the length or height dimension.

The linear compression ratio, defined herein as the ratio of the original magnitude of the dimension that is reduced during compressive molding to the magnitude of the final dimension after compressive molding, is from about 1.1 to about 9.9. In another embodiment, the linear compression ratio is from about 1.5 to about 8.0. In another embodiment, the linear compression ratio is from about 2.5 to about 7.0. In another embodiment, the linear compression ratio is from about 2.0 to about 6.0.

If the reduction in the dimension that is reduced during compressive molding is expressed in terms of linear compressive strain, i.e., the change in a dimension over that original dimension, the linear compressive strain is from about 3% to about 97%. In another embodiment, the linear compressive strain is from about 15% to about 95%. In another embodiment, the linear compressive strain is from about 25% to about 90%. In another embodiment, the linear compressive strain is from about 30% to about 85%. In another embodiment, the linear compressive strain is from about 40% to about 75%.

In another embodiment, during compressive molding the radius dimension of a cylindrical preform is reduced, i.e., the circumference is reduced, such that the dimensional reduction occurs in two directions, while, in the other direction, the cylinder's height remains substantially unchanged. In another embodiment, during compressive molding the radius dimension of a cylindrical preform is reduced, while, in the other direction, the cylinder's height remains unchanged.

In another embodiment, substantially all of the changes in preform volume occurring upon compressive molding can be accounted for by the dimensional change occurring only in two dimensions. In another embodiment, all of the changes in preform volume occurring upon compressive molding can be accounted for by the dimensional change occurring only in two dimensions. In another embodiment, substantially all of the changes in preform volume occurring upon compressive molding can be accounted for by the dimensional change occurring only in the radial dimension. In another embodiment, all of the changes in preform volume occurring upon compressive molding can be accounted for by the dimensional change occurring only in the radial dimension.

The radial compression ratio, defined herein as the ratio of the original magnitude of the cylindrical preform's radius to the magnitude of the final radius after compressive molding, is from about 1.2 to about 6.7. In another embodiment, the radial compression ratio is from about 1.5 to about 6.0. In another embodiment, the radial compression ratio is from about 2.5 about 6.0. In another embodiment, the radial compression ratio is from about 2.0 to about 5.0.

In another embodiment, the cross-sectional compression ratio, defined herein as the ratio of the original magnitude of the cylindrical preform's cross-sectional area to the magnitude of the final cross-sectional area after compressive molding, is from about 1.5 to about 47. In another embodiment, the cross-sectional compression ratio is from about 1.5 to about 25. In another embodiment, the cross-sectional compression ratio is from about 2.0 to about 9.0. In another embodiment, the cross-sectional compression ratio is from about 2.0 to about 7.0.

If the reduction in the cross-sectional area during compressive molding of a cylindrical preform is expressed in terms of cross-sectional compressive strain, i.e., the change in a cross-sectional area over that original cross-sectional area, the cross-sectional compressive strain is from about 25% to about 90%. In another embodiment, the cross-sectional compressive strain is from about 33% to about 88%. In another embodiment, the cross-sectional compressive strain is from about 50% to about 88%.

Compressive molding of the biodurable reticulated elastomeric matrix materials of the present invention is conducted at temperatures above 25° C. and can be carried out from about 100° C. to about 190° C. in one embodiment, from about 110° C. to about 180° C. in another embodiment, or from about 120° C. to about 145° C. in another embodiment. In another embodiment, as the temperature at which the compressive molding process is carried out increases, the time at which the compressive molding process is carried out decreases. The time for compressive molding is usually from about 10 seconds to about 10 hours. In another embodiment, the compressive molding time is from about 30 seconds to about 5 hours. In another embodiment, the compressive molding time is from about 30 seconds to about 3 hours. As the temperature at which the compressive molding process is conducted is raised, the time for compressive molding decreases. At higher temperatures, the time for compressive molding must be short, as a long compressive molding time may cause the reticulated elastomeric matrix to thermally degrade. For example, in one embodiment, at temperatures of about 160° C. or greater, the time for compressive molding is about 30 minutes or less in one embodiment, about 10 minutes or less in another embodiment, or about 5 minutes or less in another embodiment. In another embodiment, at a temperature of about 150° C., e.g., from about 145° C. to about 155° C., the time for compressive molding is about 60 minutes or less in one embodiment, about 20 minutes or less in another embodiment, or about 10 minutes or less in another embodiment. In another embodiment, at temperatures of about 130° C., e.g., from about 125° C. to about 135° C., the time for compressive molding is about 240 minutes or less in one embodiment, about 120 minutes or less in another embodiment, or about 30 minutes or less in another embodiment.

After compressive molding, the ratio of the density of the compressed reticulated elastomeric matrix to the density of the reticulated elastomeric matrix before compressive molding can increase by a factor of from about 1.05 times to about 25 times. In another embodiment, the density of the compressed reticulated elastomeric matrix can increase by a factor of from about 1.20 times to about 7.5 times; for example, from an initial density of 3.5 lbs/ft³ (0.056 g/cc) to a density of 4.2 lbs/ft³ (0.067 g/cc) after compressive molding in one embodiment, or to a density of 26.3 lbs/ft³ (0.421 g/cc) after compressive molding in another embodiment. In another embodiment, the density of the compressed reticulated elastomeric matrix can increase, for example, from an initial density of 3.4 lbs/ft³ (0.054 g/cc) to 7.9 lbs/ft³ (0.127 g/cc) after compressive molding.

After compressive molding, the tensile strength of the compressed reticulated elastomeric matrix can increase by a factor of from about 1.05 times to about 5.0 times relative to the tensile strength of the reticulated elastomeric matrix before compressive molding. In another embodiment, the tensile strength of the compressed reticulated elastomeric matrix can increase by a factor of from about 1.20 times to about 2.5 times; for example, from an initial tensile strength of 52 psi (36,400 kg/m²) to a tensile strength of 62.4 psi (43,700 kg/m²) after compressive molding in one embodiment, or to 130 psi (91,000 kg/m²) after compressive molding in another embodiment. In another embodiment, the tensile strength of the compressed reticulated elastomeric matrix can increase, for example, from an initial tensile strength of 52 psi (36,400 kg/m²) to 120 psi (84,000 kg/m²) after compressive molding. In other embodiments, the increase in tensile strength occurs in the direction of the preferred orientation in one dimensional, two dimensional or three dimensional compressive molding.

After compressive molding, the compressive strength of the compressed reticulated elastomeric matrix can increase by a factor of from about 1.05 times to about 4.5 times relative to the compressive strength of the reticulated elastomeric matrix before compressive molding. In another embodiment, the compressive strength of the compressed reticulated elastomeric matrix can increase by a factor of from about 1.20 times to about 3.5 times; for example, from an initial compressive strength of 2.4 psi (1.700 kg/m²) at 50% compressive strain to 2.9 psi (2,000 kg/m²) at 50% compressive strain after compressive molding in one embodiment, or to 8.4 psi (5,900 kg/m²) at 50% compressive strain after compressive molding in another embodiment. In other embodiments, the increase in compressive strength occurs in the direction of the preferred orientation in one dimensional, two dimensional or three dimensional compressive molding.

After compressive molding, the permeability of the compressed reticulated elastomeric matrix usually decreases and, thereby, potentially reduces the ability of the compressed reticulated elastomeric matrix to provide for tissue ingrowth and proliferation. Therefore, it is important to maintain good permeability after compressive molding. For example, in one embodiment, the initial reticulated elastomeric matrix permeability to a fluid of at least about 450 Darcy decreases to no less than about 250 Darcy when, after compressive molding of that reticulated elastomeric matrix, the cross-sectional area is reduced by about 50%. In another embodiment, the initial reticulated elastomeric matrix permeability to a fluid of at least about 450 Darcy decreases to no less than about 100 Darcy when, after compressive molding of that reticulated elastomeric matrix, the cross-sectional area is reduced by about 60%. In another embodiment, the initial reticulated elastomeric matrix permeability to a fluid of at least about 450 Darcy decreases to no less than about 20 Darcy when, after compressive molding of that reticulated elastomeric matrix, the cross-sectional area is reduced by about 80%.

In another embodiment, the initial reticulated elastomeric matrix permeability of about 300 Darcy decreases to no less than about 100 Darcy when, after compressive molding of that reticulated elastomeric matrix, the cross-sectional area is reduced by about 50%. In another embodiment, the initial reticulated elastomeric matrix permeability to a fluid of at least about 300 Darcy decreases to no less than about 80 Darcy when, after compressive molding of that reticulated elastomeric matrix, the cross-sectional area is reduced by about 60%. In another embodiment, the initial reticulated elastomeric matrix permeability to a fluid of at least about 300 Darcy decreases to no less than about 15 Darcy when, after compressive molding of that reticulated elastomeric matrix, the cross-sectional area is reduced by about 75%.

In another embodiment, the initial reticulated elastomeric matrix permeability to a fluid of at least about 200 Darcy decreases to no less than about 40 Darcy when, after compressive molding of that reticulated elastomeric matrix, the cross-sectional area is reduced by about 50%. In another embodiment, the initial reticulated elastomeric matrix permeability to a fluid of at least about 200 Darcy decreases to no less than about 80 Darcy when, after compressive molding of that reticulated elastomeric matrix, the cross-sectional area is reduced by about 50%. In another embodiment, the initial reticulated elastomeric matrix permeability to a fluid of at least about 200 Darcy decreases to no less than about 40 Darcy when, after compressive molding of that reticulated elastomeric matrix, the cross-sectional area is reduced by about 60%. In another embodiment, the initial reticulated elastomeric matrix permeability to a fluid of at least about 200 Darcy decreases to no less than about 15 Darcy when, after compressive molding of that reticulated elastomeric matrix, the cross-sectional area is reduced by about 70%.

Reinforcement Incorporation

Elastomeric matrix 10 can undergo a further post-reticulation processing step or steps, in addition to reticulation, imparting endpore features and compressive molding already discussed above. For example, in another embodiment, the reticulated elastomeric matrix is reinforced with a reinforcement. In other embodiments, the reinforcement is in at least one dimension, e.g., a 1-dimensional reinforcement (such as a fiber), a 2-dimensional reinforcement (such as a 2-dimensional mesh made up of intersecting 1-dimensional reinforcement elements), or a 3-dimensional reinforcement (such as a 3-dimensional grid).

The reinforced elastomeric matrix and/or compressed reinforced elastomeric matrix can be made more functional for specific uses in various implantable devices by including or incorporating a reinforcement, e.g., fibers, into the reticulated cross-linked biodurable elastomeric polycarbonate urea-urethane matrix. The enhanced functionalities that can be imparted by using a reinforcement include but are not limited to enhancing the ability of the device to withstand pull out loads associated with suturing during surgical procedures, the device's ability to be positioned at the repair site by suture anchors during a surgical procedure, and holding the device at the repair site after the surgery when the tissue healing takes place. In another embodiment, the enhanced functionalities provide additional load bearing capacities to the device during surgery in order to facilitate the repair or regeneration of tissues. In another embodiment, the enhanced functionalities provide additional load bearing capacities to the device, at least through the initial days following surgery, in order to facilitate the repair or regeneration of tissues. In another embodiment, the enhanced functionalities provide additional load bearing capacities to the device following surgery in order to facilitate the repair or regeneration of tissues.

One way of obtaining enhanced functionalities is by incorporating a reinforcement, e.g., fibers, fiber meshes, wires and/or sutures, into the elastomeric matrix. Another exemplary way of obtaining enhanced functionalities is by reinforcing the matrix with at least one reinforcement. The incorporation of the reinforcement into the matrix can be achieved by various ways, including but not limited to stitching, sewing, weaving and knitting. In one embodiment, the attachment of the reinforcement to the matrix can be through a sewing stitch. In another embodiment, the attachment of the reinforcement to the matrix can be through a sewing stitch that includes an interlocking feature. In another embodiment, the incorporation of the reinforcement into the matrix can be achieved by foaming of the elastomeric matrix ingredients around a pre-fabricated or pre-formed reinforcement element made from a reinforcement and reticulating the composite structure thus-formed to create an intercommunicating and interconnected pore structure. In one embodiment, the reinforcement used does not interfere with the matrix's capacity to accommodate tissue ingrowth and proliferation.

The elastomeric matrix that incorporates the fibers into the reticulated cross-linked biodurable elastomeric polycarbonate urea-urethane matrix can vary in its density and/or in its orientation. The density of the elastomeric matrix can vary, in one embodiment from about 2 lbs/ft³ to about 25 lbs/ft (from about 0.032 g/cc to about 0.401 g/cc), from about 2.5 lbs/ft³ to about 10 lbs/ft³ (from about 0.040 g/cc to about 0.160 g/cc) in another embodiment, or from about 3 lbs/ft³ to about 8.5 lbs/ft³ (from about 0.480 g/cc to about 0.136 g/cc) in another embodiment. Orientation can occur during initial formation of foam, during reticulation, or during secondary processing that may occur after reticulation and thermal curing of the foam. The results of orientation are manifested by enhanced properties and/or enhanced performance in the direction of orientation. In one embodiment, a device made from a reinforced reticulated elastomeric matrix is positioned in the tissue being repaired in such a way that the enhanced properties and/or enhanced performance of the oriented matrix is aligned in the direction to resist the higher load bearing direction. Incorporation of the reinforcement may lead to enhanced performance of the matrix, which is superior to that which would be obtained by orienting the reinforced matrix in one or more directions.

The reinforcement can comprise mono-filament fiber, multi-filament yarn, braided multi-filament yarns, comingled mono-filament fibers, comingled multi-filament yarns, bundled mono-filament fibers, bundled multi-filament yarns, and the like. The reinforcement can comprise an amorphous polymer, semi-crystalline polymer, e.g., polyester or nylon, carbon, e.g., carbon fiber, glass, e.g., glass fiber; ceramic, cross-linked polymer fiber and the like or any mixture thereof. The fibers can be made from absorbable or non-absorbable materials. In one embodiment, the fiber reinforcement of the present invention is made from a biocompatible material(s).

In one embodiment, the reinforcement can be made from at least one non-absorbable material, such as a non-biodegradable or non-absorbable polymer. Examples of suitable non-absorbable polymers include but are not limited to polyesters (such as polyethylene terephthalate and polybutylene terephthalate); polyolefins (such as polyethylene and polypropylene including atactic, isotactic, syndiotactic, and blends thereof as well as, polyisobutylene and ethylene-alpha-olefin copolymers); acrylic polymers and copolymers; vinyl halide polymers and copolymers (such as polyvinyl chloride); polyvinyl ethers (such as polyvinyl methyl ether); polyvinylidene halides (such as polyvinylidene fluoride and polyvinylidene chloride); polyacrylonitrile; polyvinyl ketones; polyvinyl aromatics (such as polystyrene); polyvinyl esters (such as polyvinyl acetate); copolymers of vinyl monomers with each other and olefins (such as etheylene-methyl methacrylate copolymers, acrylonitrile-styrene copolymers, ABS resins and ethylene-vinyl acetate copolymers); polyamides (such as nylon 4, nylon 6, nylon 66, nylon 610, nylon 11, nylon 12 and polycaprolactam); alkyd resins; polycarbonates; polyoxymethylenes; polyimides; polyethers; epoxy resins; polyurethanes; rayon; rayon-triacetate; and any mixture thereof. Polyamides, for the purpose of this application, also include polyamides of the form —NH—(CH₂)ₙ—C(O)— and —NH—(CH₂)ₙ—NH—C(O)—(CH₂)_{y}—C(O)—, wherein n is an integer from 6 to 13 inclusive; x is an integer from 6 to 12 inclusive; and y is an integer from 4 to 16 inclusive.

In another embodiment, the reinforcement can be made from at least one biodegradable, bioabsorbable or absorbable polymer. Examples of suitable absorbable polymers include but are not limited to aliphatic polyesters, e.g., homopolymers and copolymers of lactic acid, glycolic acid, lactide, glycolide, para-dioxanone, trimethylene carbonate, ∈-caprolactone and blends thereof. Further exemplary biocompatible polymers include film-forming bioabsorbable polymers such as aliphatic polyesters, poly(amino acids), copoly(ether-esters), polyalkylenes oxalates, polyamides, poly(iminocarbonates), polyorthoesters, polyoxaesters including polyoxaesters containing amido groups, polyamidoesters, polyanhydrides, polyphosphazenes, biomolecules, and any mixture thereof. Aliphatic polyesters, for the purpose of this application, include polymers and copolymers of lactide (which includes lactic acid d-, l- and meso lactide), ∈-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, 6,6-dimethyl-1,4-dioxan-2-one, and any mixture thereof.

Such fiber(s)/yarn(s) can be made by melt extrusion, melt extrusion followed by annealing and stretching, solution spinning, electrostatic spinning, and other methods known to those in the art. Each fiber can be bi-layered, with an inner core and an outer sheath, or multi-layered, with inner core, an outer sheath and one or more intermediate layers. In bi- and multi-layered fibers, the core, the sheath or any layer(s) outside the core can comprise a degradable or dissolvable polymer. The fibers can be uncoated or coated with a coating that can comprise an amorphous polymer, semi-crystalline polymer, carbon, glass, ceramic, and the like or any mixture thereof.

The reinforcement can be made from carbon, glass, a ceramic, bioabsorbable glass, silicate-containing calcium-phosphate glass, or any mixture thereof. The calcium-phosphate glass, the degradation and/or absorption time in the human body of which can be controlled, can contain metals, such as iron, magnesium, sodium, potassium, or any mixture thereof.

In another embodiment, the 1-dimensional reinforcement comprises an amorphous polymer fiber, a semi-crystalline polymer fiber, a cross-linked polymer fiber, a biopolymer fiber, a collagen fiber, an elastin fiber, carbon fiber, glass fiber, bioabsorbable glass fiber, silicate-containing calcium-phosphate glass fiber, ceramic fiber, polyester fiber, nylon fiber, an amorphous polymer yarn, a semi-crystalline polymer yarn, a cross-linked polymer yarn, a biopolymer yarn, a collagen yarn, an elastin yarn, carbon yarn, glass yarn, bioabsorbable glass yarn, silicate-containing calcium-phosphate glass yarn, ceramic yarn, polyester yarn, nylon yarn, or any mixture thereof. In another embodiment, the 2-dimensional reinforcement comprises intersecting 1-dimensional reinforcement elements comprising an amorphous polymer fiber, a semi-crystalline polymer fiber, a cross-linked polymer fiber, a biopolymer fiber, carbon fiber, glass fiber, bioabsorbable glass fiber, silicate-containing calcium-phosphate glass fiber, ceramic fiber, polyester fiber, nylon fiber, an amorphous polymer yarn, a semi-crystalline polymer yarn, a cross-linked polymer yarn, a biopolymer yarn, carbon yarn, glass yarn, bioabsorbable glass yarn, silicate-containing calcium-phosphate glass yarn, ceramic yarn, polyester yarn, nylon yarn, or any mixture thereof.

The reinforcement can be incorporated into the reticulated elastomeric matrix in different patterns. In one embodiment, the reinforcement is placed along the border of the device, maintaining a fixed distance from the device's edges. In another embodiment, the reinforcement is placed along the border of the device, maintaining a variable distance from the device's edges. In another embodiment, the reinforcement is placed along the perimeter, e.g., circumference for a circular device, of the device, maintaining a fixed distance from the device's edges. In another embodiment, the reinforcement is placed along the perimeter of the device, maintaining a variable distance from the device's edges. In another embodiment, the reinforcement is present as a plurality of parallel and/or substantially parallel 1-dimensional reinforcement elements, e.g., as a plurality of parallel lines such as parallel fibers. In another embodiment, the reinforcement is placed as a 2- or 3-dimensional reinforcement grid in which the 1-dimensional reinforcement elements cross each other's path. The grid can have one or multiple reinforcement elements. In 2- or 3-dimensional reinforcement grid embodiments, the elements of the reinforcement can be arranged in geometrically-shaped patterns, such as square, rectangular, trapezoidal, triangular, diamond, parallelogram, circular, eliptical, pentagonal, hexagonal, and/or polygons with seven or more sides. The reinforcement elements comprising a reinforcement grid can all be of the same shape and size or can be of different shapes and sizes. The reinforcement elements comprising a reinforcement grid can additionally include border, perimeter and/or parallel line elements. The performance or properties of the reinforcement grid incorporates the reinforcement into the matrix and the thus-reinforced matrix depends on the inherent properties of the reinforcement as well as the pattern, geometry and number of elements of the grid.

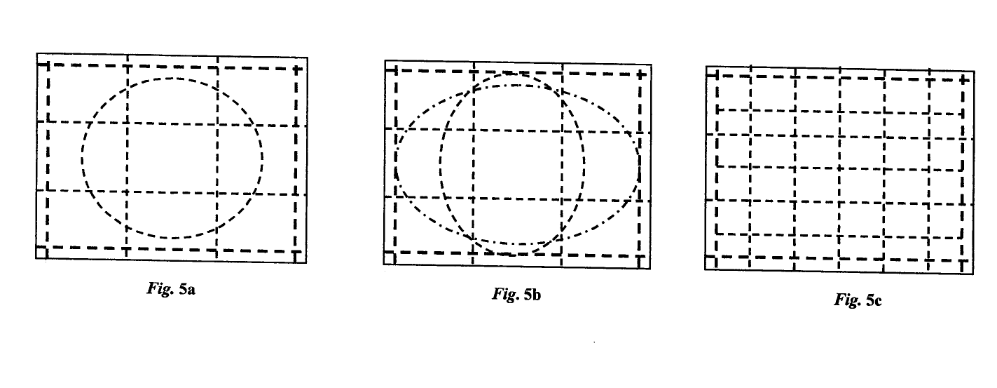

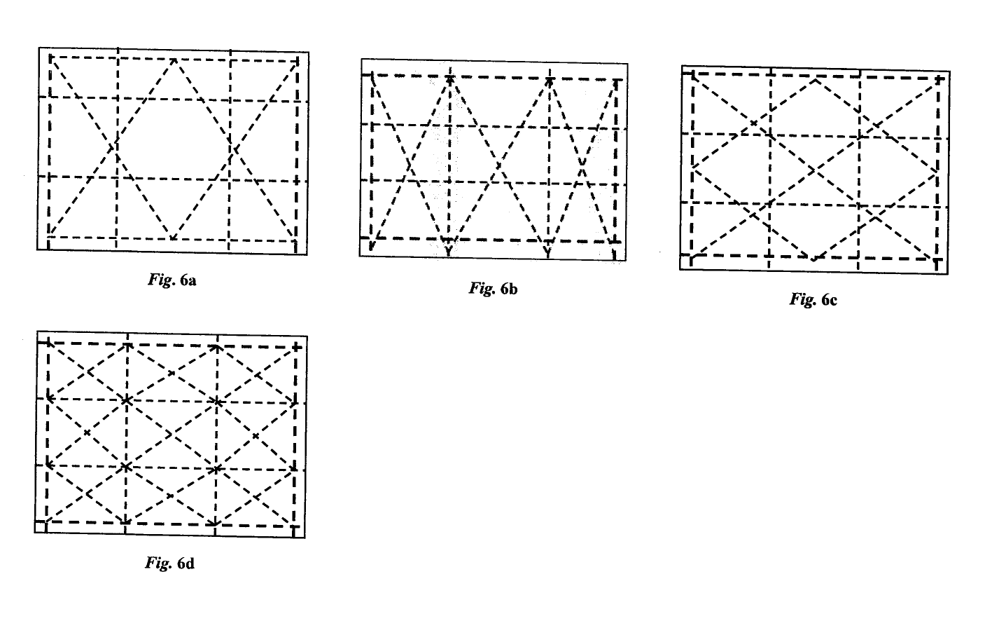

Some exemplary, but not limiting, reinforcement grids are illustrated in FIGS. 5 and 6. Each of FIGS. 5a-5c and 6a-6d include a border or perimeter reinforcing element or elements. FIG. 5a illustrates an eliptical reinforcement element superimposed on a rectangular grid reinforcement element. FIG. 5b illustrates two eliptical reinforcement elements superimposed on a rectangular grid reinforcement element. FIG. 5c illustrates a rectangular grid reinforcement element. FIG. 6a illustrates a diamond-shaped grid reinforcement element superimposed on a rectangular grid reinforcement element. FIG. 6b illustrates a 4-sided polygional-shaped grid reinforcement element superimposed on a rectangular grid reinforcement element. FIGS. 6c and 6d illustrate diamond-shaped grid reinforcement elements of different spacing and diagional reinforcement elements superimposed on a rectangular grid reinforcement element.

In one embodiment, any one of the edges of a single grid element can be from about 0.25 mm to about 20 mm long, or from about 5 mm to about 15 mm long in another embodiment.

In other embodiments, the clearance or spacing between reinforcement elements, such as the clearance between adjacent linear reinforcement elements, can be from about 0.25 mm to about 20 mm in one embodiment, or from about 0.5 mm to about 15 mm in another embodiment. In other embodiments, the clearance between reinforcement elements is substantially the same between elements. In other embodiments, the clearance between reinforcement elements differs between different elements. In other multi-dimensional reinforcement embodiments, the clearance between reinforcement elements in one dimension is independent of the clearance(s) between reinforcement elements in any other dimension.

The diameter of a reinforcement element having a substantially circular cross-section can be from about 0.03 mm to about 0.50 mm in one embodiment, or from about 0.07 mm to about 0.30 mm in another embodiment, or from about 0.05 mm to about 1.0 mm in another embodiment, or from about 0.03 mm to about 1.0 mm in another embodiment. In another embodiment, the diameter of a reinforcement element having a substantially circular cross-section can be equivalent to a USP suture diameter from about size 8-0 to about size 0 in one embodiment, from about size 8-0 to about size 2 in another embodiment, from about size 8-0 to about size 2-0 in another embodiment.

The reinforcement layout or the distribution and pattern of reinforcement elements, e.g., fibers or sutures, in the matrix will depend on design requirement and/or the application for which the device will be used. In an embodiment where sewing is used to incorporate the reinforcement into the matrix, the pitch of the stitch, i.e., the distance between successive stitches or attachment points within the same line, is from about 0.25 mm to about 4 mm in one embodiment or from about 1 mm to about 3 mm in another embodiment.

In one embodiment, in some applications, such as rotator cuff repair where the implantable device serves in an augmentary role, precise fitting may not be required to match or fit the tissue that is being repaired or regenerated. In another embodiment, an implantable device containing a reinforced reticulated elastomeric matrix is shaped prior to its use, such as in surgical repair of tendons and ligaments. One exemplary method of shaping is trimming. When shaping is desired, the reinforced reticulated elastomeric matrix can be trimmed in its length and/or width direction along the lines or reinforcing fibers. In one embodiment, this trimming is accomplished so as to leave about 2 mm outside the reinforcement border, e.g., to facilitate suture attachment during surgery.

For a device of this invention comprising a reinforced reticulated elastomeric matrix, the maximum dimension of any cross-section perpendicular to the device's thickness is from about 0.25 mm to about 100 mm in one embodiment. In another embodiment, the maximum thickness of the device is from about 0.25 mm to about 20 mm.

In one embodiment, the implantable device and/or its reinforcement can be coated with one or more bioactive molecules, such as the proteins, collagens, elastin, entactin-1, fibrillin, fibronectin, cell adhesion molecules, matricellular proteins, cadherin, integrin, selectin, H-CAM superfamilies, and the like described in detail herein.

In one embodiment, devices incorporating reinforcement into a reticulated elastomeric matrix will have at least one characteristic within the following ranges of performance. The suture pullout strength is from about 1.1 lbs/ft to about 17 lbs/ft (from about 5 Newtons to about 75 Newtons) in one embodiment or from about 2.3 lbs/ft to about 9.0 lbs/ft (from about 10 Newtons to about 40 Newtons) in another embodiment. The break strength is from about 2.0 lbs/ft to about 100 lbs/ft (from about 8.8 Newtons to about 440 Newtons) in another embodiment, or from about 3.4 lbs/ft to about 45 lbs/ft (from about 15 Newtons to about 200 Newtons) in one embodiment, or from about 6.8 lbs/ft to about 22.5 lbs/ft (from about 30 Newtons to about 100 Newtons) in another embodiment. The ball burst strength is from about 3 lbsf to about 75 lbsf (from about 1.35 Kgf to about 34 Kgf) in one embodiment or from about 8 lbsf to about 50 lbsf (from about 3.65 Kgf to about 22.5 Kgf) in another embodiment.

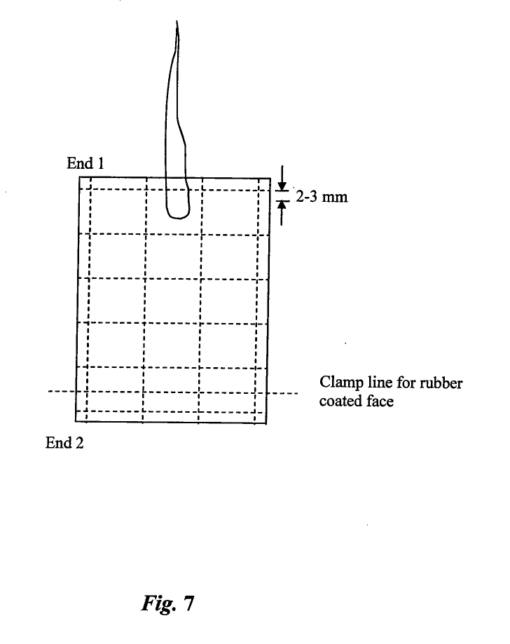

The suture pullout strength test was carried out using an INSTRON Tester (Model 3342) equipped with 1 kN pneumatic grips upper and lower gripping jaws, each having opposed 25 mm×25 mm rubber coated gripping faces. FIG. 7 illustrates the geometry of the reinforced specimen and the suture in an embodiment of the suture pullout strength test. The test suture wais a length of 2-0 ETHIBOND braided polyester suture. After the instrument's gauge length was set to 60 mm (2.36 inches), one end (End 2) of the reinforced reticulated elastomeric matrix device to be tested was clamped into the instrument's lower fixed jaw. The ETHIBOND test suture was inserted into the other end (End 1) of the reinforced reticulated elastomeric matrix device by using a needle. A loop was formed by the two ends of the test suture strands. The test suture was attached to the reinforced device 2 to 3 mm below the horizontal reinforcement line closest to the device's edge and, preferably, towards the center of the device's width, as illustrated in FIG. 7 for a device reinforced with a rectangular grid of fibers.

The free ends of the test suture were about 50 to 60 mm in length from the point where the test suture was attached to the reinforced reticulated elastomeric matrix device. The free ends of the suture were clamped into the instrument's upper movable jaw. Thereafter, the suture retention strength test was run at a rate of 100 mm/min (3.94 in/min) with the movable jaw moving upwards and away from the fixed jaw. The maximum force reached in the force-extension curve was noted as the suture retention strength, provided that the tear in the reinforced reticulated elastomeric matrix device was limited to the area near the End 1 horizontal grid line that was adjacent to the suture attachment position. The mean and standard deviation were determined from testing of a plurality of samples.

The break strength test was carried out in the same way as the suture pullout strength test described above except that the braided polyester suture is not used and the reinforced reticulated elastomeric matrix device to be tested was clamped between the instrument's lower fixed jaw and the upper movable jaw. Thereafter, the break strength test was run at a rate of 100 mm/min (3.94 in/min) with the movable jaw moving upwards and away from the fixed jaw. The maximum force reached in the force-extension curve was noted as the break strength.

The ball burst strength was measured pursuant to the test method described in ASTM Standard 3787 except that a smaller ball with a diameter of 10 mm, an 18 mm diameter retaining hole, and a crosshead speed of 102 mm/min (4 inch/min) were used.

Other Post-Processing of the Reticulated Elastomeric Matrix

Elastomeric matrix 10 can undergo a further processing step or steps, in addition to those already discussed above. For example, elastomeric matrix 10 or the products made from elastomeric matrix 10 can be annealed to stabilize the structure.

In one embodiment, annealing at elevated temperatures can promote increased crystallinity in polyurethanes. In another embodiment, annealing at elevated temperatures can also promote structural stabilization in cross-linked polyurethanes and long-term shelf-life stability. The structural stabilization and/or additional crystallinity can provide enhanced shelf-life stability to implantable-devices made from elastomeric matrix 10. In one embodiment, without being bound by any particular theory, annealing leads to relaxation of the stresses formed in the reticulated elastomeric matrix structure during foam formation and/or reticulation.

In one embodiment, annealing is carried out at temperatures in excess of about 50° C. In another embodiment, annealing is carried out at temperatures in excess of about 100° C. In another embodiment, annealing is carried out at temperatures in excess of about 125° C. In another embodiment, annealing is carried out at temperatures of from about 100° C. to about 135° C. In another embodiment, annealing is carried out at temperatures of from about 100° C. to about 130° C. In another embodiment, annealing is carried out at temperatures of from about 100° C. to about 120° C. In another embodiment, annealing is carried out at temperatures of from about 105° C. to about 115° C.

In another embodiment, annealing is carried out for at least about 2 hours. In another embodiment, annealing is carried out for from about 2 to about 15 hours. In another embodiment, annealing is carried out for from about 3 to about 10 hours. In another embodiment, annealing is carried out for from about 4 to about 8 hours.

Annealing can be carried out with or without constraining the device. In another embodiment, the elastomeric matrix 10 is geometrically unconstrained while it is annealed, e.g., the elastomeric matrix is not surrounded by a mold. In another embodiment, the elastomeric matrix 10 is geometrically constrained while it is annealed, e.g., the elastomeric matrix is constriained by a surface, such as a mold surface, on one or more sides so that its dimension(s), such as its thickness, does not change substantially during annealing. In this embodiment, the elastomeric matrix 10 is not compressed to any significant extent by its constraint, thus, such annealing differs from compressive molding in this respect.

In one embodiment, compressive molding can be optionally followed by further annealing of the (already) compressed reticulated elastomeric matrix at a temperature of from about 110° C. to about 140° C. and for a time period of from about 15 minutes to about 4 hours. As with compressive molding, annealing can be carried while restraining the compressed matrix in a mold or without a mold. In another embodiment, annealing can be carried while restraining the compressed matrix in a mold. If the initial compressive molding occurred at a temperature or about 150° C. or greater, the time for annealing should be short so as to avoid potential for thermal degradation of the compressed reticulated elastomeric matrix at long annealing times. For example, compressive molding at a temperature of about 150° C. or greater can be followed by annealing of the compressed reticulated elastomeric matrix at a temperature of from about 125° C. to about 135° C. for a time period of from about 30 minutes to about 3 hours.

Elastomeric matrix 10 may be molded into any of a wide variety of shapes and sizes during its formation or production. The shape may be a working configuration, such as any of the shapes and configurations described in the applications to which priority is claimed, or the shape may be for bulk stock. Stock items may subsequently be cut, trimmed, punched or otherwise shaped for end use. The sizing and shaping can be carried out by using a blade, punch, drill or laser, for example. In each of these embodiments, the processing temperature or temperatures of the cutting tools for shaping and sizing can be greater than about 100° C. In another embodiment, the processing temperature(s) of the cutting tools for shaping and sizing can be greater than about 130° C. Finishing steps can include, in one embodiment, trimming of macrostructural surface protrusions, such as struts or the like, which can irritate biological tissues. In another embodiment, finishing steps can include heat annealing. Annealing can be carried out before or after final cutting and shaping.

Shaping and sizing can include custom shaping and sizing to match an implantable device to a specific treatment site in a specific patient, as determined by imaging or other techniques known to those in the art. In particular, one or a small number, e.g. less than about 6 in one embodiment and less than about 2 in another embodiment, of elastomeric matrices 10 can comprise an implantable device system for treating damaged tissue requiring repair and/or regeneration.

The dimensions of the shaped and sized devices made from elastomeric matrix 10 can vary depending on the particular tissue repair and regeneration site treated. In one embodiment, the major dimension of a device prior to being compressed and delivered is from about 0.5 mm to about 500 mm. In another embodiment, the major dimension of a device prior to being compressed and delivered is from about 10 mm to about 500 mm. In another embodiment, the major dimension of a device prior to being compressed and delivered is from about 50 mm to about 200 mm. In another embodiment, the major dimension of a device prior to being compressed and delivered is from about 30 mm to about 100 mm. Elastomeric matrix 10 can exhibit compression set upon being compressed and transported through a delivery-device, e.g., a catheter, syringe or endoscope. In another embodiment, compression set and its standard deviation are taken into consideration when designing the pre-compression dimensions of the device.

In one embodiment, a patient is treated using an implantable device or a device system that does not, in and of itself, entirely fill the target cavity or other site in which the device system resides, in reference to the volume defined within the entrance to the site. In one embodiment, the implantable device or device system does not entirely fill the target cavity or other site in which the implant system resides even after the elastomeric matrix pores are occupied by biological fluids or tissue. In another embodiment, the fully expanded in situ volume of the implantable device or device system is at least 1% less than the volume of the site. In another embodiment, the fully expanded in situ volume of the implantable device or device system is at least 15% less than the volume of the site. In another embodiment, the fully expanded in situ volume of the implantable device or device system is at least 30% less than the volume of the site.

In another embodiment, the fully-expanded in situ volume of the implantable device or device system is from about 1% to about 40% larger than the volume of the cavity. In another embodiment, the fully-expanded in situ volume of the implantable device or device system is from about 5% to about 25% larger than the volume of the cavity. In another embodiment, the ratio of implantable device volume to the volume occupied by the orthopedic application site is from about 70% to about 90%. In another embodiment, the ratio of implantable device volume to the volume occupied by the orthopedic application site is from about 90% to about 100%. In another embodiment, the ratio of implantable device volume to the volume occupied by the orthopedic application site is from about 90% to less than about 100%. In another embodiment, the ratio of implantable device volume to the volume occupied by the orthopedic application site is from about 100% to about 140%. In another embodiment, the ratio of implantable device volume to the volume occupied by the orthopedic application site is from about 100% to about 200%. In another embodiment, the ratio of implantable device volume to the volume occupied by the orthopedic application site is from about 100% to about 300%.

Biodurable reticulated elastomeric matrices 10, or an implantable device system comprising such matrices, can be sterilized by any method known to the art including gamma irradiation, autoclaving, ethylene oxide sterilization, infrared irradiation and electron beam irradiation. In one embodiment, biodurable elastomers used to fabricate elastomeric matrix 10 tolerate such sterilization without loss of useful physical and mechanical properties. The use of gamma irradiation can potentially provide additional cross-linking to enhance the performance of the device.

In one embodiment, the sterilized products may be packaged in sterile packages of paper, polymer or other suitable material. In another embodiment, within such packages, elastomeric matrix 10 is compressed within a retaining member to facilitate its loading into a delivery-device, such as a catheter or endoscope, in a compressed configuration. In another embodiment, elastomeric matrix 10 comprises an elastomer with a compression set enabling it to expand to a substantial proportion of its pre-compressed volume, e.g., at 25° C., to at least 50% of its pre-compressed volume. In another embodiment, expansion occurs after elastomeric matrix 10 remains compressed in such a package for typical commercial storage and distribution times, which will commonly exceed 3 months and may be up to 1 or 5 years from manufacture to use.

Radio-Opacity

In one embodiment, implantable device can be rendered radio-opaque to facilitate in vivo imaging, for example, by adhering to, covalently bonding to and/or incorporating into the elastomeric matrix itself particles of a radio-opaque material. Radio-opaque materials include titanium, tantalum, tungsten, barium sulfate or other suitable material known to those skilled in the art.

Implantable Device Uses

Implantable device systems incorporating reticulated elastomeric matrix can be used as described in the applications to which priority is claimed. In one embodiment, implantable devices comprising reticulated elastomeric matrix can be used to treat a tissue defect, e.g., for the repair, reconstruction, regeneration, augmentation, gap interposition or any mixture thereof in an orthopedic application, general surgical application, cosmetic surgical application, tissue engineering application, or any mixture thereof.

In another embodiment, implantable devices comprising reticulated elastomeric matrix can be used in an orthopedic application for the repair, reconstruction, regeneration, augmentation, gap interposition or any mixture thereof of tendons, ligaments, cartilige, meniscus, spinal discs or any mixture thereof. For example, implantable devices comprising reticulated elastomeric matrix can be used in a wide range of orthopedic applications, including but not limited to repair and regeneration encompassing the spine, shoulder, elbow, wrist, hand, knee, ankle, or other joints, as discussed in detail in priority applications. The implantable device made from biodurable reticulated elastomeric matrix provides a scaffold for tissue ingrowth which is particularly effective in treating so-called soft-tissue orthopedic disorders, e.g., attachment, regeneration, augmentation or support of soft tissues including tendon augmentation, repair of articular cartilage, meniscal repair and reconstruction, ligament reconstruction, stabilization of a herniated disc, and as a substrate for both nucleus replacement and annulus repair.

Examples of ligaments in the shoulder area that can be repaired or regenerated by the use of an implantable device comprising reticulated elastomeric matrix include the acromioclavicular ligament, glenohumeral ligament, coracohumeral ligament, tranverse humeral ligament, coracoacromial ligament, and the like. Examples of tendons in the shoulder area that can be repaired or regenerated by the use of an implantable device comprising reticulated elastomeric matrix include the supraspinatus, infraspinatus, tendon of long head of biceps brachil, and the like. Cartilage in the shoulder area can also be repaired or regenerated by the use of an implantable device comprising reticulated elastomeric matrix.

Examples of ligaments in the elbow area that can be repaired or regenerated by the use of an implantable device comprising reticulated elastomeric matrix include the medial collateral ligament (“MCL”), lateral collateral ligament, and annular ligament. Examples of tendons in the elbow area that can be repaired or regenerated by the use of an implantable device comprising reticulated elastomeric matrix include the biceps and triceps tendons. Cartilage in the elbow area that can also be repaired or regenerated by the use of an implantable device comprising reticulated elastomeric matrix.

Examples of ligaments in the knee area that can be repaired or regenerated by the use of an implantable device comprising reticulated elastomeric matrix include the posterior cruciate ligament, anterior cruciate ligament (“ACL”), patellar ligament, fibular collateral ligament, tibial collateral ligament, posterior meniscofemural ligament, posterior superior tibiofibular ligament, and the like. Examples of tendons in the knee area that can be repaired or regenerated by the use of an implantable device comprising reticulated elastomeric matrix include the quadriceps tendons. Articular cartilage in the knee area can also be repaired or regenerated by the use of an implantable device comprising reticulated elastomeric matrix.

Examples of ligaments in the ankle area that can be repaired or regenerated by the use of an implantable device comprising reticulated elastomeric matrix include the transverse crural, cruciate crural, laciniate, and the like. Examples of tendons in the ankle area that can be repaired or regenerated by the use of an implantable device comprising reticulated elastomeric matrix include the peronaei longus, peronaei brevis, Achilles tendon, and the like. Cartilage in the ankle area can also be repaired or regenerated by the use of an implantable device comprising reticulated elastomeric matrix.

In general, any ligaments, tendons and/or cartilage of the spine, shoulder, elbow, wrist, hand, knee, ankle, or other bodily joints may be repaired or regenerated by use of an implantable device comprising reticulated elastomeric matrix.

In one embodiment, an implantable device comprising reticulated elastomeric matrix is appropriately shaped to form a closure device to seal the access opening in the annulus resulting from a discotomy in order to reinforce and stabilize the disc annulus in case of herniated disc, also known as disc prolapse or a slipped or bulging disc. The closure device can be compressed and delivered into the annulus opening by a cannula used during the discectomy procedure. The device can be secured into the opening by at least the following two mechanisms. First, the outwardly resilient nature of the reticulated solid phase 12 can provide a mechanical means for preventing migration. Second, the reticulated solid phase 12 can serve as a substrate to support fibrocartilage growth into the interconnected void phase 14 of the elastomeric matrix. Additional securing may be obtained by the use of anchors, sutures or biological glues and adhesives, as known to those in the art. The closure device can support fibrocartilage ingrowth into the elastomeric matrix of the implantable device.

In another embodiment, an implantable device comprising reticulated elastomeric matrix is fabricated into a patch which can be anchored, e.g., by suturing, anchors, staples and the like, into place to provide support to tendons while they heal, allowing for in-situ tendon augmentation and reinforcement. This is particularly useful for rotator cuff or bankart repair where the tendon tissue has deteriorated or developed a chronic defect and the remaining tendon is not strong enough to hold the necessary sutures for successful anchoring of tendons, where the tendons and muscles have contracted and cannot be stretched enough for reattachment (retracted tendons), or for tendons, muscles or tissues that have ruptured from an injury. The implantable device comprising reticulated elastomeric matrix can serve as a substrate for tissue ingrowth to augment the tendon and provide support during the healing process. In one embodiment, the implantable device comprising reticulated elastomeric matrix can serve as a gap interposition or a bridge to repair fully or partially torn ligaments or tendons by providing a site for repair and also a substrate for tissue ingrowth. Such an implantable device can also allow for repair of inoperable tendons that could not otherwise be reconnected. The implantable device comprising reticulated elastomeric matrix can be used for MCL repair. The implantable device can be affixed atop the repair site (underneath the ligament) using conventional suturing or fixed onto bones (medial femoral condyle or medial tibial plature) using permanent, e.g., metallic, or so-called bio-resorbable staples or anchors/sutures. The patch can also be attached with a bio-glue to the intended repair site (such as tendon, ligament or dura) as an augmentation device.

In another embodiment, reticulated elastomeric matrix or the implantable device comprising reticulated elastomeric matrix is fabricated into a biodurable substrate that, when implanted in an acellular mode, supports tissue repair and regeneration of articular cartilage, thereby having utility in knee injury treatment, e.g., for meniscal repair and ACL reconstruction. The implantable device comprising reticulated elastomeric matrix can be shaped like the medial or lateral meniscus. The implantable device comprising reticulated elastomeric matrix can be used for a total meniscus or partial meniscus replacement. The total meniscus or a segment of the meniscus can be sutured or stapled to the bone or adjacent meniscus tissue.

Another use of the implantable device comprising reticulated elastomeric matrix is for repair of weakness in biologic connective tissue that allows the bulging or herniation of another organ or organ system(s) with the resultant physiologic impairment. In one embodiment, the features of the implantable device and its functionality make it suitable for general surgical applications, such as in the repair of a hernia.

Hernias can be generally described as inguinal location or ventral abdominal with other less common but well-know variant locations, i.e., femoral or umbilical. In one embodiment, the hernea to be repaired is an inguinal hernea, a ventral abdominal hernea, a femoral hernea, an umbilical hernea, or any mixture thereof. Hernias located in the anterior or lateral abdominal wall at sites of prior surgery or trauma can be approached directly or via laproscopic approach. The repair essentially places the implantable device comprising reticulated elastomeric matrix within the abdominal wall, thereby augmenting or reinforcing defects in the muscle/facia of the rectus sheath-transversalis, external oblique and/or internal oblique. In one embodiment, the implantable device comprising the reticulated elastomeric matrix can have one side treated to be microporous or smooth on the abdominal cavity-facing side and another porous side for tissue ingrowth into the externally-facing implant.

Inguinal hernia can be approached via a pre-peritoneal approach, i.e., using the internal ring as direct access to the preperitoneal space through an open anterior approach with “tension-free” Lichenstein or plugging or, alternatively, a laproscopic approach.

In Lichtenstein tension-free repair, the inguinal canal is approached from an open anterior approach after dividing the skin, scarpa fascia, and external oblique aponeurosis. The cord is examined for an indirect sac, any direct hernia is reduced, and the floor is reinforced by an implantable device comprising reticulated elastomeric matrix being sewn to the conjoint tendon and the shelving edge of the inguinal ligament. The implantable device comprising reticulated elastomeric matrix can be slit or designed to accommodate the cord structures. In the Kugel technique, a single or bilayer of an implantable device comprising reticulated elastomeric matrix (with or without a self-retaining outer memory recoil ring) is placed anteriorly through a 4 cm muscle-splitting incision in the preperitoneal space.

The two common laparoscopic techniques include the transabdominal preperitoneal repair (“TAPP”) and the total extraperitoneal repair (“TEP”). Both the TAPP and TEP can place an implantable device comprising reticulated elastomeric matrix in the preperitoneal space. The TAPP repair is performed from within the abdomen with an incision that is made in the peritoneum to access the preperitoneal space. In the TEP repair, dissection is initiated totally in the extraperitoneal space. Goals of appropriate repair in both approaches include: (1) dissection of the myo-pectineal-orifice (MPO) and surrounding structures completely, with full exposure of the pubic bone medially and the space of Retzius; (2) removal of preperitoneal fat and cord lipomas; (3) assessment of all potential hernia sites; (4) full reduction of direct hernia sac; and (5) skeletonization of the cord to ensure proximal reduction of the indirect sac from the vas deferens and gonadal vessels.

In another embodiment, the implantable device comprising reticulated elastomeric matrix is used for cosmetic surgical applications including maxillofacial, cranial, breast, urologic, gastroesophageal or other reconstructive purposes. In such applications, the reticulated elastomeric matrix can act as a space-occupying filler and provides a scaffold for tissue ingrowth which is particularly effective in treating such plastic reconstructive disorders.

In one embodiment, an implantable device comprising reticulated elastomeric matrix is specifically designed for plastic and reconstructive surgeries such as breast soft tissue augmentation and prevention of capsule formation. Given the unique biodurable/biocompatibile nature of the present reticulated elastomeric matrix, it is particularly useful in plastic surgery of the breast. Its use can decrease the formation of implant encapsulation. Breast implants are commonly placed in surgically created pockets either beneath the breast itself or beneath the muscle underlying the breast. Breast implants (even those with textured surfaces) will form a thick solid fibrous capsule or tissue deformation (folds/creases) in up to 25% of all cases. These capsules (usually classified as 3 or 4 on a scale of 1-4 with 4 being “worst”) present a serious clinical challenge for the patient and the plastic surgeon. It is well-accepted from animal models and clinical experience that previous polyurethane foam coverings were successful in obviating and or significantly attenuating capsule formation; however, those polyurethane foam coverings were otherwise disadvantageous. In contrast, implantable devices comprising reticulated elastomeric matrix are used to obviate and or significantly attenuate capsule formation.

The implantable device can be used in several different configurations. For example, an embodiment square or rectangular in nature can be used with standard surgical fixation with care to include the fiber reinforcement in the tissue coaptation. An example of the this would be for lateral infra-mammary fold in breast reconstruction with a standard breast implant underneath the chest wall musculature. Another exemplary configuration is the implantable device as an overlay to a sub-glandular or sub-muscular breast implant. An implantable device with reinforcement mesh can be custom tailored or have existing lips on its periphery to overlap seamlessly with the standard breast implant. Implantation can be on the externally-facing side, or both sides, to increase tissue ingrowth, stabilize the implant and, moreover, attenuate or even prevent the formation of an organized thickened implant fibrous capsule.

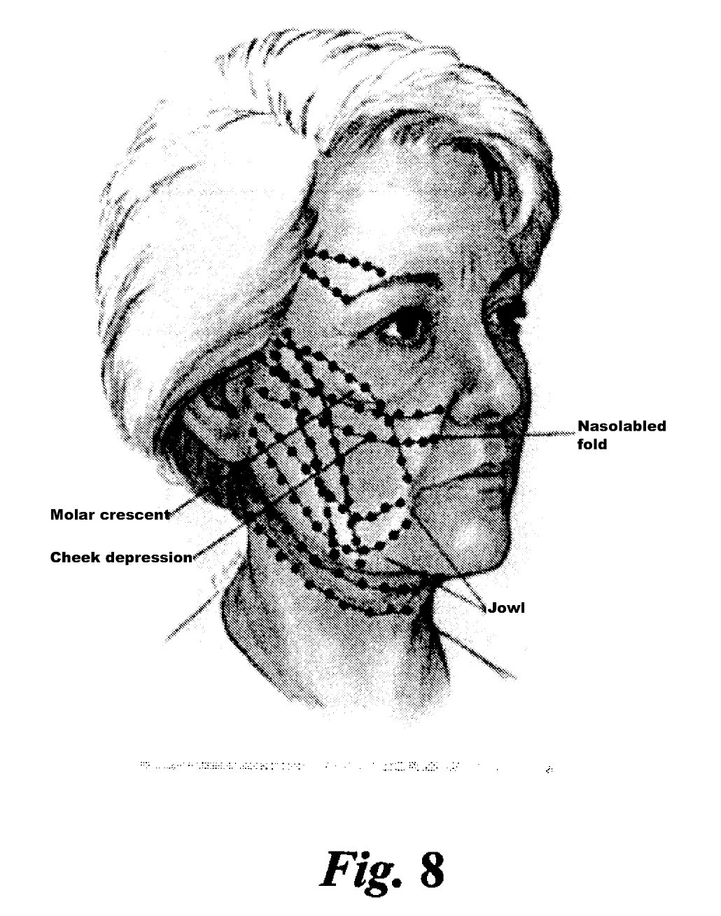

In another embodiment, the implantable device is used in cosmetic facial surgery for minimally invasive and other reconstructive applications. In facial cosmetic use, the implantable device can be passed into the supporting fascial soft tissue with a troacar or other introducer. The implantable device comprising reticulated elastomeric matrix engages the tissue throughout its course and over time the attachment, e.g., resorbable sutures, anchors, barbs, pins, screws, staples, plates, tacks, glue and the like, dissipates and the implantable device supports tissue ingrowth, thereby accomplishing secure biologic fixation. Specific regions of the forehead, midface and neck, such as the nasolabial fold, malar crescent, cheek depression and jowl illustrated in FIG. 8, can most commonly be addressed and approached via an open or minimally invasive/percutaneous technique.

An implantable device of the present invention has general use in all surgical fields where permanent biologic fixation and/or suspension, accomplished by the tissue ingrowth to the reticulated elastomeric matrix, is desirable as well.

Implantable devices comprising reticulated elastomeric matrix are also useful as a support in vitro cell propagation applications in, for example, orthopedic applications such as tissue attachment, regeneration, augmentation or support of tendons, ligaments, meniscus and annulus, and in the growth of prosthetic organ tissue.

In one embodiment, the implantable device can coantain cells, growth factors and nutrients. In another embodiment, the biodurable implantable device can serve as a template for non-autologous cells or autologous cells harvested from a patient, either of which can be cultured in an ex-vivo laboratory setting and then implanted into the patient's defect. In another embodiment, the ability of the implantable device to incorporate osteoinductive agents, such as growth factors, e.g., autologous growth factors derived from platelets and white blood cells, enables it to be functionalized in order to modulate cellular function and proactively induce tissue ingrowth. The implantable device thus provides a basis for cell therapy applications to support tissue repair and regeneration of a wide range of soft tissues including, but not limited to, articular cartilage, meniscal repair, and ACL reconstruction. The resulting implantable device fills cartilage defects, supports autologous tissue repair and regeneration, and enables subsequent integration into the repair or regeneration site, e.g., a damaged knee.

In another embodiment, the implantable device is useful in tissue engineering applications including the creation of prosthetic organ tissues, e.g., for the regeneration of liver, kidney or breast tissues.

In one non-limiting example, one or more implantable devices comprising reticulated elastomeric matrix is selected for a given site such as a target tissue healing site. The implantable device (or devices) is loaded into a delivery-device, such as a catheter, endoscope, canula, trocar or the like. In one embodiment, the delivery-device is used to deliver the implantable device comprising reticulated elastomeric matrix using minimally invasive means. After the implantable device is released from the delivery-device, it can be anchored in place so as to resist migration from the target repair or regeneration site. Methods for securing the implantable device in place include using sutures, anchors, barbs, pins, screws, staples, plates, tacks, glue, or any mixture thereof to afix the implantable device to the target repair site. The implantable device comprising reticulated elastomeric matrix can be rolled over and inserted through arthroscopic cannula into joints. In one embodiment, the implantable device is oversized compared to the target tissue healing site and resides or is held in position at the site through a compression fit, e.g., by the resilience of the reticulated elastomeric matrix. In one embodiment, an oversized implantable device conformally fits the tissue defect. Without being bound by any particular theory, the resilience and recoverable behavior that leads to such a conformal fit results in the formation of a tight boundary between the walls of the implantable device and the defect with substantially no clearance, thereby providing an interface conducive to the promotion of cellular ingrowth and tissue proliferation. Once released at the site, the implantable device comprising reticulated elastomeric matrix expands resiliently to about its original size and shape subject, of course, to any compression set limitation and any desired flexing, draping or other conformation to the site anatomy and/or geometry that the elasticity of the implantable device allows it to adopt. In another embodiment, the implantable device is inserted by an open surgical procedure.

In another embodiment; reticulated elastomeric matrix 10 is mechanically fixed to a lesion. The lesion may have resulted due to an injury or disease or may have been surgically created. The reticulated elastomeric matrix can be located within, adjacent to and/or covering the target lesion. The reticulated elastomeric matrix can serve as a defect filler, replacement tissue, tissue reinforcement and/or augmentation patch. In another embodiment, the reticulated elastomeric matrix can span defects and serve as to bridge a gap in the native tissue.

Although the implantable device comprising reticulated elastomeric matrix can be attached to the tissue repair or regeneration site by a number of different standard or acceptable surgical methods, two exemplary methods are described below. The procedures can be applied to other repair, regeneration and reconstructive procedures.

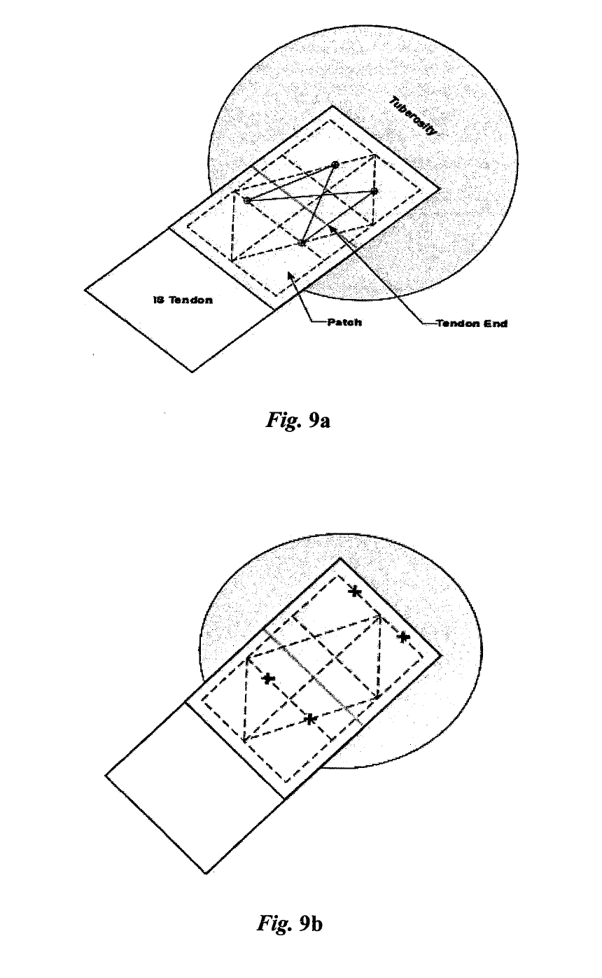

The soft tissue repair site, such as a damaged infraspinatus tendon, is decorticated with a Hall orthopedic burr. A standard area of bone is decorticated. Four Biosuture tack anchors are placed in a square configuration in the tuberosity. The infraspinatus tendon is grasped and reattached to the proximal humerus using two suture anchors and a Mason-Allen pattern stitch. The implantable device is placed on the top of the repaired site so that there is about a 0.5 cm to 2 cm overhang on the tuberosity side. The remainder of the device extends onto the tendon. The anchor sutures used for the tendon attachment will also go through the device with vertical mattress stitches and fix the device atop the repaired tendon, creating a layered construct consisting of implantable device and tendon. Laterally, the other two anchor sutures go through the device and tie it down to the tuberosity. In one embodiment, the device fixation stitches are made inside the reinforcement, e.g., inside of a reinforcement element(s) placed along the device's perimeter and/or inside the outermost element of a reinforcement grid. Four anchor suture ends will cross-over as shown in FIG. 9a.

In another embodiment, the repair proceeds as described above except that the implantable device is placed on the top of the repair site so that there is about 1 cm overhang on the tuberosity side. The remainder of the implantable device extends onto the tendon. The anchor sutures used for the tendon attachment go through the device as described above. Laterally, the other two anchor sutures go through the device as described above and tie it down to the tuberosity. The device fixation stitches are made inside the device reinforcement as shown in FIG. 9b.

In one embodiment, implantable devices made from biodurable reticulated elastomeric matrix provide an excellent scaffold for tissue ingrowth. In another embodiment, cellular entities such as fibroblasts and tissues can invade and grow into the implantable device comprising reticulated elastomeric matrix. In due course, such ingrowth can extend into the interior pores 20 and interstices of the inserted reticulated elastomeric matrix 10. Eventually, the implantable device comprising reticulated elastomeric matrix can become substantially filled with regenerating cellular ingrowth that provides a mass that can occupy the site or the void spaces in it. The types of tissue ingrowth possible include, but are not limited to, fibrous tissues, endothelial tissues, and orthopedic soft tissues.

In another embodiment, the implantable device promotes cellular ingrowth and tissue regeneration throughout the site, throughout the site boundary, or through some of the exposed surfaces, thereby sealing the site. Over time, this induced fibrovascular entity resulting from tissue ingrowth can promote the incorporation of the implantable device into the target tissue healing site. In one embodiment, this induced fibrovascular entity resulting from tissue ingrowth can cause the implantable device to be at least partially, if not substantially fully, biointegrated into the target tissue healing site. In another embodiment, tissue ingrowth can lead to repair of damaged tissues or regenerate and/or reconstruct damaged tissues. In yet another embodiment, tissue ingrowth can lead to effective resistance to migration of the implantable device over time. It may also fill the void space or defect. In another embodiment, the tissue ingrowth is scar tissue which can be long-lasting, innocuous and/or mechanically stable. In another embodiment, over the course of time, for example for 2 weeks to 3 months to 1 year, implanted reticulated elastomeric matrix 10 becomes completely filled and/or encapsulated by tissue, fibrous tissue, scar tissue or the like.

In another embodiment, an implantable device is also biocompatible, a useful characteristic for permanent biological implantation. Biocompatibility includes, but is not limited to, a demonstrated lack of carcinogenicity, mutagenicity, teratogenicity, cytotoxicity or other adverse biological effects.

In another embodiment, the properties of the implantable device comprising reticulated elastomeric matrix are engineered to be compatible with, e.g., to mimic, the tissue that is being targeted or to meet the particular requirements of a specific application. The properties of the reticulated elastomeric matrices can be engineered by controlling, e.g., the amount of cross-linking, amount of crystallinity, chemical composition, curing conditions, degree of reticulation and/or post-reticulation processing, such as annealing, compressive molding and/or incorporating reinforcement. Unlike biodegradable polymers, a reticulated elastomeric matrix maintains its physical characteristics and performance in vivo over long periods of time. Thus, it does not initiate undesirable tissue response as is observed for biodegradable implants when they break down and degrade. The high void content and degree of reticulation of a reticulated elastomeric matrix allows for tissue ingrowth and proliferation of cells within the matrix. In one embodiment, the ingrown tissue and/or regenerated cells occupy from about 25% to about 99% of the volume of interconnected void phase 14 of the original implantable device, from about 51% to about 99% in another embodiment, thereby providing the functionality, such as load bearing capability, of the original tissue that is being repaired or replaced.

In one non-limiting example, the compression set, resilience and/or recovery of the implantable device is engineered to provide high recovery force of the reticulated elastomeric matrix after repetitive cyclic loading. Such a feature is particularly advantageous in orthopedic uses in which cylic loading of the implantable device might otherwise permanently compress the reticulated elastomeric matrix, thereby preventing it from achieving the substantially continuous contact with the surrounding soft tissues necessary to permit optimal cellular infiltration and tissue ingrowth. In another non-limiting example, the density and pore size of an implantable device is engineered to provide acceptable permeability of the reticulated elastomeric matrix under compression. Such features are advantageous in spine and knee orthopedic applications, in which high loads are placed on the implantable device. In yet another non-limited example, the properties of the reticulated elastomeric matrix are engineered to maximize its “soft, conformal fit,” particularly advantageous in cosmetic surgical applications. In a further, non-limiting example, the tensile properties of the implantable device are maximized to complement the fixation technique used, e.g., to provide maximum resistance to suture pullout.

In a further embodiment, the implantable devices disclosed herein can be used as a drug delivery vehicle. For example, a therapeutic agent can be mixed with, covalently bonded to, adsorbed onto and/or absorbed into the biodurable solid phase 12. Any of a variety of therapeutic agents can be delivered by the implantable device, for example, those therapeutic agents previously disclosed herein.

### EXAMPLES

The following examples are set forth to assist in understanding the invention and should not be construed as specifically limiting the invention described herein. Such variations of the invention, including the substitution of all equivalents now known or later developed, which would be within the purview of those skilled in the art, and changes in formulation or changes in experimental design, are to be considered to fall within the scope of the invention incorporated herein.

### Example 1

### Fabrication of Cross-Linked Polyurethane Matrix 1

The aromatic isocyanate RUBINATE 9258 (from Huntsman) was used as the isocyanate component. RUBINATE 9258 is a liquid at 25° C. RUBINATE 9258 contains 4,4′-MDI and 2,4′-MDI and has an isocyanate functionality of about 2.33. A diol, poly(1,6-hexanecarbonate) diol (POLY-CD CD220 from Arch Chemicals) with a molecular weight of about 2,000 Daltons was used as the polyol component and was a solid at 25° C. Distilled water was used as the blowing agent. The blowing catalyst used was the tertiary amine triethylenediamine (33% in dipropylene glycol; DABCO 33LV from Air Products). A silicone-based surfactant was used (TEGOSTAB BF 2370 from Goldschmidt). A cell-opener was used (ORTEGOL 501 from Goldschmidt). The viscosity modifier propylene carbonate (from Sigma-Aldrich) was present to reduce the viscosity. The proportions of the components that were used is given in Table 2.

TABLE 2

| Ingredient | Parts by Weight |
|---|---|
| Polyol Component | 100 |
| Viscosity Modifier | 5.80 |
| Surfactant | 1.10 |
| Cell Opener | 1.00 |
| Isocyanate Component | 62.42 |
| Isocyanate Index | 1.00 |
| Distilled Water | 3.39 |
| Blowing Catalyst | 0.53 |

The polyol component was liquefied at 70° C. in a circulating-air oven, and 100 g thereof was weighed out into a polyethylene cup. 5.8 g of viscosity modifier was added to the polyol component to reduce the viscosity and the ingredients were mixed at 3100 rpm for 15 seconds with the mixing shaft of a drill mixer to form “Mix-1”. 1.10 g of surfactant was added to Mix-1 and the ingredients were mixed as described above for 15 seconds to form “Mix-2”. Thereafter, 1.00 g of cell opener was added to Mix-2 and the ingredients were mixed as described above for 15 seconds to form “Mix-3”. 62.42 g of isocyanate component was added to Mix-3 and the ingredients were mixed for 60±10 seconds to form “System A”.

3.39 g of distilled water was mixed with 0.53 g of blowing catalyst in a small plastic cup for 60 seconds with a glass rod to form “System B”.

System B was poured into System A as quickly as possible while avoiding spillage. The ingredients were mixed vigorously with the drill mixer as described above for 10 seconds then poured into a 22.9 cm×20.3 cm×12.7 cm (9 in.×8 in.×5 in.) cardboard box with its inside surfaces covered by aluminum foil. The foaming profile was as follows: 11 seconds mixing time, 27 seconds cream time, and 100 seconds rise time.

2 minutes after the beginning of foaming, i.e., the time when Systems A and B were combined, the foam was place into a circulating-air oven maintained at 100-105° C. for curing for from about 55 to about 60 minutes. Thereafter, the foam was removed from the oven and cooled for 10 minutes at about 25° C. The skin was removed from each side using a band saw. Thereafter, hand pressure was applied to each side of the foam to open the cell windows. The foam was replaced into the circulating-air oven and postcured at 100-105° C. for additional 4.5 hours.

The average pore diameter of the foam, as determined from optical microscopy observations, was greater than about 325 μm.

The following foam testing was carried out according to ASTM D3574. Bulk density was measured using specimens of dimensions 50 mm×50 mm×25 mm. The density was calculated by dividing the weight of the sample by the volume of the specimen. A density value of 2.29 lbs/ft³ (0.037 g/cc) was obtained.

Tensile tests were conducted on samples that were cut either parallel to or perpendicular to the direction of foam rise. The dog-bone shaped tensile specimens were cut from blocks of foam. Each test specimen measured about 12.5 mm thick, about 25.4 mm wide and about 140 mm long; the gage length of each specimen was 35 mm and the gage width of each specimen was 6.5 mm. Tensile properties (tensile strength and elongation at break) were measured using an INSTRON Universal Testing Instrument Model 1122 with a cross-head speed of 500 mm/min (19.6 inches/minute). The average tensile strength parallel to the direction of foam rise was determined as about 33.8 psi (23,770 kg/m²). The elongation to break parallel to the direction of foam rise was determined to be about 123%. The average tensile strength perpendicular to the direction of foam rise was determined as about 27.2 psi (19,150 kg/m²). The elongation to break perpendicular to the direction of foam rise was determined to be about 134%.

### Example 2

### Reticulation of Cross-Linked Polyurethane Matrix 1 and Fabrication of Implantable Devices Therefrom

Reticulation of the foam described in Example 1 was carried out by the procedure described in Example 6.

The density of the reticulated foam was determined as described in Example 1. A post-reticulation density value of 2.13 lbs/ft³ (0.034 g/cc) was obtained.

Tensile tests were conducted on reticulated foam samples as described in Example 1. The average post-reticulation tensile strength parallel to the direction of foam rise was determined as about 31.1 psi (21,870 kg/m²). The post-reticulation elongation to break parallel to the direction of foam rise was determined to be about 92%. The average post-reticulation tensile strength perpendicular to the direction of foam rise was determined as about 22.0 psi (15,480 kg/m²). The post-reticulation elongation to break perpendicular to the direction of foam rise was determined to be about 110%.

Compressive tests were conducted using specimens measuring 50 mm×50 mm×25 mm. The tests were conducted using an INSTRON Universal Testing Instrument Model 1122 with a cross-head speed of 10 mm/min (0.4 inches/minute). The post-reticulation compressive strengths, at 50% and 75% compression, each parallel to the direction of foam rise were determined to be 1.49 psi (1,050 kg/m²) and 3.49 psi (2,460 kg/m²), respectively. The post-reticulation compressive sets, parallel to the direction of foam rise, at 50% and 75% compression, each determined after subjecting the reticulated sample to the stated amount of compression for 22 hours at 25° C. then releasing the compressive stress, were determined to be about 4.7% and 7.5%, respectively.

Mushroom-shaped implantable devices, with a flat cylindrical head or cap of about 16 mm in diameter and about 8 mm in length, and a narrow cylindrical stem of about 10 mm diameter and about 8 mm in length, were machined from the reticulated foam. Thereafter, the samples were sterilized by exposing them to a gamma radiation dose of about 2.3 Mrad.

### Example 3

### Fabrication of Collagen-Coated Implantable Devices

Type I collagen, obtained by extraction from a bovine source, was washed and chopped into fibrils. A 1% by weight collagen aqueous slurry was made by vigorously stirring the collagen and water and adding inorganic acid to a pH of about 3.5. The viscosity of the slurry was about 500 centipoise.

The mushroom-shaped implantable devices prepared according to Example 2 were completely immersed in the collagen slurry, thereby impregnating each implantable device with the slurry. Thereafter, the collagen-slurry impregnated devices were placed on metal trays which were placed onto a lyophilizer shelf pre-cooled to −45° C. After the slurry in the devices froze, the pressure within the lyophilization chamber was reduced to about 100 millitorr, thereby subliming the water out of the frozen collagen slurry leaving a porous collagen matrix deposited within the pores of the reticulated implantable devices. Thereafter, the temperature was slowly raised to about 25° C., then the pressure was returned to 1 atmosphere. The total treatment time in the lyophilizer was about 21-22 hours.

After the implantable devices were removed from the lyophilizer, the collagen was cross-linked by placing the dry collagen impregnated implants in contact with formaldehyde vapor for about 21 hours. Thereafter, the samples were sterilized by exposing them to a gamma radiation dose of about 2.3 Mrad.

### Example 4

### Implantation of Implants into Pig L1 through L4 Lumbar Spaces

Yucatan mini pigs weighing about 55-65 kg each underwent L1 through L4 (lumbar spaces) discectomy. The discectomy consisted of a posteriorlateral annulotomy and nuclectomy paralleling the accepted human clinical surgical procedure. The mushroom-shaped implantable devices made by the procedures described in Examples 2 and 3 were implanted in a 3 mm anterior lateral annulotomy to repair the annular defect. Standard closure procedure was followed. Each of the implantable devices of the invention functioned well, e.g., it conformally expanded, obliterated the annular defect, and maintained its position. There were no adverse acute events associated with the procedure and all subject animals recovered uneventfully.

### Example 5

### Synthesis and Properties of Reticulated Elastomeric Matrix 1

A reticulated cross-linked biodurable elastomeric polycarbonate urea-urethane matrix was made by the following procedure.

The aromatic isocyanate MONDUR MRS-20 (from Bayer Corporation) was used as the isocyanate component. MONDUR MRS-20 is a liquid at 25° C. MONDUR MRS-20 contains 4,4′-diphenylmethane diisocyanate (MDI) and 2,4′-MDI and has an isocyanate functionality of about 2.2 to 2.3. A diol, poly(1,6-hexanecarbonate) diol (POLY-CD220 from Arch Chemicals) with a molecular weight of about 2,000 Daltons, was used as the polyol component and was a solid at 25° C. Distilled water was used as the blowing agent. The catalysts used were the amines triethylene diamine (33% by weight in dipropylene glycol; DABCO 33LV from Air Products) and bis(2-dimethylaminoethyl)ether (23% by weight in dipropylene glycol; NIAX A-133 from GE Silicones). Silicone-based surfactants TEGOSTAB BF 2370 and TEGOSTAB B-8305 (from Goldschmidt) were used for cell stabilization. A cell-opener was used (ORTEGOL 501 from Goldschmidt). The viscosity modifier propylene carbonate (from Sigma-Aldrich) was present to reduce the viscosity. Glycerine (99.7% USP Grade) and 1,4-butanediol (99.75% by weight purity, from Lyondell) were added to the mixture as, respectively, a cross-linking agent and a chain extender. The proportions of the ingredients that were used is given in Table 3 below.

TABLE 3

| Ingredient | Parts by Weight |
|---|---|
| Polyol Component | 100 |
| Isocyanate Component | 52.96 |
| Isocyanate Index | 1.00 |
| Viscosity Modifier | 5.80 |
| Cell Opener | 2.00 |
| Distilled Water | 1.95 |
| B-8305 Surfactant | 0.70 |
| BF 2370 Surfactant | 0.70 |
| 33LV Catalyst | 0.45 |
| A-133 Catalyst | 0.12 |
| Glycerine | 2.00 |
| 1,4-Butanediol | 0.80 |

The isocyanate index, a quantity well known in the art, is the mole ratio of the number of isocyanate groups in a formulation available for reaction to the number of groups in the formulation that are able to react with those isocyanate groups, e.g., the reactive groups of diol(s), polyol component(s), chain extender(s), water and the like, when present. The isocyanate component of the formulation was placed into the component A metering system of an Edge Sweets Bench Top model urethane mixing apparatus and maintained at a temperature of about 20-25° C.

The polyol was liquefied at about 70° C. in an oven and combined with the viscosity modifier and cell opener in the aforementioned proportions to make a homogeneous mixture. This mixture was placed into the component B metering system of the Edge Sweets apparatus. This polyol component was maintained in the component B system at a temperature of about 65-70° C.

The remaining ingredients from Table 3 were mixed in the aforementioned proportions into a single homogeneous batch and placed into the component C metering system of the Edge Sweets apparatus. This component was maintained at a temperature of about 20-25° C. During foam formation, the ratio of the flow rates, in grams per minute, from the supplies for component A:component B:component C was about 8:16:1.

The above components were combined in a continuous manner in the 250 cc mixing chamber of the Edge Sweets apparatus that was fitted with a 10 mm diameter nozzle placed below the mixing chamber. Mixing was promoted by a high-shear pin-style mixer operating in the mixing chamber. The mixed components exited the nozzle into a rectangular cross-section release-paper coated mold. Thereafter, the foam rose to substantially fill the mold. The resulting mixture began creaming about 10 seconds after contacting the mold and was at full rise within 120 seconds. The top of the resulting foam was trimmed off and the foam was placed into a 100° C. curing oven for 5 hours.

Following curing, the sides and bottom of the foam block were trimmed off then the foam was placed into a reticulator device comprising a pressure chamber, the interior of which was isolated from the surrounding atmosphere. The pressure in the chamber was reduced so as to remove substantially all the air in the cured foam. A mixture of hydrogen and oxygen gas, present at a ratio sufficient to support combustion, was charged into the chamber. The pressure in the chamber was maintained above atmospheric pressure for a sufficient time to ensure gas penetration into the foam. The gas in the chamber was then ignited by a spark plug and the ignition exploded the gas mixture within the foam. To minimize contact with any combustion products and to cool the foam, the resulting combustion gases were removed from the chamber and replaced with about 25° C. nitrogen immediately after the explosion. Then, the above-described reticulation process was repeated one more time. Without being bound by any particular theory, the explosions were believed to have at least partially removed many of the cell walls or “windows” between adjoining cells in the foam, thereby creating open pores and leading to a reticulated elastomeric matrix structure.

The average cell diameter or other largest transverse dimension of Reticulated Elastomeric Matrix 1, as determined from optical microscopy observations, was about 525 μm. FIG. 10 is a scanning electron micrograph (SEM) image of Reticulated Elastomeric Matrix 1 demonstrating, e.g., the network of cells interconnected via the open pores therein and the communication and interconnectivity thereof. The scale bar at the bottom edge of FIG. 10 corresponds to about 500 μm. The average pore diameter or other largest transverse dimension of Reticulated Elastomeric Matrix 1, as determined from SEM observations, was about 205 μm.

The following tests were carried out on the thus-formed Reticulated Elastomeric Matrix 1, obtained from reticulating the foam, using test methods based on ASTM Standard D3574. Bulk density was measured using Reticulated Elastomeric Matrix 1 specimens of dimensions 5.0 cm×5.0 cm×2.5 cm. The post-reticulation density was calculated by dividing the weight of the specimen by the volume of the specimen. A density value of 3.29 lbs/ft³ (0.053 g/cc) was obtained.

Tensile tests were conducted on Reticulated Elastomeric Matrix 1 specimens that were cut either parallel to or perpendicular to the foam-rise direction. The dog-bone shaped tensile specimens were cut from blocks of reticulated elastomeric matrix. Each test specimen measured about 1.25 cm thick, about 2.54 cm wide, and about 14 cm long. The gage length of each specimen was 3.5 cm and the gage width of each specimen was 6.5 mm. Tensile properties (tensile strength and elongation at break) were measured using an INSTRON Universal Testing Instrument Model 3342 with a cross-head speed of 50 cm/min (19.6 inches/min). The average post-reticulation tensile strength perpendicular to the foam-rise direction was determined to be about 34.3 psi (24,115 kg/m²). The post-reticulation elongation to break perpendicular to the foam-rise direction was determined to be about 124%. The average post-reticulation tensile strength parallel to the foam-rise direction was determined to be about 61.4 psi (43,170 kg/m²). The post-reticulation elongation to break parallel to the foam-rise direction was determined to be about 122%.

Compressive tests were conducted using Reticulated Elastomeric Matrix 1 specimens measuring 5.0 cm×5.0 cm×2.5 cm. The tests were conducted using an INSTRON Universal Testing Instrument Model 1122 with a cross-head speed of 1 cm/min (0.4 inches/min). The post-reticulation compressive strength at 50% compression, parallel to the foam-rise direction, was determined to be about 2.1 psi (1,475 kg/m²). The post-reticulation compression set, determined after subjecting the reticulated specimen to 50% compression for 22 hours at 25° C. then releasing the compressive stress, parallel to the foam-rise direction, was determined to be about 8.5%.

The static recovery of Reticulated Elastomeric Matrix 1 was measured by subjecting cylindrcular specimens, each 12 mm in diameter and 6 mm in thickness, to a 50% uniaxial compression in the foam-rise direction using the standard compressive fixture in a Q800 Dynamic Mechanical Analyzer (TA Instruments, New Castle, Del.) for 120 minutes followed by 120 minutes of recovery time. The time required for recovery to 90% of the specimen's initial thickness of 6 mm (“t-90%”) was measured and the average determined to be 1406 seconds.

The resilient recovery of Reticulated Elastomeric Matrix 1 was measured by subjecting rectangular parallelepiped specimens, each 1 inch (2.54 cm) high (in the foam-rise direction)×1.25 inches×1.25 inches (3.18 cm×3.18 cm), to a 50% uniaxial compression in the foam-rise direction and then, while maintaining that uniaxial compression, imparting, in an air atmosphere, a dynamic loading off 5% strain at a frequency of 1 Hz for 5,000 cycles or 100,000 cycles, also in the foam-rise direction. Additionally, rectangular parallelepiped specimens were also tested as described above for 100,000 cycles except that the samples were submerged in water throughout the testing. The time required for recovery to 67% (“t-67%”) and 90% (“t-90%”) of the specimens' initial height of 1 inch (2.54 cm) was measured and recorded. The results obtained are shown in Table 4.

TABLE 4

| | | Test Specimen | | |
|---|---|---|---|---|
| No. of Cycles at | | Orientation | | |
| 50% Compression ± 5% | | Relative to Foam- | t-67% | t-90% |
| Strain at 1 Hz | | Rise Direction | (sec) | (sec) |
| 5,000 | (in air) | Parallel | 0.7 | 46 |
| 100,000 | (in air) | Parallel | 84 | 2370 |
| 100,000 | (in water) | Parallel | — | 3400 |

Fluid, e.g., liquid, permeability through Reticulated Elastomeric Matrix 1 was measured in the foam-rise direction using an Automated Liquid Permeameter—Model LP-101-A (also from Porous Materials, Inc.). The cylindrical reticulated elastomeric matrix specimens tested were between 7.0-7.7 mm in diameter and 13-14 mm in length. A flat end of a specimen was placed in the center of a metal plate that was placed at the bottom of the Liquid Permeaeter apparatus. To measure liquid permeability, water was allowed to extrude upward, driven by pressure from a fluid reservoir, from the specimen's end through the specimen along its axis. The operations associated with permeability measurements were fully automated and controlled by a Capwin Automated Liquid Permeameter (version 6.71.92) which, together with Microsoft Excel software, performed all the permeability calculations. The permeability of Reticulated Elastomeric Matrix 1 was determined to be 498 Darcy in the foam-rise direction.

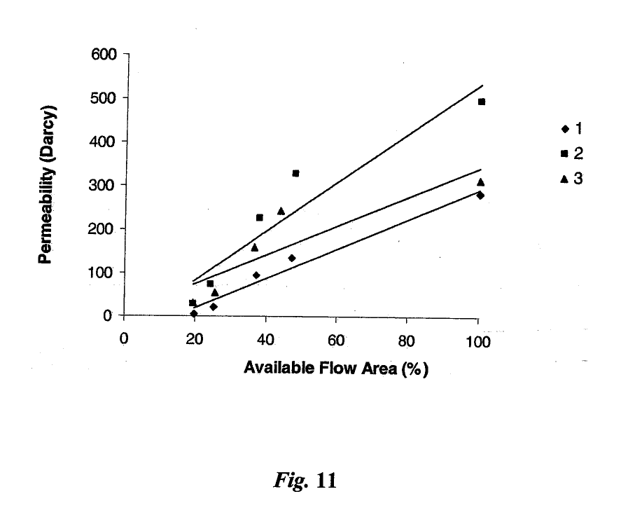

Permeability was also measured after Reticulated Elastomeric Matrix 1 was compressed (perpendicular to the foam-rise direction) so as to reduce the available flow area, thereby simulating compressive molded samples. This was done by inserting a cylindrical sample, with a diameter greater than the diameter of the stainless steel sample holder, into the holder, thereby radially compressing the sample. The uncompressed cylindrical Reticulated Elastomeric Matrix 1 specimens tested were about 7.0 mm in diameter and 13-14 mm in length, while the diameter of the compressed samples ranged from about 9.0 mm to about 16.0 mm prior to their compression into the about 7.0 mm diameter stainless steel holder. FIG. 11 is a plot the Darcy permeability vs. available flow area for reticulated elastomeric matrices of differing formulation; line 2 in FIG. 11 is such a plot for Reticulated Elastomeric Matrix 1. In FIG. 11, 100% Available Flow Area represents uncompressed Reticulated Elastomeric Matrix 1 and demonstrates the highest permeability in the foam-rise direction, 498 Darcy. The change of permeability with available flow area is illustrated by the plots in FIG. 11. For example, the permeability in the foam-rise direction for Reticulated Elastomeric Matrix 1 decreased to 329 Darcy when the available flow area after compression was reduced to 47.9% of the original area and to 28 Darcy when the available flow area after compression was reduced to 19.4% of the original area.

### Example 6

### Synthesis and Properties of Reticulated Elastomeric Matrix 2

A reticulated cross-linked biodurable elastomeric polycarbonate urea-urethane matrix was made by the procedure described in Example 5 except that the ingredients used and their proportions are given in Table 5 below.

TABLE 5

| Ingredient | Parts by Weight |
|---|---|
| Polyol Component | 100 |
| Isocyanate Component | 52.37 |
| Isocyanate Index | 1.00 |
| Viscosity Modifier | 5.80 |
| Cell Opener | 2.00 |
| Distilled Water | 2.15 |
| B-8305 Surfactant | 0.70 |
| BF 2370 Surfactant | 0.72 |
| 33LV Catalyst | 0.55 |
| Glycerine | 2.00 |
| 1,4-Butanediol | 1.95 |

The average cell diameter or other largest transverse dimension of Reticulated Elastomeric Matrix 2, as determined from optical microscopy observations, was about 576 μm. SEM images of Reticulated Elastomeric Matrix 2 demonstrated, e.g., the network of cells interconnected via the open pores therein. The average pore diameter or other largest transverse dimension of Reticulated Elastomeric Matrix 2, as determined from SEM observations, was about 281 μm.

The following tests were carried out on the thus-formed Reticulated Elastomeric Matrix 2, obtained from reticulating the foam, using test methods based on ASTM Standard D3574. The density of Reticulated Elastomeric Matrix 2 was determined as described in Example 5; a density value of 3.23 lbs/ft³ (0.053 g/cc) was obtained.

Tensile tests were conducted on Reticulated Elastomeric Matrix 2 as described in Example 5. The average post-reticulation tensile strength perpendicular to the foam-rise direction was determined to be about 40 psi (28,120 kg/m²). The post-reticulation elongation to break perpendicular to the foam-rise direction was determined to be about 135%. The average post-reticulation tensile strength parallel to the foam-rise direction was determined to be about 55 psi (38,665 kg/m²). The post-reticulation elongation to break parallel to the foam-rise direction was determined to be about 126%.

Compressive tests were conducted using Reticulated Elastomeric Matrix 2 specimens as described in Example 5. The post-reticulation compressive strength at 50% compression, parallel to the foam-rise direction, was determined to be about 2.0 psi (1,406 kg/m²). The post-reticulation compression set, determined after subjecting the reticulated specimen to 50% compression for 22 hours at 25° C. then releasing the compressive stress, parallel to the foam-rise direction, was determined to be about 7.5%.

The resilient recovery of Reticulated Elastomeric Matrix 2 was measured as described in Example 5. The results obtained are shown in Table 6.

TABLE 6

| | | Test Specimen | | |
|---|---|---|---|---|
| No. of Cycles at | | Orientation | | |
| 50% Compression ± 5% | | Relative to Foam- | t-67% | t-90% |
| Strain at 1 Hz | | Rise Direction | (sec) | (sec) |
| 5,000 | (in air) | Parallel | — | 123 |
| 100,000 | (in air) | Parallel | 50 | 3845 |
| 100,000 | (in water) | Parallel | — | 2350 |

Fluid permeability through Reticulated Elastomeric Matrix 2 was measured in the foam-rise direction as described in Example 5 using the Automated Liquid Permeameter, Model LP-101-A. The permeability of Reticulated Elastomeric Matrix 2 was determined to be 314 Darcy in the foam-rise direction.

Permeability was also measured after Reticulated Elastomeric Matrix 2 was compressed (perpendicular to the foam-rise direction) so as to reduce the available flow area, as described in Example 5. Line 3 in FIG. 11 is a plot of the Darcy permeability vs. available flow area for Reticulated Elastomeric Matrix 2. In FIG. 11, the 100% Available Flow Area represents uncompressed Reticulated Elastomeric Matrix 2 and demonstrates the highest permeability in the foam-rise direction, 314 Darcy. The permeability in the foam-rise direction for Reticulated Elastomeric Matrix 2 decreased to 224 Darcy when the available flow area after compression was reduced to 43.9% of the original area and to 54 Darcy when the available flow area after compression was reduced to 25.5% of the original area.

### Example 7

### Synthesis and Properties of Reticulated Elastomeric Matrix 3

A reticulated cross-linked biodurable elastomeric polycarbonate urea-urethane matrix was made by the procedure described in Example 5 except that the ingredients used and their proportions are given in Table 7 below.

TABLE 7

| Ingredient | Parts by Weight |
|---|---|
| Polyol Component | 100 |
| Isocyanate Component | 46.90 |
| Isocyanate Index | 1.00 |
| Viscosity Modifier | 5.80 |
| Cell Opener | 2.00 |
| Distilled Water | 1.00 |
| B-8305 Surfactant | 1.00 |
| BF 2370 Surfactant | 1.00 |
| 33LV Catalyst | 0.45 |
| A-133 Catalyst | 0.15 |
| Glycerine | 3.00 |
| 1,4-Butanediol | 2.00 |

The average cell diameter or other largest transverse dimension of Reticulated Elastomeric Matrix 3, as determined from optical microscopy observations, was about 300 μm. FIG. 12 is a SEM image of Reticulated Elastomeric Matrix 3 demonstrating, e.g., the network of cells interconnected via the open pores therein and the communication and interconnectivity thereof. The average pore diameter or other largest transverse dimension of Reticulated Elastomeric Matrix 3, as determined from SEM observations, was about 175 μm.

The following tests were carried out on the thus-formed Reticulated Elastomeric Matrix 3, obtained from reticulating the foam, using test methods based on ASTM Standard D3574. The density of Reticulated Elastomeric Matrix 3 was determined as described in Example 5; a density value of 5.92 lbs/ft³ (0.095 g/cc) was obtained.

Tensile tests were conducted on Reticulated Elastomeric Matrix 3 specimens as described in Example 5. The average post-reticulation tensile strength perpendicular to the foam-rise direction was determined to be about 71.7 psi (50,405 kg/m²). The post-reticulation elongation to break perpendicular to the foam-rise direction was determined to be about 161%. The average post-reticulation tensile strength parallel to the foam-rise direction was determined to be about 104 psi (73,110 kg/m²). The post-reticulation elongation to break parallel to the foam-rise direction was determined to be about 169%.

Compressive tests were conducted using Reticulated Elastomeric Matrix 3 specimens as described in Example 5. The post-reticulation compressive strength at 50% compression, parallel to the foam-rise direction, was determined to be about 3.65 psi (2,565 kg/m²).

The static recovery of Reticulated Elastomeric Matrix 3 specimens was measured as described in Example 5. T-90% was measured and the average determined to be 166 seconds.

The resilient recovery of Reticulated Elastomeric Matrix 3 was measured as described in Example 5. The results obtained are shown in Table 8.

TABLE 8

| | | Test Specimen | | |
|---|---|---|---|---|
| No. of Cycles at | | Orientation | | |
| 50% Compression ± 5% | | Relative to Foam- | t-67% | t-90% |
| Strain at 1 Hz | | Rise Direction | (sec) | (sec) |
| 5,000 | (in air) | Parallel | — | 13.6 |
| 100,000 | (in air) | Parallel | — | 175 |
| 100,000 | (in water) | Parallel | — | 108 |

Fluid permeability through Reticulated Elastomeric Matrix 3 was measured in the foam-rise direction as described in Example 5 using the Automated Liquid Permeameter, Model LP-101-A. The permeability of Reticulated Elastomeric Matrix 3 was determined to be 103 Darcy in the foam-rise direction.

### Example 8

### Synthesis and Properties of Reticulated Elastomeric Matrix 4

A reticulated cross-linked biodurable elastomeric polycarbonate urea-urethane matrix was made by the procedure described in Example 5 except that the ingredients used and their proportions are given in Table 9 below.

TABLE 9

| Ingredient | Parts by Weight |
|---|---|
| Polyol Component | 100 |
| Isocyanate Component | 45.64 |
| Isocyanate Index | 1.00 |
| Viscosity Modifier | 5.80 |
| Cell Opener | 2.00 |
| Distilled Water | 1.60 |
| B-8305 Surfactant | 1.00 |
| BF 2370 Surfactant | 1.00 |
| 33LV Catalyst | 0.45 |
| A-133 Catalyst | 0.15 |
| Glycerine | 1.00 |
| 1,4-Butanediol | 1.50 |

The average cell diameter or other largest transverse dimension of Reticulated Elastomeric Matrix 4, as determined from optical microscopy observations, was about 353 μm. SEM images of the reticulated elastomeric matrix of this example demonstrated, e.g., the network of cells interconnected via the open pores therein. The average pore diameter or other largest transverse dimension of Reticulated Elastomeric Matrix 4, as determined from SEM observations, was about 231 μm.

The following tests were carried out on the thus-formed Reticulated Elastomeric Matrix 4, obtained from reticulating the foam, using test methods based on ASTM Standard D3574. The density of Reticulated Elastomeric Matrix 4 was determined as described in Example 5; a density value of 3.81 lbs/ft³ (0.061 g/cc) was obtained.

Tensile tests were conducted on Reticulated Elastomeric Matrix 4 specimens as described in Example 5. The average post-reticulation tensile strength perpendicular to the foam-rise direction was determined to be about 40.9 psi (28,753 kg/m²). The post-reticulation elongation to break perpendicular to the foam-rise direction was determined to be about 216%. The average post-reticulation tensile strength parallel to the foam-rise direction was determined to be about 52.5 psi (36,910 kg/m²). The post-reticulation elongation to break parallel to the foam-rise direction was determined to be about 206%.

Compressive tests were conducted using Reticulated Elastomeric Matrix 4 specimens as described in Example 5. The post-reticulation compressive strength at 50% compression, parallel to the foam-rise direction, was determined to be about 1.3 psi (914 kg/m²).

The static recovery of Reticulated Elastomeric Matrix 4 specimens was measured as described in Example 5. T-90% was measured and the average determined to be 466 seconds.

The resilient recovery of Reticulated Elastomeric Matrix 4 was measured as described in Example 5. The results obtained are shown in Table 10.

TABLE 10

| | | Test Specimen | | |
|---|---|---|---|---|
| No. of Cycles at | | Orientation | | |
| 50% Compression ± 5% | | Relative to Foam- | t-67% | t-90% |
| Strain at 1 Hz | | Rise Direction | (sec) | (sec) |
| 5,000 | (in air) | Parallel | 0.6 | 7.0 |
| 100,000 | (in air) | Parallel | 3.0 | 761 |
| 100,000 | (in water) | Parallel | — | 382 |

Fluid permeability through Reticulated Elastomeric Matrix 4 was measured in the foam-rise direction as described in Example 5 using the Automated Liquid Permeameter, Model LP-101-A. The permeability of Reticulated Elastomeric Matrix 4 was determined to be 380 Darcy in the foam-rise direction.

### Example 9

### Implantable Device with Selectively Non-Porous Surface

A piece of reticulated material made according to Example 5 is used. A heated blade with a knife-edge is used to cut a cylinder 10 mm in diameter and 15 mm in length from the piece. The blade temperature is above 170° C. The surfaces of the piece in contact with the heated blade appear to be fused and non-porous from contact with the heated blade. Those surfaces of the piece that are intended to remain porous, i.e., not to fuse, are not exposed to the heated blade.

### Example 10

### Implantable Device with Selectively Non-Porous Surface

A slightly oversized piece of reticulated material made according to Example 5 is used. The slightly oversized piece is placed into a mold heated to a temperature of above 170° C. The mold is then closed over the piece to reduce the overall dimensions to the desired size. Upon removing the piece from the mold, the surfaces of the piece in contact with the mold appear to be fused and non-porous from contact with the mold. Those surfaces of the piece that are intended to remain porous, i.e., not to fuse, are protected from exposure to the heated mold. A heated blade with a knife-edge is used to cut from the piece a cylinder 10 mm in diameter and 15 mm length.

### Example 11

### Dip-Coated Implantable Device with Selectively Non-Porous Surface

A piece of reticulated material made according to Example 5 is used. A coating of copolymer containing 90 mole % PGA and 10 mole % PLA is applied to the macro surface as follows. The PGA/PLA copolymer is melted in an extruder at 205° C. and the piece is dipped into the melt to coat it. Those surfaces of the piece that are to remain porous, i.e., not to be coated by the melt, are covered to protect them and not exposed to the melt. Upon removal, the melt solidifies and forms a thin non-porous coating layer on the surfaces of the piece with which it comes in contact.

### Example 12

### Fabrication of a Collagen-Coated Elastomeric Matrix

Type I collagen, obtained by extraction from bovine hide, is washed and chopped into fibrils. A 1% by weight collagen aqueous slurry is made by vigorously stirring the collagen and water and adding inorganic acid to a pH of about 3.5.

A reticulated polyurethane matrix prepared according to Example 5 is cut into a piece measuring 60 mm by 60 mm by 2 mm. The piece is placed in a shallow tray and the collagen slurry is poured over it so that the piece is completely immersed in the slurry for about 15 minutes, and the tray is optionally shaken. If necessary, excess slurry is decanted from the piece and the slurry-impregnated piece is placed on a plastic tray, which is placed on a lyophilizer tray held at 10° C. The lyophilizer tray temperature is dropped from 10° C. to −35° C. at a cooling rate of about 1° C./minute and the pressure within the lyophilizer is reduced to about 75 millitorr. After holding at −35° C. for 8 hours, the temperature of the tray is raised at a rate of about 1° C./hour to 10° C. and then at a rate of about 2.5° C./hour until a temperature of 25° C. is reached. During lyophilization, the water sublimes out of the frozen collagen slurry leaving a porous collagen matrix deposited within the pores of the reticulated polyurethane matrix piece. The pressure is returned to 1 atmosphere.

Optionally, the porous collagen-coated polyurethane matrix piece is subjected to further heat treatment at about 110° C. for about 24 hours in a current of nitrogen gas to cross-link the collagen, thereby providing additional structural integrity.

### Example 13

### Synthesis and Properties of Reticulated Elastomeric Matrix 5 and its Use in an Implantable Device for Repair of the Rat Abdominal Wall

A reticulated cross-linked biodurable elastomeric polycarbonate urea-urethane matrix was made by the following procedure.

The aromatic isocyanate MONDUR MRS 20 (from Bayer; comprising a mixture of 4,4′-MDI and 2,4′-MDI) was used as the isocyanate component. MONDUR MRS 20 contains from about 65% to 70% by weight 4,4′-MDI, from about 30% to 35% by weight 2,4′-MDI, has an isocyanate functionality of about 2.2 to 2.3, and is a liquid at 25° C. A diol, poly(1,6-hexanecarbonate) diol (POLY-CD CD220, Arch Chemicals) with a molecular weight of about 2,000 Daltons was used as the polyol component and was a solid at 25° C. Distilled water was used as the blowing agent. The blowing catalyst was the tertiary amine triethylene diamine (33% by weight in dipropylene glycol; DABCO 33LV from Air Products). Glycerine (99.7% USP/EP, from Dow Chemical) was used as a cross-linking agent and 1,4-butanediol (from BASF Chemical) was used as a chain extender. A silicone-based surfactant was used (TEGOSTAB BF 2370, from Goldschmidt). A cell-opener was used (ORTEGOL 501, from Goldschmidt). The viscosity modifier propylene carbonate (from Sigma-Aldrich) was present to reduce the viscosity. The proportions of the ingredients that were used is given in Table 11 below.

TABLE 11

| Ingredient | Parts by Weight |
|---|---|
| Polyol Component | 100 |
| Isocyanate Component | 51.32 |
| Isocyanate Index | 1.00 |
| Viscosity Modifier | 5.80 |
| Cell Opener | 2.0 |
| Surfactant | 1.5 |
| Distilled Water | 1.89 |
| Blowing Catalyst | 0.56 |
| Glycerine | 2.15 |
| 1,4-Butanediol | 0.72 |

The diol was liquefied at 70° C. in an air-circulation oven, and 100 g of it was weighed into a polyethylene cup. 5.8 g of viscosity modifier (propylene carbonate) was added to the polyol and mixed with a drill mixer equipped with a mixing shaft at 3100 rpm for 15 seconds (mix-1). 1.5 g of surfactant (TEGOSTAB BF-2370) was added to mix-1 and mixed for additional 15 seconds (mix-2). 2.0 g of cell opener (ORTEGOL 501) was added to mix-2 and mixed for 15 seconds (mix-3). 2.15 g of cross-linker (glycerine) was added to mix-3 and mixed for 15 seconds (mix-4). 0.72 g of chain extender (1,4-butanediol) was added to mix-4 and mixed for 15 seconds (mix-5). 51.32 g of isocyanate (MONDUR MRS 20) was added to mix-5 and mixed for 60 seconds (system A). 1.89 g of distilled water was mixed with 0.56 g of blowing catalyst (DABCO 33LV) in a small plastic cup by using a small glass rod for 60 seconds (System B).

System B was poured into System A as quickly as possible without spilling and with vigorous mixing with a drill mixer for 10 seconds and poured into a cardboard box of dimensions 9 in.×8 in.×5 in. (23 cm×20 cm×13 cm), which was covered inside with aluminum foil. The foaming profile was as follows: mixing time of 10-12 sec, cream time of 28 sec, and rise time of 120 sec.

Two minutes after beginning of foam mixing, the foam was placed in the oven at 100° C. to 105° C. for curing for 60 minutes. The elastomeric matrix was taken from the oven and cooled for 10 minutes at about 25° C. The skin was removed with a saw and the elastomeric matrix was pressed by hand from all sides to open the cell windows. The elastomeric matrix was put back into the air-circulation oven for postcuring at 100° C. to 105° C. for additional 3.5 hours. Both physical and chemical cross-links were present in the final elastomeric matrix.

Following curing, the sides and bottom of the foam block were trimmed off then the elastomeric matrix was reticulated as described in Example 5. The average pore diameter or other largest transverse dimension of Reticulated Elastomeric Matrix 5, as determined by optical microscopy observations, was about 220 μm.

The following tests were carried out on the thus-formed Reticulated Elastomeric Matrix 5, obtained from reticulating the foam, using test methods based on ASTM Standard D3574. The density of Reticulated Elastomeric Matrix 5 was determined as described in Example 5; a density value of 4.27 lbs/ft³ (0.068 g/cc) was obtained.

Tensile tests were conducted on Reticulated Elastomeric Matrix 5 specimens as described in Example 5. The average post-reticulation tensile strength perpendicular to the foam-rise direction was determined to be about 36.8 psi (25,870 kg/m²). The post-reticulation elongation to break perpendicular to the foam-rise direction was determined to be about 114%. The average post-reticulation tensile strength parallel to the foam-rise direction was determined to be about 66.6 psi (46,805 kg/m²). The post-reticulation elongation to break parallel to the foam-rise direction was determined to be about 117%.

Tear resistance strength of the Reticulated Elastomeric Matrix 5 was measured with specimens measuring approximately 152 mm in length, 25 mm in width and 12.7 mm in height pursuant to the test method described in ASTM Standard D3574. A 40 mm cut was made on one side of each specimen. The tear strength was measured using an INSTRON Universal Testing Instrument Model 1122 with a cross-head speed of 50 cm/min (19.6 inches/min). The tear strength was determined to be about 3.15 lbs/linear inch (526 g/linear cm).

An example of an implantable device according to the invention, a square patch measuring 1 cm in length and width×2 mm in height, was made using Reticulated Elastomeric Matrix 5 and incorporating a 4-0 multifilament polyester fiber (Telflex Medical) therein. The braided polyester fiber (with a diameter equivalent to a 4-0 suture having a maximum diameter of 0.20 mm and a minimum tensile strength of 1.65 lbs (748 g)) was incorporated into the square implantable device using a Viking Platinum Model 730 sewing machine with stitch type 1 and a pitch of 3 mm.

An implantable device was placed in the abdominal wall of a Sprague-Dawley rat. The abdominal wall defect was of partial thickness and left the abdominal fascia and the peritoneum and skin intact. Stated differently, the internal and external abdominal oblique muscles were excised and replaced by the test implantable device in the rat. Therefore, there was no device entry into the abdominal cavity and the skin was intact following surgical closure of the operative site. The device was surrounded by native muscle tissue, subcutaneous tissue and fascia. The rat was sacrificed at 16 weeks after implantation.

Histology analysis at 16 weeks showed tissue ingrowth and proliferation throughout the implanted device. The implanted device promoted repair of the abdominal wall defect in the rat. The device demonstrated favorable response and was well bio-integrated with good tissue in-growth.

### Example 14

### Manufacture of an Implantable Device from Reticulated Elastomeric Matrix 4 and Braided Fiber Reinforcement

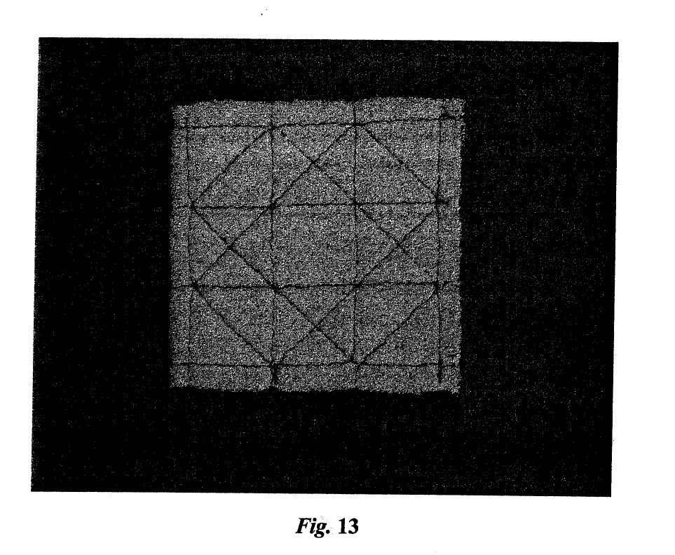

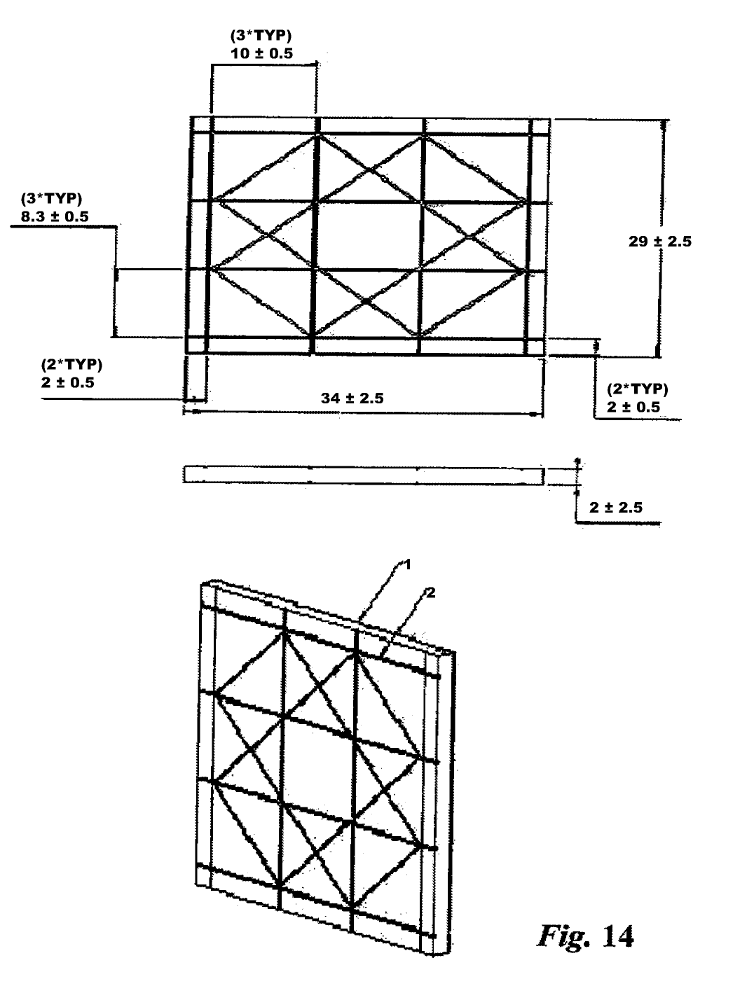

Reticulated Elastomeric Matrix 4 was made by following procedures described in Example 8. An implantable device, such as a surgical patch, shaped as a rectangular patch having dimensions of 29 mm in length, 34 mm in width and 2 mm in thickness, was cut from the reticulated elastomeric matrix. Braided polyester fibers (Telflex Medical; diameter equivalent to a 5-0 suture and having a maximum diameter of 0.15 mm and a minimum tensile strength of 0.88 lbs (399 g)) were incorporated into the rectangular implantable device using an embroidery machine (Baby Lock Esante model BLN) with the pattern illustrated in FIG. 13. The dimensions for features of the pattern are provided in FIG. 14.

The braided polyester fibers were incorporated into the rectangular implantable device using a cross stitch with the following settings: line sew run pitch=1.5 mm; region sew density=3.9 line/mm; machine tension setup=1.4. The grid dimensions were 10 mm×8 mm with 2 mm borders along each of the four sides.

Each implantable device, incorporating the braided fibers, was tested for suture retention strength (SRS), which is defined as the maximum force required to pull a standard suture through the device, thereby causing it to fail. Each device, incorporating the braided fibers, was also tested for the tensile break strength (TBS), which is defined as the maximum force required for tensile failure for the entire device. Both tests were carried out using a using an INSTRON Universal Testing Instrument Model 3342.

In SRS testing, a 2-0 ETHIBOND braided polyester suture was inserted into one end of the implantable device by using a needle and the suture was attached to the device from 2 mm to 3 mm below the first horizontal grid line and about at the device's center line. A loop, about 50 mm to 60 mm in length, was formed by the two ends of the suture strands. The free end (that was not attached to the suture) of the device was mounted within the flat rubber-coated faces of the bottom fixed jaw and clamped. The SRS test was run under displacement mode at a cross-head speed of 100 mm/min (3.94 in/min) with the movable jaws separating or moving upwards and away from the fixed jaws. An average SRS value of 21 Newtons was obtained from testing these implantable devices incorporating the braided polyester fibers.

In the TBS testing of these implantable devices, one end of the device was mounted between the rubber-coated faces mounted onto the fixed pneumatic grip and the other end of the device was mounted between the rubber-coated faces mounted on the movable pneumatic grip. The test was run under displacement mode at a cross-head speed of 100 mm/min (3.94 in/min) with the movable jaws separating or moving upwards and away from the fixed jaws. An average TBS value of 57 Newtons was obtained.

### Example 15

### Use of an Implantable Device with Reticulated Elastomeric Matrix 4 and Braided Fiber Reinforcement in the Augmentation of a Rat Rotator Cuff

An implantable device with Reticulated Elastomeric Matrix 4 and braided polyester fibers and in the shape of a rectangular patch was made similarly to the process described in Example 14 except that 7-0 braided polyester fibers were used. A small square, in the form of a 2 mm in length and width and 1 mm thick patch, was cut from the device and implanted for healing of the supraspinatus tendon in a rat.

A surgical treatment using traditional tendon repair using sutures through bone was employed but augmented by using the implantable device described in the previous paragraph. A bilateral supraspinatus tendon tear was surgically created in the rat. In the right shoulder of the rat, a full-thickness, complete transsection of the supraspinatus tendon was performed. The device was sutured on top of the tendon and the tendon-patch construct was repaired to bone using two 5-0 PROLENE transosseous sutures. Eight weeks following the surgical repair, the rat was sacrificed and a histology analysis of the tendon repair was conducted.

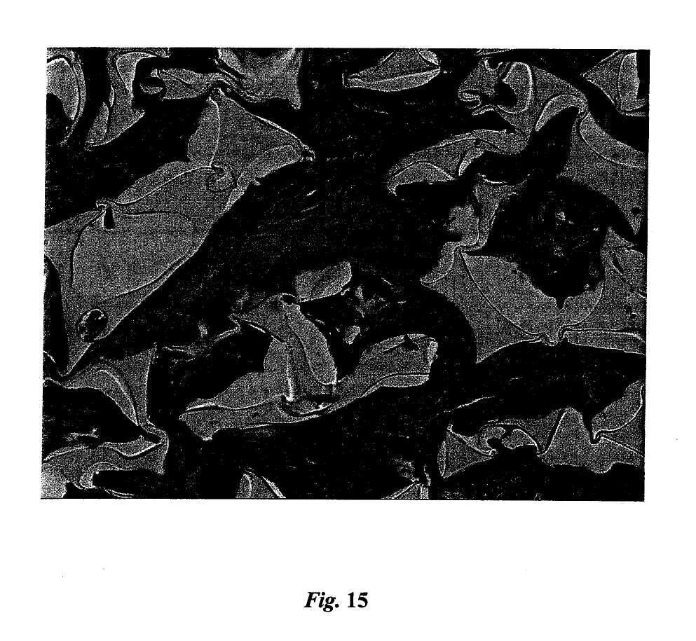

The histology analysis, illustrated by the photograph in FIG. 15, showed no significant amount of inflammation or inappropriate vascularization. The percentage of implantable device void space occupied by tissue ingrowth, determined from analysis of the area occupied by tissue ingrowth in photographs such as FIG. 15, was at least about 80%. For the tissue ingrowth within the implantable device, as visualized by conventional H&E staining, the cellular morphology closest to the device was consistent with connective tissue cells, such as fibroblasts, that are active in collagen matrix production while the cells distal (or further removed from the cells closest to the implantable device) appeared to be more quiescent. The tissue surrounding the implantable device was grossly organized. Tissue areas within the device were organized within any given pore of the reticulated elastomeric matrix comprising the device. However, the tissue within the implantable device was still not fully organized at the time of the sacrifice, as the healing time was probably not sufficiently long.

### Example 16

### Synthesis and Properties of Reticulated Elastomeric Matrix 6 and its Use in an Implantable Device with Braided Fiber Reinforcement for the Repair of a Rat Rotator Cuff

A reticulated cross-linked biodurable elastomeric polycarbonate urea-urethane matrix was made by a process similar to that described in Example 13 except that the aromatic isocyanate RUBINATE 9258 (from Huntsman, comprising a mixture of 4,4′-MDI and 2,4′-MDI), was used as the isocyanate component and no cross-linking agent and chain extender were used. RUBINATE 9258 contains about 68% by weight 4,4′-MDI, about 32% by weight 2,4′-MDI, has an isocyanate functionality of about 2.33, and is a liquid at 25° C. A polyol, 1,6-hexamethylene carbonate (POLY-CD CD220), i.e., a diol, with a molecular weight of about 2,000 Daltons, was used as the polyol component and is a solid at 25° C. The proportions of the ingredients that were used is given in Table 12 below.

TABLE 12

| Ingredient | Parts by Weight |
|---|---|
| Polyol Component | 100 |
| Isocyanate Component | 47.25 |
| Isocyanate Index | 1.00 |
| Viscosity Modifier | 5.80 |
| Cell Opener | 1.45 |
| Surfactant | 0.66 |
| Distilled Water | 2.38 |
| Catalyst | 0.53 |

The foaming profile was as follows: mixing time of 10 sec, cream time of 16 sec, and rise time of 80 sec.

Two minutes after beginning of foam mixing, the elastomeric matrix was placed in the oven at 100° C. to 105° C. for curing for 60 minutes. The elastomeric matrix was taken from the oven and cooled for 10 minutes at about 25° C. The skin was removed with a saw and the elastomeric matrix was pressed by hand from all sides to open the cell windows. The elastomeric matrix was put back into the air-circulation oven for postcuring at 100° C. for additional 4.0 hours.

The foam was reticulated once using a process substantially similar to the reticulation process described in Example 5 to yield Reticulated Elastomeric Matrix 6. The average pore diameter or other largest transverse dimension of Reticulated Elastomeric Matrix 6, as determined from optical microscopy observations, was between 275 μm and 350 μm.

The following tests were carried out on the thus-formed Reticulated Elastomeric Matrix 6, obtained from reticulating the foam, using test methods based on ASTM Standard D3574. The density of Reticulated Elastomeric Matrix 6 was determined as described in Example 5; a density value of 2.99 lbs/ft³ (0.046 g/cc) was obtained.

Tensile tests were conducted on Reticulated Elastomeric Matrix 6 specimens as described in Example 5. The average post-reticulation tensile strength perpendicular to the foam-rise direction was determined to be about 33.6 psi (23,625 kg/m²). The post-reticulation elongation to break perpendicular to the foam-rise direction was determined to be about 220%.

Compressive tests were conducted using Reticulated Elastomeric Matrix 6 specimens as described in Example 5. The post-reticulation compressive strength at 50% compression, parallel to the foam-rise direction, was determined to be about 1.25 psi (878 kg/m²).

For surgical implantation, the matrix was sized and shaped appropriately by cutting a block of Reticulated Elastomeric Matrix 6 which had previously been sterilized by gamma radiation. Sprague-Dawley rats (weighing from about 250 g to about 275 g) were used for this experiment. All rats were anesthetized with an intramuscular injection of Ketamine (100 mg/kg) and Xylazine (5 mg/kg). Thereafter, the upper extremities were shaved, aseptically prepped and draped. Antibiotic prophylaxis was provided for a total of seven days.

The surgical exposure involved 2 cm incisions over the dorsal aspects of the shoulder and scapula bilaterally. In each shoulder, the scapular spine was identified, and the deltoid muscle was split in line with its fibers over a distance of 1 cm. The subacromial bursa was opened but not excised. The supraspinatus tendon was visualized as it passed underneath the coracoacromial arch to its insertion on the greater tuberosity of the proximal humerus.

In a tissue extension group (Group 1), a 2 mm wide area of the supraspinatus tendon was excised bilaterally, beginning 1 mm proximal to the insertion site and extending 2 mm further proximally, resulting in a 2 mm by 2 mm defect. This represented approximately 50% of the supraspinatus tendon width, corresponding to a large full thickness rotator cuff tear in humans.

The defect was bridged with a 2 mm by 2 mm and 1 mm thick Reticulated Elastomeric Matrix 6 implantable device of this example, which was interposed between the edge of the tendon and the insertion site on the greater tuberosity. The device was secured distally to the greater tuberosity through transosseous tunnels with two 5-0 PROLENE (Ethicon Inc.) interrupted sutures. The proximal edge of the device was then attached to the lateral edge of the tendon with two 5-0 PROLENE sutures. The deltoid muscle was then re-approximated to the shoulder with interrupted 4-0 VICRYL (Ethicon Inc.) suture, and the skin was closed with 3-0 MONOCRYL (Ethicon Inc.).

In a tissue augmentation group (Group 2), bilateral full thickness defects were created 1 mm proximal to the supraspinatus tendon insertion with a #15 scalpel blade, but in contrast to Group 1, no section of tendon was removed. The defect was then repaired to the insertion site on the greater tuberosity with two 5-0 PROLENE sutures through transosseous tunnels. The repair was additionally reinforced by over-sewing with a reticulated elastomeric matrix implantable device of this example, creating a layered construct consisting of reticulated elastomeric matrix and tendon. The deltoid muscle was then re-approximated to the shoulder with interrupted 4-0 VICRYL (Ethicon Inc.) suture, and the skin was closed with 3-0 MONOCRYL (Ethicon Inc.).

In the Group 1 and Group 2 experiments, all animals were sacrificed six weeks postoperatively by carbon dioxide inhalation. The rat shoulder was evaluated macroscopically for gross evidence of healing and the supraspinatus tendon and proximal humerus were removed for histology analysis. Gross inspection at the time of retrieval revealed good integration into the tendon and bone, no gross inflammatory changes, and minimal scar tissue. Adhesions were found in the subacromial space and subdeltoid region, consistent with post-surgical changes. Histologically, the shoulders did not demonstrate inflammatory cells or inappropriate vascularization. The collagen fibers were aligned within any given pore compartment of the implanted device and organization was that of regular connective tissue with dense collagen fibers. Generally, it was noted that cells further removed from the device were grossly similar to those directly lining the device, indicating no obvious detrimental influence of the reticulated elastomeric matrix material on cell morphology. Histomorphometric evaluation of the Group 1 specimens showed an average fill ratio of reparative tissue infiltration within the device of 77.6% (standard deviation+/−8.3%).

Analogously to Group 1, implanted devices used for tissue augmentation (Group 2) did not demonstrate inflammatory changes or inappropriate vascularization after the six weeks in vivo implantation. Also, minimal scarring consistent with post-surgical changes was encountered. Histology analysis of the implanted devices showed substantially identical results to Group 1. Specifically, there were no significant inflammatory changes. It was also noted that the reparative tissue infiltrating the devices was well bio-integrated with the tendon of the supraspinatus and the tendon attaching to the humerus. Histomorphometric analysis demonstrated an average device infiltration of 79.9% (standard deviation+/−7.7%).

### Example 17

### Use of Reticulated Elastomeric Matrix 2 in an Implantable Device with Braided Fiber Reinforcement

Reticulated Elastomeric Matrix 2 was made following the procedures described in Example 6. Implantable devices, shaped as rectangular patches having dimensions of 54 mm in length, 34 mm in width and 2 mm in thickness, were cut from Reticulated Elastomeric Matrix 2. Multi-filament braided polyester fibers (Telflex Medical; filament diameter equivalent to a 4-0 suture having a diameter of 0.20 mm and a minimum tensile strength of 1.65 lbs (748 grams)) were incorporated in the form of a grid into the rectangular patch shaped device using a Viking Platinum 730 sewing machine. The braided polyester fibers were incorporated into the rectangular patch using a cross stitch with the following settings: Type 1 stitch with a pitch of 2.5 mm and a tension of 6.5. The dimensions of the square grid were 10 mm×10 mm with 2 mm borders along each of the four sides.

The SRS and TBS were tested using the same method described in Example 14. The magnitude of the SRS was 36.5 Newtons with an extension of 25 mm recorded at the failure of the implantable device subjected to pulling by the 2-0 ETHIBOND suture. The magnitude of the TBS was 56 Newtons with an extension of 7.1 mm at the tensile failure of the entire device.

### Example 18

### Compressive Molding of Reticulated Elastomeric Matrix 1

Reticulated Elastomeric Matrix 1 was made following the procedures described in Example 5. This matrix was compressive molded in 2-dimensions using the following procedure.

Implantable devices shaped as cylinders (“cylindrical pre-forms”) with a diameter of 60.5 mm and a height of 62.0 mm were cut from Reticulated Elastomeric Matrix 1. The cylindrical pre-forms were machined such that the axes of the cylinders were parallel to the foam-rise direction. The cylindrical pre-forms were dried by heating them in an Air Convection Oven (Blue M Inert Gas Oven Model DCA 336F) at 70° C. for 1.5 hours and stored in a dry environment.

Cylindrical molds (each consisting of an aluminum mold base and cover) of 40.5 mm diameter and 62.0 mm height were used for compressive molding the dried cylindrical pre-forms. A dried cylindrical pre-form was press-fitted (at about 25° C.) into each mold so as to impart a compression ratio of 1.49 times in the radial direction, which was perpendicular to the original foam-rise direction. The ratio of the cross-sectional area before and after compression was 2.2 times. The molds, each containing a compressed reticulated elastomeric matrix cylindrical pre-form within, were held in position with adjustable clamps then placed in the oven. The oven was purged with nitrogen. The molds were heated in a nitrogen atmosphere in the oven for 3.0 hours at a temperature of 130° C. Thereafter, the molds were removed from the oven and cooled for 15 minutes using compressed air before the clamps were loosened. The compressed Reticulated Elastomeric Matrix 1 cylindrical pre-forms retained the size and shape of the mold. These compressive molded cylinders were stored in a dry environment.

Properties of the compressive molded reticulated elastomeric matrices were measured using procedures described in Examples 5 and 6. The properties of the reticulated elastomeric matrix before and after compressive molding are presented in Table 13 below, which demonstrates, e.g., compressive molding's significant enhancement of reticulated elastomeric matrix properties.

TABLE 13

| | Reticulated | Compressive |
|---|---|---|
| | Elastomeric | Molded |
| | Matrix 1 | Reticulated |
| | (No Compressive | Elastomeric |
| Property | Molding) | Matrix 1 |
| Density | 3.17 lbs/ft³ | 7.42 lbs/ft³ |
| | (0.051 g/cc) | (0.119 g/cc) |
| Tensile Strength Parallel to | 52.9 psi | 115.9 psi |
| Foam-Rise Direction | (37,190 kg/m²) | (81,480 kg/m²) |
| Elongation Parallel to | 111% | 95% |
| Foam-Rise Direction | | |
| Tensile Strength Perpendicular | 35.4 psi | 45.9 psi |
| to Foam-Rise Direction | (24,890 kg/m²) | (32,270 kg/m²) |
| Elongation Perpendicular | 112% | 175% |
| to Foam-Rise Direction | | |
| Compressive Strength Parallel to | 2.1 psi | 8.2 psi |
| Foam-Rise Direction at 50% Strain | (1,475 kg/m²) | (5,765 kg/m²) |
| Permeability (Darcy) | 498 | About 100 |

### Example 19

### Compressive Molded Reticulated Elastomeric Matrix 1 and its Use in an Implantable Device for Repair of the Rat Abdominal Wall

An example of an implantable device according to the invention, a square patch measuring 1 cm in length and width and 2 mm in height, was made using the compressive molded Reticulated Elastomeric Matrix 1 prepared as descried in Example 18 and incorporating a 5-0 multifilament CP Fiber wire (C. P. Medical) therein. The braided fiber was incorporated into the rectangular device using a Viking Platinum Model 730 sewing machine with stitch type 1 and a pitch of 3 mm.

An implantable device was placed in the abdominal wall of each of twenty Sprague-Dawley rats. The abdominal wall defect was of partial thickness and left the abdominal fascia and the peritoneum and skin intact. Stated differently, the internal and external abdominal oblique muscles were excised and replaced by the test implantable device in the rat. Therefore, there was no device entry into the abdominal cavity and the skin was intact following surgical closure of the operative site. The implanted device was surrounded by native muscle tissue, subcutaneous tissue and fascia. Four rats were sacrificed at each of 1, 2, 4, 8 or 16 weeks after implantation.

Also implanted in the above-described abdominal wall defect of each of twenty different Sprague-Dawley rats was a square patch measuring 1 cm in length and width and 2 mm in height that was made as described above using the compressive molded Reticulated Elastomeric Matrix 1 but without incorporating the 5-0 multifilament CP Fiber wire. Four of these rats were also sacrificed at each of 1, 2, 4, 8 or 16 weeks after implantation. These rats were also sacrificed at 1, 2, 4, 8 or 16 weeks after implantation.

At the designated time of sacrifice, the operative site plus surrounding native tissue was explanted and evaluated by histology analysis for the implantable devices with and without the CP Fiber wire.

There was a similar host tissue response to both the reinforced and non-reinforced compressive molded Reticulated Elastomeric Matrix 1 implantable devices. The healing response was characterized by an inflammatory reaction at the site of the host-graft interaction consisting of mainly mononuclear cell infiltration in week 1. Multinucleate giant cells increased in number throughout the course of the study. By week 2, an increasingly-organized connective tissue capsule surrounded the graft and connective tissue was beginning to fill the pores of the implantable device. The organization of the connective tissue progressively increased with time. The connective tissue was very mature within and surrounding the graft material by week 16. The amount of vasculature in the graft increased until week 8. No necrosis of the underlying muscle tissue was noted in any of the animals.

### Example 20

### Use of Reticulated Elastomeric Matrix 4 with a Selectively Non-Porous Surface in an Implantable Device with Multi-Filament Braided Fibers

Reticulated Elastomeric Matrix 4 is made by following the procedures described in Example 8. A square slab, measuring 50 mm in length and width and 2 mm in height, is cut from the matrix. Of the two surfaces of the slab with the greatest surface area, one is brought into contact with a heated plate (maintained at an elevated temperature in excess of 160° C.) in a nitrogen atmosphere to melt the contacted surface, thereby creating a relatively impervious layer, or a layer with low permeability relative to the reticulated elastomeric matrix, on one side of the slab. An implantable device, a square patch measuring 42 mm in length and width and 2 mm in height, is subsequently cut from the previously-described slab with the impervious layer. Multi-filament braided 4-0 polyester fibers (Telflex Medical; diameter equivalent to a 4-0 suture) are incorporated in the form of a grid into the square patch to form an implantable device that can be used as, e.g., a surgical mesh. The dimensions of the square grid are 8 mm×8 mm with 2 mm borders along each of the four sides.

### Example 21

### Use of Reticulated Elastomeric Matrix 4 with a Selectively Non-Porous Surface in an Implantable Device with Degradable Multi-Filament Braided Fibers

Reticulated Elastomeric Matrix 4 is made by following the procedures described in Example 8. A square slab, measuring 50 mm in length and width and 2 mm in height, is cut from the matrix. Of the two surfaces of the slab with the greatest surface area, one is coated with a solution of thermoplastic polycarbonate polyurethane dissolved in a mixture of 97% tetrahydrofuran and 3% dimethylformamide by volume. After the solvents evaporate, a thin coating is left on the pores of the contacted surface, thereby creating a relatively impervious layer, or a layer with low permeability relative to the reticulated elastomeric matrix, on one side of the slab. An implantable device, a square patch measuring 42 mm in length and width and 2 mm in height, is subsequently cut from the previously-described slab with the impervious layer. Degradable multi-filament braided fibers (Ethicon Inc.; copolymer of glycolide and lactide and diameter equivalent to a 4-0 VICRYL suture) are incorporated in the form of a grid into the square patch to form an implantable device that can be used, e.g., as a surgical mesh. The dimensions of the square grid are 8 mm×8 mm with 2 mm borders along each of the four sides.

### Example 22

### Use of Reticulated Elastomeric Matrix 4 with Braided Fiber Reinforcement in an Implantable Device for the Augmentation of the Sheep Rotator Cuff

An implantable device formed from Reticulated Elastomeric Matrix 4 and braided polyester fibers and in the shape of a rectangular patch measuring 40 mm in length, 20 mm in width, and 2 mm in thickness was made as described in Example 14 except that 7-0 braided polyester fibers were used. Such an implantable device was implanted in each Group 2 sheep as described below for healing of the rotator cuff tear and the infraspinatus tendon in the sheep chronic model to assess the implantable device's enhancement of the attachment of the infraspinatus tendon to the humerus.

A chronic defect was created in the right shoulder of each sheep. Skeletally mature, more than 3.5 year-old, Rambouillet X Columbia ewes (Ovis ares) weighing from about 60 Kg to about 100 Kg were used. 23 animals underwent this procedure. Under general anesthesia using aseptic conditions, a 6 cm skin incision was made over the right shoulder joint. The subcutaneous coli muscle was divided in line with the incision. The deltoid muscle was split along the tendinous division between its acromial and scapular heads. The superficial head and insertion of infraspinatus tendon was isolated. The infraspinatus was detached from the humerus and then wrapped with a 5 cm×3 cm sheet of PRECLUDE Dura Substitute (W.L. Gore and Associates, Flagstaff, Ariz.). The wound was closed using routine methods.

Four weeks later, the sheep were re-anesthetized and the sheet of PRECLUDE was removed. The former insertion site of the infraspinatus tendon was decorticated with a Hall orthopedic burr. A standard area of bone (1 cm×1 cm) was decorticated. In a control group with 11 animals (Group 1), after the placement of four Biosuture tack anchors (3.0 mm Biosuture tack anchors from Arthrex) in a 1 cm×1 cm square configuration in the humeral tuberosity, the infraspinatus tendon was grasped and reattached to the proximal humerus using two suture anchors and a Mason-Allen pattern stitch. Stated another way, in the control group the tendon was reattached to the bone without the implantable device.

In the other group with 12 animals (Group 2), an implantable device was placed on the top of the repair site so that there was about a 1 cm overhang on the tuberosity side. The remainder of the device extended onto the tendon. The anchor sutures used for the tendon attachment went through the implantable device with vertical mattress stitches, creating a layered construct consisting of implantable device and tendon. Laterally, the other two anchor sutures went through the device and tied the implantable device down to the tuberosity. All implantable device fixation stitches crossed at least on fiber element of the reinforcement grid in the device.

The Group 1 and 2 animals were euthanatized at 12 weeks after the second reattachment surgery. Nine shoulders from the group that received the implantable device (Group 2) and eight shoulders from the control group (Group 1) were collected and immediately prepared for biomechanical testing as follows. After removal of the extraneous soft tissue while leaving the humerus-infraspinatus tendon construct intact, several screws were drilled into both the proximal and distal humerus to further increased the purchase of the humerus in areas that were coupled to the metal fixtures using a polymethylmethacrylate (PMMA) potting material. Each test specimen was then mounted in a servo-hydraulic testing machine (Model 805 from MTS Corp., Eden Prairie, Minn.) using specially designed grips. The lower grip held the PMMA-potted end of the humerus. The upper grip was clamped onto the infraspinatus tendon with a brass cryo-grip, developed based on previous studies as a precaution to prevent slippage. The upper grip was moved at 0.5% strain/sec to provide a tensile load until specimen failure and the ultimate load (defined as the maximum load) reached by each specimen during the biomechanical test was recorded.

The average (from 8 animals) ultimate load for the control group (Group 1) was 762 Newtons with a standard deviation of 474 Newtons. The average (from 9 animals) ultimate load for the group that received the implantable device (Group 2) was 1,328 Newtons with a standard deviation of 427 Newtons. Using a standard one-way ANOVA statistical analysis and at a p-value of 0.05, the ultimate load for the group that received the implantable device (Group 2) was judged as significantly different from and higher than the control group (Group 1) that did not receive the device.

Histology analysis was done on three repaired shoulders from the control group (Group 1) that were not used in biomechanical testing and three repaired shoulders from the group that received the implantable device (Group 2) that were not used in biomechanical testing. Histologically, the implantable device material was found to be very inert. Very minimal inflammation response was evident. Tissue ingrowth was identified in all implantable devices with collagen fiber formation. The tissues also grew into the bone of the humerus.

### Example 23

### Synthesis and Properties of Reticulated Elastomeric Matrix 7

A reticulated cross-linked biodurable elastomeric polycarbonate urea-urethane matrix was made by the procedure described in Example 5 except that the ingredients used and their proportions are given in Table 14 below.

TABLE 14

| Ingredient | Parts by Weight |
|---|---|
| Polyol Component | 100 |
| Isocyanate Component | 53.55 |
| Isocyanate Index | 1.00 |
| Viscosity Modifier | 5.80 |
| Cell Opener | 2.00 |
| Distilled Water | 1.80 |
| B-8305 Surfactant | 1.20 |
| BF 2370 Surfactant | 1.20 |
| 33LV Catalyst | 0.35 |
| A-133 Catalyst | 0.15 |
| Glycerine | 1.15 |
| 1,4-Butanediol | 3.00 |

The average cell diameter or other largest transverse dimension of Reticulated Elastomeric Matrix 7, as determined from optical microscopy observations, was about 481 μm. SEM images of Reticulated Elastomeric Matrix 7 demonstrated, e.g., the network of cells interconnected via the open pores therein.

The following tests were carried out on the thus-formed Reticulated Elastomeric Matrix 7, obtained from reticulating the foam, using test methods based on ASTM Standard D3574. The density of Reticulated Elastomeric Matrix 7 was determined as described in Example 5; a density value of 4.96 lbs/ft³ (0.080 g/cc) was obtained.

Tensile tests were conducted on Reticulated Elastomeric Matrix 7 specimens as described in Example 5. The average post-reticulation tensile strength perpendicular to the foam-rise direction was determined to be about 50.2 psi (35,300 kg/m²). The post-reticulation elongation to break perpendicular to the foam-rise direction was determined to be about 162%. The average post-reticulation tensile strength parallel to the foam-rise direction was determined to be about 68.2 psi (48,000 kg/m²). The post-reticulation elongation to break parallel to the foam-rise direction was determined to be about 166%.

Compressive tests were conducted using Reticulated Elastomeric Matrix 7 specimens as described in Example 5. The post-reticulation compressive strength at 50% compression, parallel to the foam-rise direction, was determined to be about 3.31 psi (2,325 kg/m²).

The resilient recovery of Reticulated Elastomeric Matrix 7 was measured as described in Example 5. The results obtained are shown in Table 15.

TABLE 15

| | Test Specimen | | |
|---|---|---|---|
| No. of Cycles at | Orientation | | |
| 50% Compression ± 5% | Relative to Foam- | t-67% | t-90% |
| Strain at 1 Hz | Rise Direction | (sec) | (sec) |
| 100,000 (in air) | Parallel | — | 1630 |
| 100,000 (in water) | Parallel | — | 1140 |

Fluid permeability through Reticulated Elastomeric Matrix 7 was measured in the foam-rise direction as described in Example 5 using the Automated Liquid Permeameter, Model LP-101-A. The permeability of Reticulated Elastomeric Matrix 7 was determined to be 282 Darcy in the foam-rise direction.

Permeability was also measured after Reticulated Elastomeric Matrix 7 was compressed (perpendicular to the foam-rise direction) so as to reduce the available flow area, as described in Example 5. Line 1 in FIG. 11 is a plot of the Darcy permeability vs. available flow area for Reticulated Elastomeric Matrix 7. In FIG. 11, the 100% Available Flow Area represents uncompressed Reticulated Elastomeric Matrix 7 and demonstrates the highest permeability in the foam-rise direction, 282 Darcy. The permeability in the foam-rise direction for Reticulated Elastomeric Matrix 7 decreased to 136 Darcy when the available flow area after compression was reduced to 47.2% of the original area and to 95 Darcy when the available flow area after compression was reduced to 37.0% of the original area.

### Example 24

### Synthesis and Properties of Reticulated Elastomeric Matrix 8

A reticulated cross-linked biodurable elastomeric polycarbonate urea-urethane matrix was made by the procedure described in Example 7 except that the ingredients used and their proportions are given in Table 16 below. In partcular, a the surfactants B-8300 and B-5055 (each from Goldschmidt) were used in place of B-8305 surfactant for cell stabilization.

TABLE 16

| Ingredient | Parts by Weight |
|---|---|
| Polyol Component | 100 |
| Isocyanate Component | 49.18 |
| Isocyanate Index | 1.00 |
| Viscosity Modifier | 5.80 |
| Cell Opener | 2.00 |
| Distilled Water | 1.45 |
| B-8300 Surfactant | 0.45 |
| B-5055 Surfactant | 0.45 |
| BF 2370 Surfactant | 0.90 |
| 33LV Catalyst | 0.30 |
| A-133 Catalyst | 0.15 |
| Glycerine | 2.00 |
| 1,4-Butanediol | 2.00 |

The average cell diameter or other largest transverse dimension of Reticulated Elastomeric Matrix 8, as determined from optical microscopy observations, was about 512 μm. SEM images of Reticulated Elastomeric Matrix 8 demonstrated, e.g., the network of cells interconnected via the open pores therein.

The following tests were carried out on the thus-formed Reticulated Elastomeric Matrix 8, obtained from reticulating the foam, using test methods based on ASTM Standard D3574. The density of Reticulated Elastomeric Matrix 8 was determined as described in Example 5; a density value of 5.25 lbs/ft³ (0.084 g/cc) was obtained.

Blocks of Reticulated Elastomeric Matrix 8 were then annealed, unconstrained, in an oven at 110° C. for either 5 hours or 10 hours.

Tensile and compressive tests were conducted on unannealed and annealed Reticulated Elastomeric Matrix 8 specimens both perpendicular to and parallel to the foam-rise direction as described in Example 5. Additionally, the tensile modulus and compressive modulus, i.e., the initial slope of each corresponding stress vs. strain curve, were each calculated by determining the ratio of stress to strain at low strains. As demonstrated by the results shown below in Table 17, post-reticulation annealing at 110° C. for both 5 hours and 10 hours resulted in significantly increased mechanical performance of Reticulated Elastomeric Matrix 8. It should be noted that the density of Reticulated Elastomeric Matrix 8 remained substantially unchanged after annealing.

TABLE 17

| | | | After | | After | |
|---|---|---|---|---|---|---|
| | Post- | | Annealing | | Annealing | |
| | Reticulation, | | at 110° C. | | at 110° C. | |
| Property | No Annealing | | for 5 hours | | for 10 hours | |
| Tensile Strength, | 49.0 | psi | 61.7 | psi | 66.0 | psi |
| Perpendicular to | | | | | | |
| Foam-Rise Direction | | | | | | |
| Tensile Modulus, | 30.3 | psi | 34.7 | psi | 40.2 | psi |
| Perpendicular to | | | | | | |
| Foam-Rise Direction | | | | | | |
| Tensile Strength, | 64.9 | psi | 78.1 | | 82.2 | |
| Parallel to Foam-Rise | | | | | | |
| Direction | | | | | | |
| Tensile Modulus, | 46.8 | | 46.1 | | 60.2 | psi |
| Parallel to Foam-Rise | | | | | | |
| Direction | | | | | | |
| Compressive Strength | 2.1 | psi | 3.8 | psi | 4.4 | psi |
| at 50% Compression, | | | | | | |
| Parallel to Foam-Rise | | | | | | |
| Direction | | | | | | |
| Compressive Modulus, | 30.7 | psi | 56.2 | psi | 61.4 | psi |
| Parallel to Foam-Rise | | | | | | |
| Direction | | | | | | |

Disclosures Incorporated

The entire disclosure of each and every U.S. patent and patent application, each foreign and international patent publication and each other publication, and each unpublished patent application that is referenced in this specification, or elsewhere in this patent application, is hereby specifically incorporated herein, in its entirety, by the respective specific reference that has been made thereto.

While illustrative embodiments of the invention have been described above, it is understood that many and various modifications will be apparent to those in the relevant art, or may become apparent as the art develops. Such modifications are contemplated as being within the spirit and scope of the invention or inventions disclosed in this specification.

1-101. (canceled)

102. An implantable device comprising:
a reticulated elastomeric matrix comprising a biodurable polymeric material; and
a reinforcement in at least one dimension,
wherein said implantable device is suitably dimensioned for implantation into an anatomical site.

103. The implantable device of claim 102, further comprising at least one additional reticulated elastomeric matrix.

104. The implantable device of claim 102, wherein said reinforcement is a 2-dimensional reinforcement.

105. The implantable device of claim 102, wherein said reinforcement comprises at least one selected from the group consisting of fibers, meshes, wires, sutures, yarns, grids and any combinations thereof.

106. The implantable device of claim 105, wherein said reinforcement comprises a 2-dimensional mesh.

107. The implantable device of claim 105, wherein said reinforcement comprises mono-filament fiber, multi-filament yarn, braided multi-filament yarns, comingled mono-filament fibers, comingled multi-filament yarns, bundled mono-filament fibers, bundled multi-filament yarns, or any mixtures thereof.

108. The implantable device of claim 105, wherein said reinforcement comprises an amorphous polymer, a semi-crystalline polymer, a cross-linked polymer, a bioabsorbable polymer, a biodegradable polymer, an absorbable polymer, a non-biodegradable polymer, a non-bioabsorbable polymer, carbon, glass, ceramic or any mixtures thereof.

109. The implantable device of claim 102, wherein said reinforcement comprises a non-absorbable or non-biodegradable polymer.

110. The implantable device of claim 109, wherein said polymer is selected from the group consisting of polyesters, polyolefins, polyurethanes, polyamides, polyimides, polycarbonates, and any mixtures thereof.

111. The implantable device of claim 110, wherein said polymer is a polypropylene.

112. The implantable device of claim 102, wherein said reinforcement comprises a biodegradable, bioabsorbable or absorbable polymer.

113. The implantable device of claim 112, wherein said polymer is selected from the group consisting of:
(i) homopolymers and copolymers of lactic acid, glycolic acid, lactide, d-, l-, and meso lactide, glycolide, para-dioxanone, trimethylene carbonate, ∈-caprolactone and blends thereof;
(ii) polyanhydrides;
(iii) polyorthoesters;
(iv) polyoxaesters; and
(v) any mixture thereof.

114. The implantable device of claim 102, wherein said reinforcement is coated with a polymer.

115. The implantable device of claim 102, wherein at least a portion of an outer surface of said device is coated with a polymer coating.

116. The implantable device of claim 115, wherein said polymer coating comprises a biodegradable or absorbable polymer.

117. The implantable device of claim 115, wherein said polymer coating reduces the percentage of pores open at an exterior macro surface of said matrix.

118. The implantable device of claim 117, wherein said polymer coating substantially closes-off the pores open at an exterior macro surface of said matrix.

119. The implantable device of claim 116, wherein said biodegradable or absorbable polymer is selected from the group consisting of:
(i) homopolymers and copolymers of lactic acid, glycolic acid, lactide, d-, l-, and meso lactide, glycolide, para-dioxanone, trimethylene carbonate, ∈-caprolactone or a mixture thereof;
(ii) polyanhydrides;
(iii) polyorthoesters;
(iv) polyoxaesters; and
(v) any mixture thereof.

120. The implantable device of claim 115, wherein said polymer coating comprises a non-biodegradable or non-absorbable polymer.

121. The implantable device of claim 115, wherein the reticulated elastomeric matrix is coated with a film-forming biocompatible polymer in a liquid coating solution or in a melt state suitable to allow the formation of a biocompatible polymer film.

122. The implantable device of claim 102, wherein said reticulated elastomeric matrix is configured to permit cellular ingrowth and proliferation into the reinforced reticulated elastomeric matrix.

123. The implantable device of claim 102, wherein said matrix is formed from a material selected from the group consisting of polycarbonate polyurethane, polycarbonate polyurea urethane, polysiloxane polyurethane, polysiloxane polyurethane urea, polycarbonate polysiloxane polyurethane, polycarbonate polysiloxane polyurethane urea, polycarbonate hydrocarbon polyurethane, polycarbonate hydrocarbon polyurethane urea, or any mixture thereof.

124. The implantable device of claim 123, wherein said elastomeric matrix is formed by the reaction of diphenylmethane diisocyanate with a polyol,
wherein the diphenylmethane diisocyanate comprises 2,4′-diphenylmethane diisocyanate, 4,4′-diphenylmethane diisocyanate or any mixture thereof.

125. The implantable device of claim 102, wherein the implantable device is compressive molded.

126. The implantable device of claim 102, wherein the implantable device is annealed.
